(19) United States
(12) Patent Application Publication
(10) Pub. No.: US 2008/0306055 A1
(43) Pub. Date: Dec. 11, 2008

(54) Heterocyclic Mchr1 Antagonists And Their Use In Therapy

(75) Inventors: Bryan Egner, Molndal (SE); Fabrizio Giordanetto, Molndal (SE); Tord Inghardt, Molndal (SE)

Correspondence Address: Pepper Hamilton LLP, 400 Berwyn Park, 899 Cassatt Road, Berwyn, PA, 19312-1183, US

(73) Assignee: ASTRAZENECA AB, Södertälje (SE)

(21) Appl. No.: 11/721,767

(22) PCT Filed: Dec. 19, 2005
(86) PCT No.: PCT/SE2005/001966

§ 371 Date: Jun. 14, 2007

(30) Foreign Application Priority Data
Dec. 21, 2004 (SE) 0403119-1
Jul. 15, 2005 (SE) 0501686-0

(51) Int. Cl. A61K 31/541; A61K 31/454; C07D 401/02; A61K 31/4545; A61P 25/00; A61P 3/10; A61P 25/18; C07D 401/14; C07D 417/14

(52) U.S. Cl. 514/227.8; 546/208; 514/326; 514/318; 546/194; 544/60

(57) ABSTRACT

Compounds of formula I depicted below, pharmaceutical compositions containing them, processes for preparing the compounds, and their use in the treatment of obesity, type II diabetes, metabolic syndrome, psychiatric disorders, cognitive disorders, memory disorders, schizophrenia, epilepsy and related conditions, neurological disorders such as dementia, multiple sclerosis, Parkinson's disease, Huntington's chorea and Alzheimer's disease, and pain related disorders. The compounds are melanin concentrating hormone receptor 1 (MCHr1) antagonists.

### FIELD OF INVENTION

The present invention relates to certain compounds of formula I, to processes for preparing such compounds, to their use in the treatment of obesity, psychiatric and neurological disorders, and to pharmaceutical compositions containing them.

### BACKGROUND OF THE INVENTION

Melanin concentrating hormone (MCH) is a cyclic peptide that was first isolated from fish over 15 years ago. In mammals, MCH gene expression is localised to the ventral aspect of the zona inserta and the lateral hypothalamic area (Breton et al., Molecular and Cellular Neurosciences, vol. 4, 271-284 (1993)). The latter region of the brain is associated with the control of behaviours such as eating and drinking, with arousal and with motor activity (Baker, B., Trends Endocrinol. Metab. 5: 120-126 (1994), vol. 5, No. 3, 120-126 (1994)). Although the biological activity in mammals has not been fully defined, recent work has indicated that MCH promotes eating and weight gain (U.S. Pat. No. 5,849,708). Thus, MCH and its agonists have been proposed as treatments for anorexia nervosa and weight loss due to AIDS, renal disease, or chemotherapy. Similarly, antagonists of MCH can be used as a treatment for obesity and other disorders characterised by compulsive eating and excessive body weight. MCHr1 projections are found throughout the brain, including the spinal cord, an area important in processing nociception, indicates that agents acting through MCHr1, such as compounds of formula I, will be useful in treating pain.

Two receptors for MCH (MCH receptor 1 (MCHr1) (Shimomura et al. Biochem Biophys Res Commun 1999 Aug. 11; 261(3):622-6) & MCH receptor 2 (MCHr2) (Hilol et al. J Biol. Chem. 2001 Jun. 8; 276(23):20125-9)) have been identified in humans, while only one (MCHr1) is present in rodent species (Tan et al. Genomics 2002 June; 79(6):785-92). In mice lacking MCHr1, there is no increased feeding response to MCH, and a lean phenotype is seen, suggesting that this receptor is responsible for mediating the feeding effect of MCH (Marsh et al. Proc. Natl. Acad. Sci. USA, 2002 Mar. 5; 99(5):3240-5). In addition, MCHr1 antagonists have been demonstrated to block the feeding effects of MCH (Takekawa et al. Eur. J. Pharmacol. 2002 Mar. 8; 438(3):129-35), and to reduce body weight & adiposity in diet-induced obese rats (Borowsky et al. Nature Med. 2002 August; 8(8):825-30). The conservation of distribution and sequence of MCHr.1 suggest a similar role for this receptor in man and rodent species. Hence, MCHr1 antagonists have been proposed as a treatment for obesity and other disorders characterised by excessive eating and body weight.

WO 03/106452 discloses certain 1-substituted-4-(substituted amino)piperidines which are alleged to be MCHr1 antagonists.

An abstract (No. 343 Vu V. Ma et al.,) from the 224^{th} ACS meeting in Boston, Mass., USA presents a MCH receptor antagonist for the potential treatment of obesity, with the following structure:

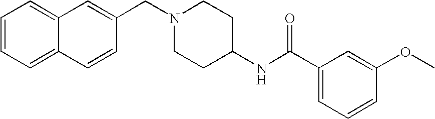

WO 01/14333 discloses that compounds of the following formula:

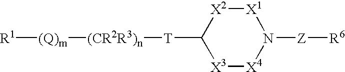

wherein

Z is CR⁴R⁵, C(O) or CR⁴R⁵-Z¹;

Z¹ is C₁₋₄ alkylene (such as CH₂), C₂₋₄ alkenylene (such as CH═CH) or C(O)NH;

R¹ represents a C₁-C₁₂ alkyl group optionally substituted by one or more substituents independently selected from cyano, hydroxyl, C₁-C₆ alkoxy (such as methoxy or ethoxy), C₁-C₆ alkylthio (such as methylthio), C₃₋₇ cycloalkyl (such as cyclopropyl), C₁-C₆ alkoxycarbonyl (such as methoxycarbonyl) and phenyl (itself optionally substituted by one or more of halogen, nitro, cyano, C₁-C₆ alkyl, C₁-C₆ haloalkyl (such as CF₃), phenyl(C₁-C₆ alkyl) (such as benzyl), C₁-C₆ alkoxy, C₁-C₆ haloalkoxy, S(O)₂(C₁-C₆ alkyl), C(O)NH₂, carboxy or C₁-C₆ alkoxycarbonyl); or

R¹ represents C₂-C₆ alkenyl optionally substituted by phenyl (itself optionally substituted by one or more of halogen, nitro, cyano, C₁-C₆ alkyl, C₁-C₆ haloalkyl, phenyl(C₁-C₆ alkyl), C₁-C₆ alkoxy, C₁-C₆ haloalkoxy, S(O)₂(C₁-C₆ alkyl), C(O)NH₂, carboxy or C₁-C₆ alkoxycarbonyl); or

R¹ represents a 3- to 14-membered saturated or unsaturated ring system which optionally comprises up to two ring carbon atoms that form carbonyl groups and which optionally further comprises up to 4 ring heteroatoms independently selected from nitrogen, oxygen and sulphur, wherein the ring system is optionally substituted by one or more substituents independently selected from: halogen, cyano, nitro, oxo, hydroxyl, C₁-C₈ alkyl, C₁-C₆ hydroxyalkyl, C₁-C₆ haloalkyl, C₁₋₆ alkoxy(C₁-C₆ alkyl), C₃-C₇ cycloalkyl(C₁-C₆ alkyl), C₁-C₆ alkylthio(C₁-C₆ alkyl), C₁-C₆ alkylcarbonyloxy(C₁-C₆ alkyl), C₁-C₆ alkylS(O)₂(C₁-C₆ alkyl), aryl(C₁-C₆ alkyl), heterocyclyl(C₁-C₆ alkyl), arylS(O)₂(C₁-C₆ alkyl), heterocyclylS(O)₂(C₁-C₆ alkyl), aryl(C₁-C₆ alkyl)S(O)₂, heterocyclyl(C₁-C₆ alkyl)S(O)₂, C₂-C₆ alkenyl, C₁-C₆ alkoxy, carboxy-substituted C₁-C₆ alkoxy, C₁-C₆ haloalkoxy, C₁-C₆ hydroxyalkoxy, C₁-C₆ alkylcarboxy-substituted C₁-C₆ alkoxy, aryloxy, heterocyclyloxy, C₁-C₆ alkylthio, C₃-C₇ cycloalkyl(C₁-C₆ alkylthio), C₃-C₆ alkynylthio, C₁-C₆ alkylcarbonylamino, C₁-C₆ haloalkylcarbonylamino, SO₃H, —NR⁷R⁸, —C(O)NR²³R²⁴, S(O)₂NR¹⁸R¹⁹, S(O)₂R²⁰, R²⁵C(O), carboxyl, C₁-C₆ alkoxycarbonyl, aryl and heterocyclyl; wherein the foregoing aryl and heterocyclyl moieties are optionally substituted by one or more of halogen, oxo, hydroxy, nitro, cyano, C₁-C₆ alkyl, C₁-C₆ haloalkyl, phenyl(C₁-C₆ alkyl), C₁-C₆ alkoxy, C₁-C₆ haloalkoxy, S(O)₂(C₁-C₆ alkyl), C(O)NH₂, carboxy or C₁-C₆ alkoxycarbonyl;

m is 0 or 1;

Q represents an oxygen or sulphur atom or a group NR⁹, C(O), C(O)NR⁹, NR⁹C(O) or CH═CH;

n is 0, 1, 2, 3, 4, 5 or 6 provided that when n is 0, then m is 0;

each R² and R³ independently represents a hydrogen atom or a C₁-C₄ alkyl group, or (CR²R³) represents C₃-C₇ cycloalkyl optionally substituted by C₁-C₄ alkyl;

T represents a group NR¹⁰, C(O)NR¹⁰, NR¹¹C(O)NR¹⁰ or C(O)NR¹⁰NR¹¹;

X¹, X², X³ and X⁴ are, independently, CH₂, CHR¹² {wherein each R¹² is, independently, C₁-C₄ alkyl or C₃-C₇ cycloalkyl(C₁-C₄ alkyl)} or C═O; or, when they are CHR²¹, the R¹² groups of X¹ and X³ or X⁴, or, X² and X³ or X⁴ join to form a two or three atom chain which is CH₂CH₂, CH₂CH₂CH₂, CH₂OCH₂ or CH₂SCH₂; provided always that at least two of X¹, X², X³ and X⁴ are CH₂;

R⁴ and R⁵ each independently represent a hydrogen atom or a C₁-C₄ alkyl group;

R⁶ is aryl or heterocyclyl, both optionally substituted by one or more of: halogen, cyano, nitro, oxo, hydroxyl, C₁-C₈ alkyl, C₁-C₆ hydroxyalkyl, C₁-C₆ haloalkyl, C₁₋₆ alkoxy(C₁-C₆ alkyl), C₃-C₇ cycloalkyl(C₁-C₆ alkyl), C₁-C₆ alkylthio(C₁-C₆ alkyl), C₁-C₆ alkylcarbonyloxy(C₁-C₆ alkyl), C₁-C₆ alkylS(O)₂(C₁-C₆ alkyl), aryl(C₁-C₆ alkyl), heterocyclyl(C₁-C₆ alkyl), arylS(O)₂(C₁-C₆ alkyl), heterocyclylS(O)₂(C₁-C₆ alkyl), aryl(C₁-C₆ alkyl)S(O)₂, heterocyclyl(C₁-C₆ alkyl)S(O)₂, C₂-C₆ alkenyl, C₁-C₆ alkoxy, carboxy-substituted C₁-C₆ alkoxy, C₁-C₆ haloalkoxy, C₁-C₆ hydroxyalkoxy, C₁-C₆ alkylcarboxy-substituted C₁-C₆ alkoxy, aryloxy, heterocyclyloxy, C₁-C₆ alkylthio, C₃-C₇ cycloalkyl(C₁-C₆ alkylthio), C₃-C₆ alkynylthio, C₁-C₆ alkylcarbonylamino, C₁-C₆ haloalkylcarbonylamino, SO₃H, —NR¹⁶R¹⁷, C(O)NR²R²², S(O)₂NR¹³R¹⁴, S(O)₂R¹⁵, R²⁶C(O), carboxyl, C₁-C₆ alkoxycarbonyl, aryl and heterocyclyl; wherein the foregoing aryl and heterocyclyl moieties are optionally substituted by one or more of halogen, nitro, cyano, C₁-C₆ alkyl, C₁-C₆ haloalkyl, phenyl(C₁-C₆ alkyl), C₁-C₆ alkoxy, C₁-C₆ haloalkoxy, S(O)₂(C₁-C₆ alkyl), C(O)NH₂, carboxy or C₁-C₆ alkoxycarbonyl;

R⁷, R⁸, R⁹, R¹⁰, R¹¹, R¹³, R¹⁴R¹⁶, R¹⁷, R¹⁸, R¹⁹, R²¹, R²², R²³ and R²⁴ are, independently hydrogen, C₁-C₆ alkyl, C₁-C₆ haloalkyl, C₁-C₆ hydroxyalkyl, C₃-C₇ cycloalkyl, C₃-C₇ cycloalkyl(C₁-C₄ alkyl) or phenyl(C₁-C₆ alkyl); and,

R¹⁵ and R²⁰ are, independently, C₁-C₆ alkyl, C₁-C₆ hydroxyalkyl, C₃-C₆ cycloalkyl, C₃-C₇ cycloalkyl(C₁-C₄ alkyl) or C₁-C₆ alkyl optionally substituted by phenyl;

R²⁵ and R²⁶ are, independently, C₁-C₆ alkyl or phenyl (optionally substituted by one or more of halogen, nitro, cyano, C₁-C₆ alkyl, C₁-C₆ haloalkyl, phenyl(C₁-C₆ alkyl), C₁-C₆ alkoxy, C₁-C₆ haloalkoxy, S(O)₂(C₁-C₆ alkyl), C(O)NH₂, carboxy or C₁-C₆ alkoxycarbonyl);

or a pharmaceutically acceptable salt thereof, or solvate thereof, or a solvate of a salt thereof;

provided that when T is C(O)NR¹⁰ and R¹ is optionally substituted phenyl then n is not 0, have activity as modulators of chemokine receptor activity.

There is an unmet need for MCHr1 antagonists that are more potent, more selective, more bioavailable and produce less side effects than known compounds in this field.

### SUMMARY OF THE INVENTION

It is an object of the present invention to provide compounds, which are useful in treating obesity and related disorders, psychiatric disorders, neurological disorders and pain. This object has been reached in that a compound of formula I has been provided for use as a MCHr1 antagonist.

According to another aspect of the invention a pharmaceutical formulation is provided comprising a compound of formula I, and a pharmaceutically acceptable adjuvant, diluent or carrier.

According to a further aspect of the invention, the use of a compound of formula I is provided, in the preparation of a medicament for the treatment or prophylaxis of conditions associated with obesity.

According to yet another aspect of the invention, a method is provided of treating obesity, psychiatric disorders, anxiety, anxio-depressive disorders, depression, bipolar disorder, ADHD, cognitive disorders, memory disorders, schizophrenia, epilepsy, and related conditions, and neurological disorders and pain related disorders, comprising administering a pharmacologically effective amount of a compound of Formula I to a patient in need thereof.

According to another aspect of the invention, a process for the preparation of compounds of formula I is provided.

According to a further aspect of the invention, a method is provided of treating obesity, type II diabetes, Metabolic syndrome and prevention of type II diabetes comprising administering a pharmacologically effective amount of a compound of formula I to a patient in need thereof.

### DESCRIPTION OF THE INVENTION

The invention relates to compounds of the general formula (I)

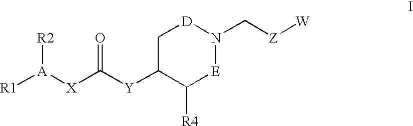

A represents N, a C₁₋₄ alkyl group, a C₂₋₄ alkenyl group, C₃₋₈ cycloalkyl, adamantyl, azetidinyl, pyrrolidinyl, piperidinyl, piperazinyl, morpholinyl, 1,3 oxazidinyl, tetrahydro-pyridinyl, or spiro[indene-1,4′-piperidinyl];

wherein said C₁₋₄ alkyl group or C₂₋₄ alkenyl group is optionally substituted by one or more fluoro;

X represents a bond or NR³,

wherein A and X do not both represent nitrogen;
wherein when A is azetidinyl, 1,3 oxazidinyl, pyrrolidinyl, piperidinyl, piperazinyl, morpholinyl, tetrahydropyridinyl, or spiro[indene-1,4′-piperidinyl]; the nitrogen atom in A is directly attached to C(O),

R¹ and R² independently represent H, C₁₋₆ alkyl, a C₂₋₆ alkenyl group, C₃₋₁₀ cycloalkyl, CONR^{a}R^{b} in which R^{a} and R^{b} independently represent H, a C₁₋₄ alkyl group or R^{a} and R^{b}, together with the nitrogen to which they are attached, form a 4 to 8 membered heterocyclic ring;

phenyl or naphthyl; or
a heterocyclic group selected from pyrrolyl, imidazolyl, furyl, thienyl, thiazolyl, isothiazolyl, thiadiazolyl, pyrazolyl, oxazolyl, isoxazolyl, pyridyl, pyrazinyl, pyrimidinyl, pyridazinyl, quinolinyl, isoquinolyl, quinazolyl, indolyl, benzofuranyl, benzo[b]thienyl, benzimidazolyl, benzothiazolyl, 1,4-benzodioxinyl, 1,3-benzodioxolyl, piperidinyl, morpholinyl, 1,4-oxazepanyl, or 4,4-dioxothiomorpholinyl;
wherein R¹ or R² are optionally substituted by one or more of the following:
cyano
halo
hydroxy
oxo
a C₁₋₄ alkyl group optionally substituted by one or more fluoro;
a C₁₋₄ alkoxy group optionally substituted by one or more fluoro;
a group NCOR^{a}R^{b} or CONR^{a}R^{b} in which R^{a} and R^{b} independently represent a C₁₋₃ alkyl group;
a group SO₂C₁₋₄alkyl, optionally substituted by one or more fluoro;
an aryl or heteroaryl group selected from thiadiazolyl, pyrazolyl, phenyl, phenoxy, 2-pyridyl or 3-pyridyl wherein said aryl or heteroaryl group may optionally be further substituted by one or more of the following:
   cyano,
   halo,
   hydroxy,
   a C₁₋₄ alkyl group optionally substituted by one or more fluoro;
   a C₁₋₄ alkoxy group optionally substituted by one or more fluoro;
   a group NCOR^{a}R^{b} or CONR^{a}R^{b} in which R^{a} and R^{b} independently represent a
   C₁₋₃ alkyl group;
   a group SO₂C₁₋₄alkyl, optionally substituted by one or more fluoro;

R¹ and/or R² is optionally linked to A via oxygen or via a C₁₋₄ alkyl group, wherein one of the carbon atoms in said C₁₋₄ alkyl group optionally is replaced with an oxygen atom,

Y represents NR³, C(R⁵, R⁶) or a bond,

wherein at least one of A, X or Y is N, NR³ or a nitrogen-containing heterocyclic ring,

R³, R⁵ and R⁶ independently represent H or a C₁₋₄ alkyl group,

D represents (CH₂)ₙ, wherein n is 0 or 1 and E represents (CH₂)ₘ, wherein m is 0 or 1,

R⁴ represents H or, when m and n are both 1, R₄ represents H or F,

Z represents 2,5-thienyl, 2,5-furyl, or pyrrolyl, optionally substituted by one or more of the following: cyano, halo, a C₁₋₄ alkyl group optionally substituted by one or more fluoro, a C₁₋₄ alkoxy group optionally substituted by one or more fluoro,

W represents phenyl, 2-pyridyl or 3-pyridyl each of which is optionally substituted by one or more of the following: cyano, halo, a C₁₋₄ alkyl group optionally substituted by one or more fluoro, a C₁₋₄ alkoxy group optionally substituted by one or more fluoro, a trifluoromethylsulfonyl or a 2,2′-difluoro-oxolanyl group (fused with two adjacent aromatic carbon atoms in W),

as well as tautomers, optical isomers and racemates thereof as well as pharmaceutically acceptable salts thereof,
with the proviso that when Y represents NR³ then A-X does not represent OCH₂, CH₂CH₂ or CH—CH, wherein each of the carbon atom may optionally be substituted by 1 to 2 methyl groups and/or 1 to 2 fluoro.

The invention further relates to compounds of the general formula (Ia)

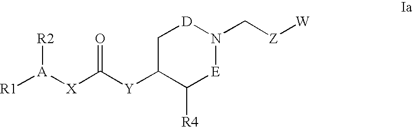

A represents N, a C₁₋₄ alkyl group, a C₂₋₄ alkenyl group, C₃₋₈ cycloalkyl, adamantyl, pyrrolidinyl, piperidinyl, piperazinyl, morpholinyl, tetrahydropyridinyl, or spiro[indene-1,4′-pipeiidinyl];

wherein said C₁₋₄ alkyl group or C₂₋₄ alkenyl group is optionally substituted by one or more fluoro;

X represents a bond or NR³,

wherein A and X do not both represent nitrogen;
wherein when A is pyrrolidinyl, piperidinyl, piperazinyl, morpholinyl, tetrahydropyridinyl, or spiro[indene-1,4′-piperidinyl]; the nitrogen atom in A is directly attached to C(O),

R¹ and R² independently represent H, C₁₋₆ alkyl, a C₂₋₆ alkenyl group, C₃₋₈ cycloalkyl, CONR^{a}R^{b} in which R^{a} and R^{b} independently represent H, a C₁₋₄ alkyl group or R^{a} and R^{b}, together with the nitrogen to which they are attached, form a 4 to 8 membered heterocyclic ring;

phenyl or naphthyl; or
a heterocyclic group selected from pyrrolyl, imidazolyl, furyl, thienyl, thiazolyl, isothiazolyl, thiadiazolyl, pyrazolyl, oxazolyl, isoxazolyl, pyridyl, pyrazinyl, pyrimidinyl, pyridazinyl, quinolinyl, isoquinolyl, quinazolyl, indolyl, benzofuranyl, benzo[b]thienyl, benzimidazolyl, benzothiazolyl, 1,4-benzodioxinyl or 1,3-benzodioxolyl;
wherein R¹ or R² are optionally substituted by one or more of the following:
cyano
halo
hydroxy
a C₁₋₄ alkyl group optionally substituted by one or more fluoro;
a C₁₋₄ alkoxy group optionally substituted by one or more fluoro;
a group NCOR^{a}R^{b} or CONR^{a}R^{b} in which R^{a} and R^{b} independently represent a C₁₋₃ alkyl group;
a group SO₂C₁₋₄alkyl, optionally substituted by one or more fluoro;
an aryl or heteroaryl group selected from thiadiazolyl, pyrazolyl, phenyl, phonoxy, 2-pyridyl or 3-pyridyl wherein said aryl or heteroaryl group may optionally be further substituted by one or more of the following:
   cyano,
   halo,
   hydroxy,
   a C₁₋₄ alkyl group optionally substituted by one or more fluoro;
   a C₁₋₄ alkoxy group optionally substituted by one or more fluoro;
   a group NCOR^{a}R^{b} or CONR^{a}R^{b} in which R^{a} and R^{b} independently represent a C₁₋₃ alkyl group;
   a group SO₂C₁₋₄alkyl, optionally substituted by one or more fluoro;

R¹ and/or R² is optionally linked to A via oxygen or via a C₁₋₄ alkyl group, wherein one of the carbon atoms in said C₁₋₄ alkyl group optionally is replaced with an oxygen atom,

Y represents NR³, C(R⁵, R⁶) or a bond,

wherein at least one of A, X or Y is N, NR³ or a nitrogen-containing heterocyclic ring,

R³, R⁵ and R⁶ independently represent H or a C₁₋₄ alkyl group,

D represents (CH₂)ₙ, wherein n is 0 or 1 and E represents (CH₂)ₘ, wherein m is 0 or 1,

R⁴ represents H or, when m and n are both 1, R₄ represents H or F,

Z represents 2,5-thienyl, 2,5-furyl, or pyrrolyl, optionally substituted by one or more of the following: cyano, halo, a C₁₋₄ alkyl group optionally substituted by one or more fluoro, a C₁₋₄ alkoxy group optionally substituted by one or more fluoro,

W represents phenyl, 2-pyridyl or 3-pyridyl each of which is optionally substituted by one or more of the following: cyano, halo, a C₁₋₄ alkyl group optionally substituted by one or more fluoro, a C₁₋₄ alkoxy group optionally substituted by one or more fluoro, a trifluoromethylsulfonyl or a 2,2′-difluoro-oxolanyl group (fused with two adjacent aromatic carbon atoms in W),

as well as tautomers, optical isomers and racemates thereof as well as pharmaceutically acceptable salts thereof,
with the proviso that when Y represents NR³ then A-X does not represent OCH₂, CH₂CH₂ or CH═CH, wherein each of the carbon atom may optionally be substituted by 1 to 2 methyl groups and/or 1 to 2 fluoro.

In group A, R¹ and R² is either attached to the same atom or to different atoms.

The invention further relates to compounds of formula Ib

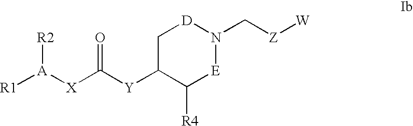

A represents azetidinyl, or 1,3 oxazidinyl,

X represents a bond,

wherein the nitrogen atom in A is directly attached to C(O),

R¹ and R² independently represent H, C₁₋₆ alkyl, a C₂₋₆ alkenyl group, C₃₋₁₀ cycloalkyl, CONR^{a}R^{b} in which R^{a} and R^{b} independently represent H, a C₁₋₄ alkyl group or R^{a} and R^{b}, together with the nitrogen to which they are attached, form a 4 to 8 membered heterocyclic ring;

phenyl or naphthyl; or
a heterocyclic group selected from pyrrolyl, imidazolyl, furyl, thienyl, thiazolyl, isothiazolyl, thiadiazolyl, pyrazolyl, oxazolyl, isoxazolyl, pyridyl, pyrazinyl, pyrimidinyl, pyridazinyl, quinolinyl, isoquinolyl, quinazolyl, indolyl, benzofuranyl, benzo[b]thienyl, benzimidazolyl, benzothiazolyl, 1,4-benzodioxinyl, 1,3-benzodioxolyl, piperidinyl, morpholinyl, 1,4-oxazepanyl, or 4,4-dioxothiomorpholinyl;
wherein R¹ or R² are optionally substituted by one or more of the following:
cyano
halo
hydroxy
a C₁₋₄ alkyl group optionally substituted by one or more fluoro;
a C₁₋₄ alkoxy group optionally substituted by one or more fluoro;
a group NCOR^{a}R^{b} or CONR^{a}R^{b} in which R^{a} and R^{b} independently represent a C₁₋₃ alkyl group;
a group SO₂C₁₋₄alkyl, optionally substituted by one or more fluoro;
an aryl or heteroaryl group selected from thiadiazolyl, pyrazeolyl, phenyl, pbenoxy, 2-pyridyl or 3-pyridyl wherein said aryl or heteroaryl group may optionally be further substituted by one or more of the following:
   cyano,
   halo,
   is hydroxy,
   a C₁₋₄ alkyl group optionally substituted by one or more fluoro;
   a C₁₋₄ alkoxy group optionally substituted by one or more fluoro;
   a group NCOR^{a}R^{b} or CONR^{a}R^{b} in which R^{a} and R^{b} independently represent a C₁₋₃ alkyl group;
   a group SO₂C₁₋₄alkyl, optionally substituted by one or more fluoro;

R¹ and/or R² is optionally linked to A via oxygen or via a C₁₋₄ alkyl group, wherein one of the carbon atoms in said C₁₋₄ alkyl group optionally is replaced with an oxygen atom,

Y represents NR³, C(R⁵, R⁶) or a bond,

wherein at least one of A, X or Y is N, NR³ or a nitrogen-containing heterocyclic ring,

R³, R⁵ and R⁶ independently represent H or a C₁₋₄ alkyl group,

D represents (CH₂), wherein n is 0 or 1 and E represents (CH₂)ₘ, wherein m is 0 or 1,

R⁴ represents H or, when m and n are both 1, R₄ represents H or F,

Z represents 2,5-thienyl, 2,5-furyl, or pyrrolyl, optionally substituted by one or more of the following: cyano, halo, a C₁₋₄ alkyl group optionally substituted by one or more fluoro, a C₁₋₄ alkoxy group optionally substituted by one or more fluoro,

W represents phenyl, 2-pyridyl or 3-pyridyl each of which is optionally substituted by one or more of the following: cyano, halo, a C₁₋₄ alkyl group optionally substituted by one or more fluoro, a C₁₋₄ alkoxy group optionally substituted by one or more fluoro, a trifluoromethylsulfonyl or a 2,2′-difluoro-oxolanyl group (fused with two adjacent aromatic carbon atoms in W),

as well as tautomers, optical isomers and racemates thereof as well as pharmaceutically acceptable salts thereof,
with the proviso that when Y represents NR³ then A-X does not represent OCH₂, CH₂CH₂ or CH═CH, wherein each of the carbon atom may optionally be substituted by 1 to 2 methyl groups and/or 1 to 2 fluoro.

The invention further relates to compounds of formula Ic

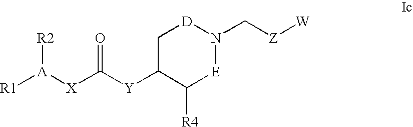

A represents N, a C₁₋₄ alkyl group, a C₂₋₄ alkenyl group, C₃₋₈ cycloalkyl, adamantyl, azetidinyl, pyrrolidinyl, piperidinyl, piperazinyl, morpholinyl, 1,3 oxazidinyl, tetrahydro-pyridinyl, or spiro[indene-1,4′-piperidinyl];

wherein said C₁₋₄ alkyl group or C₂₋₄ alkenyl group is optionally substituted by one or more fluoro;

X represents a bond or NR³,

wherein A and X do not both represent nitrogen;
wherein when A is azetidinyl, 1,3 oxazidinyl, pyrrolidinyl, piperidinyl, piperazinyl, morpholinyl, tetrahydropyridinyl, or spiro[indene-1,4′-piperidinyl]; the nitrogen atom in A is directly attached to C(O),

R¹ and R² independently represent C₉₋₁₀ cycloalkyl; or

a heterocyclic group selected from piperidinyl, morpholinyl, 1,4-oxazepanyl, or 4,4-dioxothiomorpholinyl;
wherein R¹ or R² are optionally substituted by one or more of the following:
cyano
halo
hydroxy
a C₁₋₄ alkyl group optionally substituted by one or more fluoro;
a C₁₋₄ alkoxy group optionally substituted by one or more fluoro;
a group NCOR^{a}R^{b} or CONR^{a}R^{b} in which R^{a} and R^{b} independently represent a C₁₋₃ alkyl group;
a group SO₂C₁₋₄alkyl, optionally substituted by one or more fluoro;
an aryl or heteroaryl group selected from thiadiazolyl, pyrazolyl, phenyl, phenoxy, 2-pyridyl or 3-pyridyl wherein said aryl or heteroaryl group may optionally be further substituted by one or more of the following:
   cyano,
   halo,
   hydroxy,
   a C₁₋₄ alkyl group optionally substituted by one or more fluoro;
   a C₁₋₄ alkoxy group optionally substituted by one or more fluoro;
   a group NCOR^{a}R^{b} or CONR^{a}R^{b} in which R^{a} and R^{b} independently represent a C₁₋₃ alkyl group;
   a group SO₂C₁₋₄alkyl, optionally substituted by one or more fluoro;

R¹ and/or R² is optionally linked to A via oxygen or via a C₁₋₄ alkyl group, wherein one of the carbon atoms in said C₁₋₄ alkyl group optionally is replaced with an oxygen atom,

Y represents NR³, C(R⁵, R⁶) or a bond,

wherein at least one of A, X or Y is N, NR³ or a nitrogen-containing heterocyclic ring,

R³, R⁵ and R⁶ independently represent H or a C₁₋₄ alkyl group,

D represents (CH₂)ₙ, wherein n is 0 or 1 and E represents (CH₂)ₘ, wherein m is 0 or 1,

R⁴ represents H or, when m and n are both 1, R₄ represents H or F,

Z represents 2,5-thienyl, 2,5-furyl, or pyrrolyl, optionally substituted by one or more of the following: cyano, halo, a C₁₋₄ alkyl group optionally substituted by one or more fluoro, a C₁₋₄ alkoxy group optionally substituted by one or more fluoro,

W represents phenyl, 2-pyridyl or 3-pyridyl each of which is optionally substituted by one or more of the following: cyano, halo, a C₁₋₄ alkyl group optionally substituted by one or more fluoro, a C₁₋₄ alkoxy group optionally substituted by one or more fluoro, a trifluoromethylsulfonyl or a 2,2′-difluoro-oxolanyl group (fused with two adjacent aromatic carbon atoms in W),

as well as tautomers, optical isomers and racemates thereof as well as pharmaceutically acceptable salts thereof,
with the proviso that when Y represents NR³ then A-X does not represent OCH₂, CH₂CH₂ or CH═CH, wherein each of the carbon atom may optionally be substituted by 1 to 2 methyl groups and/or 1 to 2 fluoro.
The invention further relates to compounds of the general formula (Id)

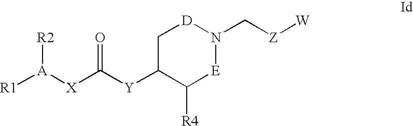

to A represents N, a C₁₋₄ alkyl group, a C₂₋₄ alkenyl group, C₃₋₈ cycloalkyl, adamantyl, azetidinyl, pyrrolidinyl, piperidinyl, piperazinyl, morpholinyl, 1,3 oxazidinyl, tetrahydro-pyridinyl, or spiro[indene-1,4′-piperidinyl];

wherein said C₁₋₄ alkyl group or C₂₋₄ alkenyl group is optionally substituted by one or more fluoro;

X represents a bond or NR³,

wherein A and X do not both represent nitrogen;
wherein when A is azetidinyl, 1,3 oxazidinyl, pyrrolidinyl, piperidinyl, piperazinyl, morpholinyl, tetrahydropyridinyl, or spiro[indene-1,4′-piperidinyl]; the nitrogen atom in A is directly attached to C(O),

R¹ and R² independently represent H, C₁₋₆ alkyl, a C₂₋₆ alkenyl group, C₃₋₁₀ cycloalkyl, CONR^{a}R^{b} in which R^{a} and R^{b} independently represent H, a C₁₋₄ alkyl group or R^{a} and R^{b}, together with the nitrogen to which they are attached, form a 4 to 8 membered heterocyclic ring;

phenyl or naphthyl; or
a heterocyclic group selected from pyrrolyl, imidazolyl, furyl, thienyl, thiazolyl, isothiazolyl, thiadiazolyl, pyrazolyl, oxazolyl, isoxazolyl, pyridyl, pyrazinyl, pyrimidinyl, pyridazinyl, quinolinyl, isoquinolyl, quinazolyl, indolyl, benzofuranyl, benzo[b]thienyl, benzimidazolyl, benzothiazolyl, 1,4-benzodioxinyl, 1,3-benzodioxolyl, piperidinyl, morpholinyl, 1,4-oxazepanyl, or 4,4-dioxothiomorpholinyl;
wherein R¹ and/or R² are substituted by oxo,

R¹ and/or R² is optionally linked to A via oxygen or via a C₁₋₄ alkyl group, wherein one of the carbon atoms in said C₁₋₄ alkyl group optionally is replaced with an oxygen atom,

Y represents NR³, C(R⁵, R⁶) or a bond,

wherein at least one of A, X or Y is N, NR³ or a nitrogen-containing heterocyclic ring,

R³, R⁵ and R⁶ independently represent H or a C₁₋₄ alkyl group,

D represents (CH₂)ₙ, wherein n is 0 or 1 and E represents (CH₂)ₘ, wherein m is 0 or 1,

R⁴ represents H or, when m and n are both 1, R₄ represents H or F,

Z represents 2,5-thienyl, 2,5-furyl, or pyrrolyl, optionally substituted by one or more of the following: cyano, halo, a C₁₋₄ alkyl group optionally substituted by one or more fluoro, a C₁₋₄ alkoxy group optionally substituted by one or more fluoro,

W represents phenyl, 2-pyridyl or 3-pyridyl each of which is optionally substituted by one or more of the following: cyano, halo, a C₁₋₄ alkyl group optionally substituted by one or more fluoro, a C₁₋₄ alkoxy group optionally substituted by one or more fluoro, a trifluoromethylsulfonyl or a 2,2′-difluoro-oxolanyl group (fused with two adjacent aromatic carbon atoms in W),

as well as tautomers, optical isomers and racemates thereof as well as pharmaceutically acceptable salts thereof,
with the proviso that when Y represents NR³ then A-X does not represent OCH₂, CH₂CH₂ or CH═CH, wherein each of the carbon atom may optionally be substituted by 1 to 2 methyl groups and/or 1 to 2 fluoro.

Particular groups now follow in which some of X, Y, Z, W, and R¹ in compounds of formula I-Ib are further defined. It will be understood that such group definitions may be used where appropriate with any of the other group definitions, claims or embodiments defined hereinbefore or hereinafter.

In another group of compounds of formula I-Ib, all compounds covered by claim 1 in WO 01/14333 are excluded.

In a particular group of compounds of formula I, Z represents 1, 3-1H pyrrolyl (in which the heteroatom is connected to W).

In another particular group of compounds of formula I, W is phenyl or 2- or 3-pyridyl substituted by trifluoromethyl. In one further group of compounds of formula I, W is phenyl or 2-substituted by trifluoromethyl.

In a further group of compounds of formula I, Y is CH₂.

In another group of compounds of formula I, Y is a bond.

In another group of compounds of formula I, Y is NH.

In yet another group of compounds of formula I, A is NH, X is a bond and Y is CH₂.

In a further particular group of compounds of formula I, A is C₁₋₄ alkyl, X is NH and Y is CH₂.

In another particular group of compounds of formula I, A is NH, X is a bond and Y is a bond.

In a further group of compounds of formula I, X is NH and Y is a bond.

In a further particular group of compounds of formula I, A is C₁₋₄ alkyl, X is NH and Y is a bond.

In another particular group of compounds of formula I, D represents (CH₂)ₙ, wherein n is 1 and E represents (CH₂)ₘ, wherein m is 1.

In another particular group of compounds of formula I, D represents (CH₂)ₙ, wherein n is 1 and E represents (CH₂)ₘ, wherein m is 0, or vice versa.

In another particular group of compounds of formula I, D represents (CH₂)ₙ, wherein n is 0 and E represents (CH₂)ₘ, wherein m is 0.

In another particular group of compounds of formula I, A represents pyrrolidinyl, piperidinyl, piperazinyl, or morpholinyl.

In another particular group of compounds of formula I, A represents piperidinyl.

The term “pharmaceutically acceptable salt” refers to pharmaceutically acceptable acid addition salts. A suitable pharmaceutically acceptable salt of a compound of Formula I is, for example, an acid-addition salt of a compound of Formula I which is sufficiently basic, for example an acid-addition salt with an inorganic or organic acid such as:
(1S)-(+)-10-camphorsulfonic acid; cyclohexylsulfaniic acid; phosphoric acid; diethylphosphoric acid; p-toluenesulfonic acid; L-lysine; L-lysine hydrochloride; saccharinic acid; methanesulfonic acid; hydrobromic acid; hydrochloric acid; sulphuric acid; 1,2-ethanedisulfonic acid; (+/−)-camphorsulfonic acid; ethanesulfonic acid; nitric acid; p-xylenesulfonic acid; 2-mesitylenesulfonic acid; 1,5-naphthalenedisulfonic acid; 1-naphthalenesulfonic acid; 2-naphthalenesulfonic acid; benzenesulfonic acid; maleic acid; D-glutamic acid; L-glutamic acid; D,L-glutamic acid; L-arginine; glycine; salicylic acid; tartaric acid; fumaric acid; citric acid; L-(−)-malic acid; D,L-malic acid and D-gluconic acid.

Throughout the specification and the appended claims, a given chemical formula or name shall encompass all tautomers, all stereo and optical isomers and racemates thereof as well as mixtures in different proportions of the separate enantiomers, where such isomers and enantiomers exist, as well as pharmaceutically acceptable salts thereof. Isomers may be separated using conventional techniques, e.g. chromatography or fractional crystallisation. The enantiomers may be isolated by separation of racemate for example by fractional crystallisation, resolution or HPLC. The diastereomers may be isolated by separation of isomer mixtures for instance by fractional crystallisation, HPLC or flash chromatography. Alternatively the stereoisomers may be made by chiral synthesis from chiral starting materials under conditions, which will not cause racemisation or epimerisation, or by derivatisation, with a chiral reagent. All stereoisomers are included within the scope of the invention.

Compounds of the present invention are intended to be chemically stable and it is assumed that it is within the skilled persons knowledge to identify which combinations of the above-defined groups in Formula I that may result in chemically unstable compounds of Formula 1.

The following definitions shall apply throughout the specification and the appended claims.

Unless otherwise stated or indicated, the term “alkyl” denotes either a straight chain or branched alkyl group. Examples of said alkyl include methyl, ethyl, n-propyl, isopropyl, cyclopropyl, n-butyl, iso-butyl, sec-butyl and t-butyl. Preferred alkyl groups are methyl, ethyl, propyl, isopropyl and tertiary butyl.

Unless otherwise stated or indicated, the term “alkenyl” denotes either a straight chain or branched alkenyl group wherein said group contains one or more double bonds.

Unless otherwise stated or indicated, the term “alkoxy” denotes a group O-alkyl, wherein alkyl is as defined above.

Unless otherwise stated or indicated, the term “halo” shall mean fluorine, chlorine, bromine or iodine

Specific compounds of the invention include one or more of the following:
2,2-diphenyl-N-[1-({1-[4-(trifluoromethyl)phenyl]-1H-pyrrol-3-yl}methyl)piperidin-4-yl]acetamide,
N-(3,4-difluorobenzyl)-2-[1-({1-[4-(trifluoromethyl)phenyl]-1H-pyrrol-3-yl}methyl)piperidin-4-yl]acetamide,
N-(2-phenylethyl)-1-({1-[4-(trifluoromethyl)phenyl]-1H-pyrrol-3-yl}methyl)piperidine-4-carboxamide,
N-[bis(4-fluorophenyl)methyl]-2-[1-({1-[5-(trifluoromethyl)pyridin-2-yl]-1H-pyrrol-3-yl}methyl)piperidin-4-yl]acetamide,
N,N-bis(4-fluorophenyl)-N-[1-({1-[5-(trifluoromethyl)pyridin-2-yl]-1H-pyrrol-3-yl}methyl)piperidin-4-yl]urea,
N-[bis(4-fluorophenyl)methyl]-2-[1-({1-[5-(trifluoromethyl)pyridin-2-yl]-1H-pyrrol-3-yl}methyl)pyrrolidin-3-yl]acetamide,
N-(4-fluorophenyl)-1-({1-[5-(trifluoromethyl)pyridin-2-yl]-1H-pyrrol-3-yl}methyl)piperidine-4-carboxamide acetate,
N-(1,3-benzothiazol-2-ylmethyl)-2-[1-({1-[4-(trifluoromethyl)phenyl]-1H-pyrrol-3-yl}methyl)piperidin-4-yl]acetamide,
N-(2-furylmethyl)-2-[1-({1-[4-(trifluoromethyl)phenyl]-1H-pyrrol-3-yl}methyl)piperidin-4-yl]acetamide,
N-(2-pyridin-2-ylethyl)-2-[1-({1-[4-(trifluoromethyl)phenyl]-1H-pyrrol-3-yl}methyl)piperidin-4-yl]acetamide,
N-(2,4-dichlorobenzyl)-2-[1-({1-[4-(trifluoromethyl)phenyl]-1H-pyrrol-3-yl}methyl)piperidin-4-yl]acetamide,
N-(1,2-diphenylethyl)-2-[1-({1-[4-(trifluoromethyl)phenyl]-1H-pyrrol-3-yl}methyl)piperidin-4-yl]acetamide
N-(1,3-benzodioxol-5-ylmethyl)-2-[1-({1-[4-(trifluoromethyl)phenyl]-1H-pyrrol-3-yl}methyl)piperidin-4-yl]acetamide,
N-ethyl-N-(2-pyridin-2-ylethyl)-2-[1-({1-[4-(trifluoromethyl)phenyl]-1H-pyrrol-3-yl}methyl)piperidin-4-yl]acetamide,
N-(2,3-dihydro-1,4-benzodioxin-2-ylmethyl)-2-[1-({1-[4-(trifluoromethyl)phenyl]-1H-pyrrol-3-yl}methyl)piperidin-4-yl]acetamide,
N-[3-(1H-imidazol-1-yl)propyl]-2-[1-({1-[4-(trifluoromethyl)phenyl]-1H-pyrrol-3-yl}methyl)piperidin-4-yl]acetamide,
N-(2,4-dichlorobenzyl)-2-[1-({1-[4-(trifluoromethyl)phenyl]-1H-pyrrol-3-yl}methyl)piperidin-4-yl]acetamide,
N-(4-fluorophenyl)-2-[1-({1-[4-(trifluoromethyl)phenyl]-1H-pyrrol-3-yl}methyl)piperidin-4-yl]acetamide,
N-[phenyl(pyridin-2-yl)methyl]-2-[1-({1-[4-(trifluoromethyl)phenyl]-1H-pyrrol-3-yl}methyl)piperidin-4-yl]acetamide,
N-[3-(difluoromethoxy)benzyl]-2-[1-({1-[4-(trifluoromethyl)phenyl]-1H-pyrrol-3-yl}methyl)piperidin-4-yl]acetamide,
1-(3-methoxyphenyl)-4-{[1-({1-[4-(trifluoromethyl)phenyl]-1H-pyrrol-3-yl}methyl)piperidin-4-yl]acetyl}piperazine,
1′-{[1-({1-[4-(trifluoromethyl)phenyl]-1H-pyrrol-3-yl}methyl)piperidin-4-yl]acetyl}spiro[indene-1,4′-piperidine],
N-(3,3-diphenylpropyl)-2-[1-({1-[4-(trifluoromethyl)phenyl]-1H-pyrrol-3-yl}methyl)piperidin-4-yl]acetamide,
N-(1-phenylpropyl)-2-[1-({1-[4-(trifluoromethyl)phenyl]-1H-pyrrol-3-yl}methyl)piperidin-4-yl]acetamide
N-(4-fluorophenyl)-N-methyl-2-[1-({1-[4-(trifluoromethyl)phenyl]-1H-pyrrol-3-yl}methyl)piperidin-4-yl]acetamide
N-[(1R,2S)-2-phenylcyclopropyl]-2-[1-({1-[4-(trifluoromethyl)phenyl]-1H-pyrrol-3-yl}methyl)piperidin-4-yl]acetamide,
N-(3-methylbutyl)-2-[1-({1-[4-(trifluoromethyl)phenyl]-1H-pyrrol-3-yl}methyl)piperidin-4-yl]acetamide,
N—[2-(3,4-dimethoxyphenyl)ethyl]-N-methyl-2-[1-({1-[4-(trifluoromethyl)phenyl]-1H-pyrrol-3-yl}methyl)piperidin-4-yl]acetamide
N-[2-(3,4-dimethoxyphenyl)ethyl]-2-[1-({1-[4-(trifluoromethyl)phenyl]-1H-pyrrol-3-yl}methyl)piperidin-4-yl]acetamide,
N-[4-(1,2,3-thiadiazol-4-yl)benzyl]-2-[1-({1-[4-(trifluoromethyl)phenyl]-1H-pyrrol-3-yl}methyl)piperidin-4-yl]acetamide,
N-[1-(2,3-dihydro-1,4-benzodioxin-5-yl)ethyl]-2-[1-({1-[4-(trifluoromethyl)phenyl]-1H-pyrrol-3-yl}methyl)piperidin-4-yl]acetamide,
N,N-diethyl-1-{[1-({1-[4-(trifluoromethyl)phenyl]-1H-pyrrol-3-yl}methyl)piperidin-4-yl}acetyl piperidine-3-carboxamide,
N-1-adamantyl-2-[1-({1-[4-(trifluoromethyl)phenyl]-1H-pyrrol-3-yl}methyl)piperidin-4-yl]acetamide,
N-[2-(4-methoxyphenoxy)ethyl]-2-[1-({1-[4-(trifluoromethyl)phenyl]-1H-pyrrol-3-yl}methyl)piperidin-4-yl]acetamide
N-{(1S)-1-[(benzyloxy)methyl]propyl}-2-[1-({1-[4-(trifluoromethyl)phenyl]-1H-pyrrol-3-yl}methyl)piperidin-4-yl]acetamide
N-[(1R)-1-(3-methoxyphenyl)ethyl]-2-[1-({1-[4-(trifluoromethyl)phenyl]-1H-pyrrol-3-yl}methyl)piperidin-4-yl]acetamide
N-{[3-(4-methoxyphenyl)isoxazol-5-yl]methyl}-2-[1-({1-[4-(trifluoromethyl)phenyl]-1H-pyrrol-3-yl}methyl)piperidin-4-yl]acetamide
4-(4-chlorophenyl)-1-{[1-({1-[4-(trifluoromethyl)phenyl]-1H-pyrrol-3-yl}methyl)piperidin-4-yl]acetyl}-1,2,3,6-tetrahydropyridine
N-{(1S,2S)-2-(benzyloxy)cyclopentyl]-2-[1-(1-[4-(trifluoromethyl)phenyl]-1H-pyrrol-3-yl}methyl)piperidin-4-yl]acetamide
N-(1-methyl-1-phenylethyl)-2-[1-({1-[4-(trifluoromethyl)phenyl]-1H-pyrrol-3-yl}methyl)piperidin-4-yl]acetamide
N-[(1-methyl-1H-pyrrol-2-yl)methyl]-2-[1-({1-[4-(trifluoromethyl)phenyl]-1H-pyrrol-3-yl}methyl)piperidin-4-yl]acetamide
4-(2-oxo-2-pyrrolidin-1-ylethyl)-1-({1-[4-(trifluoromethyl)phenyl]-1H-pyrrol-3-yl}methyl)piperidine,
N-(2-pyridin-2-ylethyl)-1-({1-[4-(trifluoromethyl)phenyl]-1H-pyrrol-3-yl}methyl)piperidine-4-carboxamide,
N-(2,4-dichlorobenzyl)-1-({1-[4-(trifluoromethyl)phenyl]-1H-pyrrol-3-yl}methyl)piperidine-4-carboxamide,
N-(1,2-diphenylethyl)-1-({1-[4-(trifluoromethyl)phenyl]-1H-pyrrol-3-yl}methyl)piperidine-4-carboxamide,
N-(1,3-benzodioxol-5-ylmethyl)-1-({1-[4-(trifluoromethyl)phenyl]-1H-pyrrol-3-yl}methyl)piperidine-4-carboxamide,
N-[2-(3,4-dimethoxyphenyl)ethyl]-1-({1-[4-(trifluoromethyl)phenyl]-1H-pyrrol-3-yl}methyl)piperidine-4-carboxamide,
N-[4-(1,2,3-thiadiazol-4-yl)benzyl]-1-({1-[4-(trifluoromethyl)phenyl]-1H-pyrrol-3-yl}methyl)piperidine-4-carboxamide,
N-(2,3-dihydro-1,4-benzodioxin-2-ylmethyl)-1-({1-[4-(trifluoromethyl)phenyl]-1H-pyrrol-3-yl}methyl)piperidine-4-carboxamide,
N-[1-(2,3-dihydro-1,4-benzodioxin-5-yl)ethyl]-1-({1-[4-(trifluoromethyl)phenyl]-1H-pyrrol-3-yl}methyl)piperidine-4-carboxamide,
N-[phenyl(pyridin-2-yl)methyl]-1-({1-[4-(trifluoromethyl)phenyl]-1H-pyrrol-3-yl}methyl)piperidine-4-carboxamide,
N-[3-(difluoromethoxy)benzyl]-1-({1-[4-(trifluoromethyl)phenyl]-1H-pyrol-3-yl}methyl)piperidine-4-carboxamide,
N-[2-(4-methoxyphenoxy)ethyl]-1-({1-[4-(trifluoromethyl)phenyl]-1H-pyrrol-3-yl}methyl)piperidine-4-carboxamide,
N—{(1S)-1-[(benzyloxy)methyl]propyl}-1-({1-[4-(trifluoromethyl)phenyl]-1H-pyrrol-3-yl}methyl)piperidine-4-carboxamide,
N-{[3-(4-methoxyphenyl)isoxazol-5-yl]methyl}-1-({1-[4-(trifluoromethyl)phenyl]-1H-pyrrol-3-yl}methyl)piperidine-4-carboxamide,
1-(3-methoxyphenyl)-4-{[1-({1-[4-(trifluoromethyl)phenyl]-1H-pyrrol-3-yl}methyl)piperidin-4-yl]carbonyl}piperazine,
4-(4-chlorophenyl)-1-{[1-(I{1-[4-(trifluoromethyl)phenyl]-1H-pyrrol-3-yl}methyl)piperidin-4-yl]carbonyl}-1,2,3,6-tetrahydropyridine,
N-[(1S,2S)-2-(benzyloxy)cyclopentyl]-1-({1-[4-(trifluoromethyl)phenyl]-1H-pyrrol-3-yl}methyl)piperidine-4-carboxamide,
N-(3,3-diphenylpropyl)-1-({1-[4-(trifluoromethyl)phenyl]-1H-pyrrol-3-yl}methyl)piperidine-4-carboxamide,
N-(1-phenylpropyl)-1-({1-[4-(trifluoromethyl)phenyl]-1H-pyrrol-3-yl}methyl)piperidine-4-carboxamide,
N-(1,3-benzothiazol-2-ylmethyl)-1-({1-[4-(trifluoromethyl)phenyl]-1H-pyrrol-3-yl}methyl)piperidine-4-carboxamide,
N-[(5-chloro-6-methoxypyridin-3-yl)(4-fluorophenyl)methyl]-2-[1-({1-[4-(trifluoromethyl)phenyl]-1H-pyrrol-3-yl}methyl)piperidin-4-yl]acetamide,
N-[(5-chloro-6-oxo-1,6-dihydropyridin-3-yl)(4-fluorophenyl)methyl]-2-[1-({1-[4-(trifluoromethyl)phenyl]-1H-pyrrol-3-yl}methyl)piperidin-4-yl]acetamide acetate salt,
N-(4-chloro-2-methoxybenzyl)-1-({1-[4-(trifluoromethyl)phenyl]-1H-pyrrol-3-yl}methyl)piperidine-4-carboxamide,
N-(4-chloro-2-hydroxybenzyl)-1-({1-[4-(trifluoromethyl)phenyl]-1H-pyrrol-3-yl}methyl)piperidine-4-carboxamide, acetate salt, 2-(3-fluorophenyl)-N-[1-({1-[4-(trifluoromethyl)phenyl]-1H-pyrrol-3-yl}methyl)piperidin-4-yl]pyrrolidine-1-carboxamide,
N-[2-(1H-imidazol-1-yl)-1-phenylethyl]-N′-[1-({1-[4-(trifluoromethyl)phenyl]-1H-pyrrol-3-yl}methyl)piperidin-4-yl]urea,
N-(3-fluorobenzyl)-N-methyl-N-[1-({1-[4-(trifluoromethyl)phenyl]-1H-pyrrol-3-yl}methyl)piperidin-4-yl]urea,
3-(1,1-dioxidothiomorpholin-4-yl)-N-[1-({1-[4-(trifluoromethyl)phenyl]-1H-pyrrol-3-yl}methyl)piperidin-4-yl]azetidine-1-carboxamide,
N-(3-hydroxybutyl)-N′-[1-({1-[4-(trifluoromethyl)phenyl]-1H-pyrrol-3-yl}methyl)piperidin-4-yl]urea,
N-[(1S)-2-hydroxy-1-phenylethyl]-N′-[1-({1-[4-(trifluoromethyl)phenyl]-1H-pyrrol-3-yl}methyl)piperidin-4-yl]urea,
2-(1,3-benzothiazol-2-yl)-N-[1-({1-[4-(trifluoromethyl)phenyl]-1H-pyrrol-3-yl}methyl)piperidin-4-yl]pyrrolidine-1-carboxamide,
2-(pyridin-3-ylmethyl)-N-[1-({1-[4-(trifluoromethyl)phenyl]-1H-pyrrol-3-yl}methyl)piperidin-4-yl]pyrrolidine-1-carboxamide,
(+)-2-(3-fluorophenyl)-N-[1-({1-[4-(trifluoromethyl)phenyl]-1H-pyrrol-3-yl}methyl)piperidin-4-yl]pyrrolidine-1-carboxamide,
(−)-2-(3-fluorophenyl)-N-[1-({1-[4-(trifluoromethyl)phenyl]-1H-pyrrol-3-yl}methyl)piperidin-4-yl]pyrrolidine-1-carboxamide,
(+)-N-[2-(1H-imidazol-1-yl)-1-phenylethyl]-N′-[1-({1-[4-(trifluoromethyl)phenyl]-1H-pyrrol-3-yl}methyl)piperidin-4-yl]urea,
(−)-N-[2-(1H-imidazol-1-yl)-1-phenylethyl]-N′-[1-({1-[4-(trifluoromethyl)phenyl]-1H-pyrrol-3-yl}methyl)piperidin-4-yl]urea,
2-(2-hydroxyethyl)-N-[1-({1-[4-(trifluoromethyl)phenyl]-1H-pyrrol-3-yl}methyl)piperidin-4-yl]piperidine-1-carboxamide;
N-(4-fluorobenzyl)-N-(3-hydroxypropyl)-N′-[1-({1-[4-(trifluoromethyl)phenyl]-1H-pyrrol-3-yl}methyl)piperidin-4-yl]urea;
N-(2-hydroxy-3-phenoxypropyl)-N′-[1-({1-[4-(trifluoromethyl)phenyl]-1H-pyrrol-3-yl}methyl)piperidin-4-yl]urea;
N-[(1-hydroxycyclohexyl)methyl]-N′-[1-({1-[4-(trifluoromethyl)phenyl]-1H-pyrrol-3-yl}methyl)piperidin-4-yl]urea;
N-[(4-fluorophenyl)(6-methoxypyridin-3-yl)methyl]-2-[1-({1-[4-(trifluoromethyl)phenyl]-1H-pyrrol-3-yl}methyl)piperidin-4-yl]acetamide;
N-[(4-fluorophenyl)(6-oxo-1,6-dihydropyridin-3-yl)methyl]-2-[1-({1-[4-(trifluoromethyl)phenyl]-1H-pyrrol-3-yl}methyl)piperidin-4-yl]acetamide; and
N-[2-(2-methyl-1H-imidazol-1-yl)-1-phenylethyl]-N′-[1-({1-[4-(trifluoromethyl)phenyl]-1H-pyrrol-3-yl}methyl)piperidin-4-yl]urea;
   and pharmaceutically acceptable salts thereof.

In one further aspect of the invention, there is provided N-[4-(trifluoromethoxy)phenyl]-N′-[1-({1-[4-(trifluoromethyl)phenyl]-1H-pyrrol-3-yl}methyl)piperidin-4-yl]urea.

In yet one further aspect of the invention, there is provided N-(2,4-dichlorophenyl)-N′-[1-({1-[4-(trifluoromethyl)phenyl]-1H-pyrrol-3-yl}methyl)piperidin-4-yl]urea, and pharmaceutically acceptable salts thereof.

In another aspect of the invention, there is provided N-1-naphthyl-N′-[1-({1-[4-(trifluoromethyl)phenyl]-1H-pyrrol-3-yl}methyl)piperidin-4-yl]urea, and pharmaceutically acceptable salts thereof.

In yet another aspect of the invention, there is provided N-(3-fluorobenzyl)-N′-[1-({1-[4-(trifluoromethyl)phenyl]-1H-pyrrol-3-yl}methyl)piperidin-4-yl]urea, and pharmaceutically acceptable salts thereof.

In one further aspect of the invention, there is provided N-(diphenylmethyl)-1′-[1-({1-[4-(trifluoromethyl)phenyl]-1H-pyrrol-3-yl}methyl)piperidin-4-yl]urea, and pharmaceutically acceptable salts thereof.

In another aspect of the invention, there is provided N-methyl-N-phenyl-N-[1-({1-[5-(trifluoromethyl)pyridin-2-yl]-1H-pyrrol-3-yl}methyl)piperidin-4-yl]urea and pharmaceutically acceptable salts thereof.

Another particular group of Formula I comprises compounds in which A is C₁ alkyl, X is N—H and Y is NiH.

### Methods of Preparation

The compounds of the invention may be prepared as outlined below according to any of the following methods. However, the invention is not limited to these methods, the compounds may also be prepared as described for structurally related compounds in the prior art.

Compounds of formula I may be prepared by reacting a compound of formula II

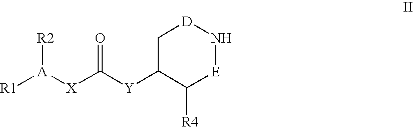

in which R¹, R², R⁴, A, X, Y, D, and E are as previously defined,
with a compound of formula III

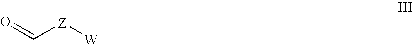

in which Z and W are as previously defined.

For example, a compound of formula II and a compound of formula III may be reacted together at a temperature in the range of 0° C. to 150° C., preferably iii the range of 20° C. to 80° C. in the presence of a solvent, for example methanol, DCM, CHCl₃, THF or dioxane, in the presence of a reducing agent, for example sodium cyanoborohydride (optionally polymer supported) or sodium triacetoxyborohydride (optionally polymer supported). Optionally, a catalytic amount of an acid, e.g. acetic acid, may be added to the reaction mixture.

Alternatively, compounds of formula I, in which Y represents NR³, may be prepared by reacting a compound of formula IV,

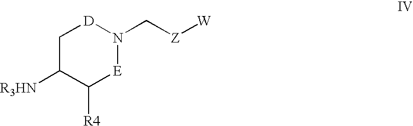

in which R³, R⁴, D, E, Z and W are as previously defined, with a compound of formula V

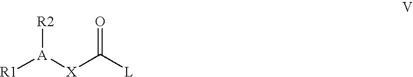

in which R¹, R², A and X are as previously defined, L represents a leaving group such as chloride or (provided that A-X does not represent N) a hydroxy group.

For example, a compound of formula IV and a compound of formula V, in which L is a hydroxy group, may be reacted together at a temperature in the range of 0° C. to 150° C., preferably in the range of 20° C. to 80° C. in the presence of a solvent, for example THF, DCM, DCM/water (i.e. a two phase system) or DMF, optionally in the presence of a suitable inorganic or organic base, e.g. DIPEA or TEA, and a standard amide coupling reagent, e.g. HATU, TBTU, TFFFH, PyBroP, EDC, or DCC, the latter two of which may optionally be polymer supported. Suitable additives such as HOBt and HOAt may also be optionally utilised.

Alternatively, compounds of formula I may be obtained by reaction of compounds of formula IV with compounds of formula V, in which L is chloride, in an inert solvent, e.g. THF, dioxane, DCM, CHCl₃ or 1,2-dichloroethane in the presence of a suitable inorganic or organic base, e.g. DIPEA, TEA, collidine, K₂CO₃ or NaHCO₃.

Alternatively, compounds of formula I in which A=C and X═N, or in which A=N and

X=bond, Y represents NR³ and in which R² is hydrogen, may be prepared by reacting a compound of formula IV, with a compound of formula VI

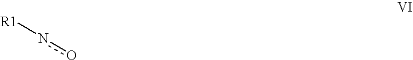

in which R¹ is as previously defined (provided that R¹ is not H)

For example, a compound of formula IV and a compound of formula VI may be reacted together at a temperature in the range of 20° C. to 80° C. in the presence of a dry, inert solvent, for example THF or DCM, optionally in the presence of a suitable inorganic or organic base, e.g. DIPEA or TEA.

Alternatively, compounds of formula I, in which A represents a C₁₋₄ alkyl group (straight chain or branched) and X represents NR³, or in which A represents N and X represents a bond, and in which Y represents a bond or a C(R³)₂ group, may be prepared by reacting a compound of formula VII,

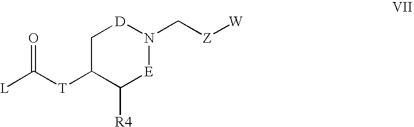

in which R⁴, D, E, Z and W are as previously defined, L is a hydroxy group or a leaving group such as chloride or fluoride and in which T represents a bond or a C(R³)₂-2-group with a compound of formula VIII,

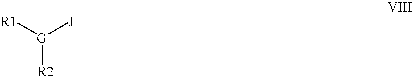

in which G represents N and J represents H, or in which G represents a C₁₋₄ alkyl group (straight chain or branched) and J represents NR³, and in which R¹, R² and R³ are as previously defined,

For example, a compound of formula VII and a compound of formula VIII, in which L is a hydroxy group, may be reacted together at a temperature in the range of 0° C. to 150° C., preferably in the range of 20° C. to 80° C. in the presence of a solvent, for example THF, DCM, DCM/water (i.e. a two phase system) or DME, optionally in the presence of a suitable inorganic or organic base, e.g. DIPEA or TEA, and a standard amide coupling reagent, e.g. HATU, TBTU, TFFH, PyBroP, EDC, or DCC, the latter two of which may optionally be polymer supported. Suitable additives such as HOBt and HOAt may also be optionally utilised.

Alternatively, compounds of formula I may be obtained by reaction of compounds of formula VII, in which L is chloride, with compounds of formula VIII in an inert solvent, e.g. TEF, dioxane, DCM, CHCl₃ or 1,2-dichloroethane in the presence of a suitable inorganic or organic base, e.g. DIPEA, TEA, collidine, K₂CO₃ or NaHCO₃.

Using methods similar to those described hereinbefore, compounds of formula II, in which B represents NR³, may be prepared by reaction of compounds of formula IX with compounds of formula V or VI

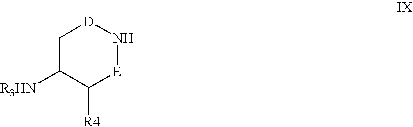

Alternatively, using methods similar to those described hereinbefore, compounds of formula II, in which Y represents a bond or a C(R³)₂ group, may be prepared by reacting a compound of formula X in which R⁴, D, E, L and T are as previously defined

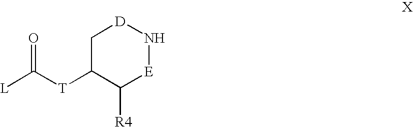

with a compound of formula VIII

Compounds of formula III, in which Z is a 1H-pyrrol-3-yl ring, may be prepared by reaction of a compound of formula XI with a compound of formula XII in which W is as previously defined.

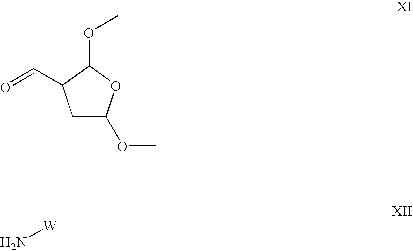

For example, a compound of formula XI and a compound of formula XII may be reacted together at a temperature in the range of 20° C. to 90° C. in acetic acid.

Alternatively, compounds of formula III, in which Z is a 1H-pyrrol-3-yl ring, may be prepared by reaction of a compound of formula XII with a compound of formula XIV in which W is as previously defined and in which U is chloride or a bromide

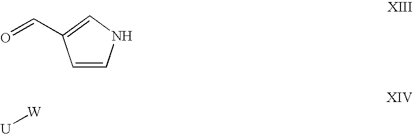

For example, a compound of formula XV and a compound of formula XVI may be reacted together in an inert solvent such as THF or dioxane in the presence of a strong base, e.g. NaH, at a temperature in the range of 20° C. to 60° C.

Alternatively, compounds of formula III may be prepared by reaction of a compound of formula XV, in which Z is as previously defined and in which V is bromide or iodide with a compound of formula XVI in which W is as previously defined.

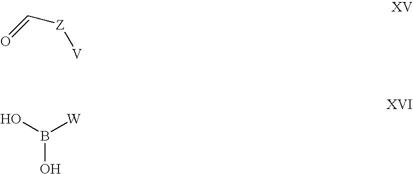

For example, a compound of formula XV and a compound of formula XVI may be reacted together under palladium catalysis using a method described e.g. in Feuerstein, M. et al., Tetrahedron Lett. 42 (33), 5659, 2001.

Alternatively, using similar synthetic methodology, compounds of formula III may be prepared by reaction of a compound of formula XVII, in which Z is as previously defined with a compound of formula XVIII in which W and V are as previously defined

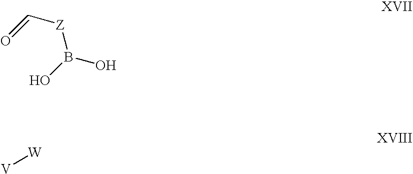

Using methods similar to those hereinbefore described, compounds of formulae IV and VII may be prepared by reaction of compounds of formulae IX and X respectively, with a compound of formula III.

Compounds of formula IX and X, in which R⁴ represents a fluorine atom (and D and E are both representing CH₂) may be prepared starting with fluorination (using eg. SELECTFLUOR™ Reagent) of the silyl enol ether of piperidone, as described e.g. by van Neil, M. B. et al. J. Med. Chem. 1999, 42, 2087-2104. Reductive amination of the so formed α-fluoro piperidone gives compounds of formula IX. Preparation of compounds of formula X, where T represents CH₂, from α-fluoro piperidone may be carried out by standard methods, e.g. as described in PCT Int. Appl. WO2001000206. Additionally, compounds of formula X, where T represents a bond, could conceivably be prepared in analogy to chemistry described e.g. by Borne, R. F. et al. J. Heterocyclic Chemistry (1990), 27(2), 375-84.

Compounds of formula III, V, VI and VIII-XVIII are either commercially available or can be prepared by methods well known to those skilled in the art, e.g. as described hereinafter in the Experimental Section.

Optionally, the nitrogen of the ring in formulae IX an X may be protected prior to reaction with a compound of formula V and VIR. Amine protecting groups are known to those skilled in the art, for example the benzyl, t-Boc, or Cbz groups.

Optionally, the carboxylic acid in compounds of formula X may be protected as an ester prior to reaction with a compound of formula III. Suitable esters are e.g. ethyl, tert-butyl or benzyl esters.

The compounds of the invention may be isolated from their reaction mixtures using conventional techniques. Stereoisomers may be separated using conventional techniques, e.g. chromatography or fractional crystallisation. Enantiomers may be isolated by separation of racemate for example by fractional crystallisation, resolution or HPLC. The diastereomers may be isolated by separation of isomer mixtures for instance by fractional crystallisation, HPLC or flash chromatography. Alternatively the stereoisomers may be made by chiral synthesis from chiral starting materials under conditions which will not cause racemisation or epimerisation, or by derivatisation, with a chiral reagent.

Persons skilled in the art will appreciate that, in order to obtain compounds of the invention in an alternative and in some occasions, more convenient manner, the individual process steps mentioned hereinbefore may be performed in a different order, and/or the individual reactions may be performed at a different stage in the overall route (i.e. chemical transformations may be performed upon different intermediates to those associated hereinbefore with a particular reaction).

### Pharmaceutical Preparations

The compounds of the invention will normally be administered via the oral, parenteral, intravenous, intramuscular, subcutaneous or in other injectable ways, buccal, rectal, vaginal, transdermal and/or nasal route and/or via inhalation, in the form of pharmaceutical preparations comprising the active ingredient either as a free base, or a pharmaceutically acceptable inorganic or organic addition salt, in a pharmaceutically acceptable dosage form. Depending upon the disorder and patient to be treated and the route of administration, the compositions may be administered at varying doses.

Suitable daily doses of the compounds of the invention in the therapeutic treatment of humans are about 0.001-10 mg/kg body weight, preferably 0.01-3 mg/kg body weight.

Oral formulations are preferred particularly tablets or capsules which may be formulated by methods known to those skilled in the art to provide doses of the active compound in the range of 0.5 mg to 500 mg for example 1 mg, 3 mg, 5 mg, 10 mg, 25 mg, 50 mg, 100 mg and 250 mg.

According to a further aspect of the invention there is also provided a pharmaceutical formulation including any of the compounds of the invention, or pharmaceutically acceptable derivatives thereof, in admixture with pharmaceutically acceptable adjuvants, diluents and/or carriers.

The compounds of the invention may also be combined with other therapeutic agents, which are useful in the treatment of disorders associated with obesity, psychiatric disorders, neurological disorders and pain.

### Pharmacological Properties

The compounds of formula (I) are useful for the treatment of obesity, psychiatric disorders such as psychotic disorders, anxiety, anxio-depressive disorders, depression, cognitive disorders, memory disorders, schizophrenia, epilepsy, and related conditions, and neurological disorders such as dementia, multiple sclerosis, Raynaud's syndrome, Parkinson's disease, Huntington's chorea and Alzheimer's disease. The compounds are also potentially useful for the treatment of immune, cardiovascular, reproductive and endocrine disorders, and diseases related to the respiratory and gastrointestinal systems. The compounds are also potentially useful as agents for ceasing consumption of tobacco, treating nicotine dependence and/or treating nicotine withdrawal symptoms, reducing the craving for nicotine and as anti-smoking agents. The compounds may also eliminate the increase in weight that normally accompanies the cessation of smoking. The compounds are also potentially useful as agents for treating or preventing diarrhea.

The compounds are also potentially useful as agents for reducing the craving/relapse for addictive substances that include, but are not limited to psychomotor-active agents such as nicotine, alcohol, cocaine, amphetamines, opiates, benzodiazepines and barbiturates. The compounds are also potentially useful as agents for treating drug addiction and/or drug abuse.

Accordingly, it is desirable to provide a compound and method of treatment which will be active in reducing craving for the abused substance, and which does not exacerbate the sympathetic response rate caused by the abused substance and which has favourable pharmacodynamic effects.

The compounds are also potentially useful as agents for treating pain disorders, including but not limited to acute and chronic nociceptive, inflammatory and neuropathic pain and migraine.

In another aspect the present invention provides a compound of formula I as claimed in any previous claim for use as a medicament.

In a further aspect the present invention provides the use of a compound of formula I in the preparation of a medicament for the treatment or prophylaxis of obesity, psychiatric disorders such as psychotic disorders, anxiety, anxtio-depressive disorders, depression, bipolar disorder, ADHD, cognitive disorders, memory disorders, schizophrenia, epilepsy, and related conditions, neurological disorders such as dementia, multiple sclerosis, Parkinson's disease, Huntington's chorea and Alzheimer's disease and pain related disorders, including but not limited to acute and chronic nociceptive, inflammatory and neuropathic pain and migraine, comprising administering a pharmacologically effective amount of a compound of formula I to a patient in need thereof.

In a still further aspect the present invention provides a method of treating obesity, psychiatric disorders such as psychotic disorders, anxiety, anxio-depressive disorders, depression, bipolar disorder, ADHD, cognitive disorders, memory disorders, schizophrenia, epilepsy, and related conditions, and neurological disorders such as dementia, multiple sclerosis, Parkinson's disease, Huntington's chorea and Alzheimer's disease and pain related disorders, including but not limited to acute and chronic nociceptive, inflammatory and neuropathic pain and migraine, comprising administering a pharmacologically effective amount of a compound of formula I to a patient in need thereof.

The compounds of the present invention are particularly suitable for the treatment of obesity.

In another aspect the present invention provides a method of treating obesity, type II diabetes, Metabolic syndrome and a method of preventing type II diabetes comprising administering a pharmacologically effective amount of a compound of formula I to a patient in need thereof.

### Combination Therapy

The compounds of the invention may be combined with another therapeutic agent that is useful in the treatment of disorders associated with the development and progress of atherosclerosis such as hypertension, hyperlipidaemias, dyslipidaemias, diabetes and obesity. For example, a compound of the present invention may be used in combination with a compound that affects thermogenesis, lipolysis, fat absorption, satiety, or gut motility. The compounds of the invention may be combined with another therapeutic agent that decreases the ratio of LDL:HDL or an agent that causes a decrease in circulating levels of LDL-cholesterol. In patients with diabetes mellitus the compounds of the invention may also be combined with therapeutic agents used to treat complications related to micro-angiopathies.

The compounds of the invention may be used alongside other therapies for the treatment of metabolic syndrome or type 2 diabetes and its associated complications; these include biguanide drugs, insulin (synthetic insulin analogues), oral antihyperglycemics (these are divided into prandial glucose regulators and alpha-glucosidase inhibitors) and PPAR modulating agents.

In another aspect of the invention, the compound of formula I, or a pharmaceutically acceptable salt, solvate, solvate of such a salt or a prodrug thereof, may be administered in association with a PPAR modulating agent. PPAR modulating agents include but are not limited to a PPAR alpha and/or gamma agonist, or pharmaceutically acceptable salts, solvates, solvates of such salts or prodrugs thereof. Suitable PPAR alpha and/or gamma agonists, pharmaceutically acceptable salts, solvates, solvates of such salts or prodrugs thereof are well known in the art.

In addition the combination of the invention may be used in conjunction with a sulfonylurea. The present invention also includes a compound of the present invention in combination with a cholesterol-lowering agent. The cholesterol-lowering agents referred to in this application include but are not limited to inhibitors of HMG-CoA reductase (3-hydroxy-3-methylglutaryl coenzyme A reductase). Suitably the HMG-CoA reductase inhibitor is a statin.

In the present application, the term “cholesterol-lowering agent” also includes chemical modifications of the HMG-CoA reductase inhibitors, such as esters, prodrugs and metabolites, whether active or inactive.

The present invention also includes a compound of the present invention in combination with an inhibitor of the ileal bile acid transport system (IBAT inhibitor). The present invention also includes a compound of the present invention in combination with a bile acid binding resin.

According to an additional further aspect of the present invention there is provided a combination treatment comprising the administration of an effective amount of a compound of the formula I, or a pharmaceutically acceptable salt, solvate, solvate of such a salt or a prodrug thereof, optionally together with a pharmaceutically acceptable diluent or carrier, with the simultaneous, sequential or separate administration one or more of the following agents selected from:

a CETP (cholesteryl ester transfer protein) inhibitor;
a cholesterol absorption antagonist;
a MTP (microsomal transfer protein) inhibitor;
a nicotinic acid derivative, including slow release and combination products;
a phytosterol compound;
probucol;
an anti-obesity compound, for example orlistat (EP 129,748) and sibutramine (GB 2,184,122 and U.S. Pat. No. 4,929,629);
an antihypertensive compound, for example an angiotensin converting enzyme (ACE) inhibitor, an angiotensin II receptor antagonist, an andrenergic blocker, an alpha andrenergic blocker, a beta andrenergic blocker, a mixed alpha/beta andrenergic blocker, an andrenergic stimulant, calcium channel blocker, an AT-1 receptor blocker, a saluretic, a diuretic or a vasodilator;
a CB1 antagonist or inverse agonist, for example rimonabant;
another melanin concentrating hormone receptor 1 (MC1-r1) antagonist;
a PDK inhibitor; or
modulators of nuclear receptors for example LXR, FXR, RxR, and RORalpha;

### an SSRI;

a serotonin antagonist;
or a pharmaceutically acceptable salt, solvate, solvate of such a salt or a prodrug thereof, optionally together with a pharmaceutically acceptable dilutent or carrier to a warm-blooded animal, such as man in need of such therapeutic treatment.

Therefore in an additional feature of the invention, there is provided a method for the treatment of type 2 diabetes and its associated complications in a warm-blooded animal, such as man, in need of such treatment which comprises administering to said animal an effective amount of a compound of formula I, or a pharmaceutically acceptable salt, solvate, solvate of such a salt or a prodrug thereof in simultaneous, sequential or separate administration with an effective amount of a compound from one of the other classes of compounds described in this combination section, or a pharmaceutically acceptable salt, solvate, solvate of such a salt or a prodrug thereof.

Therefore in an additional feature of the invention, there is provided a method of treating hyperlipidemic conditions in a warm-blooded animal, such as man, in need of such treatment which comprises administering to said animal an effective amount of a compound of formula I, or a pharmaceutically acceptable salt, solvate, solvate of such a salt or a prodrug thereof in simultaneous, sequential or separate administration with an effective amount of a compound from one of the other classes of compounds described in this combination section or a pharmaceutically acceptable salt, solvate, solvate of such a salt or a prodrug thereof.

According to a further aspect of the invention there is provided a pharmaceutical composition which comprises a compound of formula I, or a pharmaceutically acceptable salt, solvate, solvate of such a salt or a prodrug thereof, and a compound from one of the other classes of compounds described in this combination section or a pharmaceutically acceptable salt, solvate, solvate of such a salt or a prodrug thereof, in association with a pharmaceutically acceptable diluent or carrier.

According to a further aspect of the present invention there is provided a kit comprising a compound of formula I, or a pharmaceutically acceptable salt, solvate, solvate of such a salt or a prodrug thereof, and a compound from one of the other classes of compounds described in this combination section or a pharmaceutically acceptable salt, solvate, solvate of such a salt or a prodrug thereof.

According to a further aspect of the present invention there is provided a kit comprising:

a) a compound of formula I, or a pharmaceutically acceptable salt, solvate, solvate of such a salt or a prodrug thereof, in a first unit dosage form;
b) a compound from one of the other classes of compounds described in this combination section or a pharmaceutically acceptable salt, solvate, solvate of such a salt or a prodrug thereof; in a second unit dosage form; and
c) container means for containing said first and second dosage forms.

According to a further aspect of the present invention there is provided a kit comprising:

a) a compound of formula I, or a pharmaceutically acceptable salt, solvate, solvate of such a salt or a prodrug thereof, together with a pharmaceutically acceptable diluent or carrier, in a first unit dosage form;
b) a compound from one of the other classes of compounds described in this combination section or a pharmaceutically acceptable salt, solvate, solvate of such a salt or a prodrug thereof, in a second unit dosage form; and
c) container means for containing said first and second dosage forms.

According to another feature of the invention there is provided the use of a compound of the formula I, or a pharmaceutically acceptable salt, solvate, solvate of such a salt or a prodrug thereof, and one of the other compounds described in this combination section, or a pharmaceutically acceptable salt, solvate, solvate of such a salt or a prodrug thereof, in the manufacture of a medicament for use in the treatment of metabolic syndrome or type 2 diabetes and its associated complications in a warm-blooded animal, such as man.

According to another feature of the invention there is provided the use of a compound of the formula I, or a pharmaceutically acceptable salt, solvate, solvate of such a salt or a prodrug thereof, and one of the other compounds described in this combination section, or a pharmaceutically acceptable salt, solvate, solvate of such a salt or a prodrug thereof, in the manufacture of a medicament for use in the treatment of hyperlipidaemiic conditions in a warm-blooded animal, such as man.

According to a further aspect of the present invention there is provided a combination a treatment comprising the administration of an effective amount of a compound of the formula I, or a pharmaceutically acceptable salt, solvate, solvate of such a salt or a prodrug thereof, optionally together with a pharmaceutically acceptable diluent or carrier, with the simultaneous, sequential or separate administration of an effective amount of one of the other compounds described in this combination section, or a pharmaceutically acceptable salt, solvate, solvate of such a salt or a prodrug thereof, optionally together with a pharmaceutically acceptable diluent or carrier to a warm-blooded animal, such as man in need of such therapeutic treatment.

### EXPERIMENTAL SECTION

The invention will now be described in more detail with the following examples that are not to be construed as limiting the invention.

### Abbreviations:

aq. aqueous
Ac acetyl
Bu butyl
tBoc tert-butyloxycarbonyl
Cbz benzyloxycarbonyl
CHO Chinese hamster ovary (cells)
DCM methylene chloride, CH₂Cl₂
DIPEA N,N-Diisopropylethylamine
DMA dimethyl acetamide
DMF N,N-dimethylformamide
DTT dithiothreitol
EDC 1-Ethyl-3-(3-dimethylaminopropyl)carbodiimide hydrochloride
EDTA ethylenediamine tetraacetic acid
ELS evaporative light scattering
ESI electrospray ionization
Et ethyl
GDP guanosine 5′-diplhosphate
GPCR G-protein coupled receptor
GTP guanosine 5′-triphosphate
HATU O-(azabenzotriazol-1-yl)-N,N, N′,N′-tetramethyluronium hexafluoro-phosphate
HEK human embryotic kidney (cells)
HEPES N-2-hydroxyethyl piperazine-N′-2-ethanesulfonic acid
hERG human ether-a-go-go related gene (potassium ion channel)
HPLC high performance liquid chromatography
HOAt 1-Hydroxy-7-azabenzotriazole
LC liquid chromatography
MP-BH(OAc)₃: macroporous polymer bound triacetoxyborohydride (available from Argonaut) Typically 2-3 meq/g BH
MS mass spectroscopy
Pol-BH₃CN (polystyrylmethyl)trimethylammonium cyanoborohydride (loading 4.1-4.3 mmol BH₃CN/g)
Pol-CHO 4-benzyloxybenzaldehyde polystyrene (loading ˜2.66 mmol CHO/g)
PyBroP Bromo-tris-pyrrolidino-phosphonium hexafluorophosphate
TBTU N,N, N′,N′-tetramethyl-O-(benzotriazol-1-yl)uronium tetrafluoroborate
TEA triethylamine
TFA trifluoroacetic acid
TFFH tetramethylfluoroformamidium hexafluorophosphate
THF tetrahydrofuran
TLC thin layer chromatography
Tris trishydroxymethylaminomethane
Tween polyoxyethylene sorbitan monolaurate
t tert
rt. room temperature
sat. saturated
br broad
bs broad singlet
d doublet
dd doublet of doublets
dt doublet of triplets
m multiplet
q quartet
singlet
t triplet

### General Experimental Procedures

Flash column chromatography employed MERCK normal phase silica gel 60 Å (40-63 μm) or a Biotage Horizon Pioneer® HPFC system equipped with FLASH 12+M or FLASH 25+M or 40+M silica cartridges. Mass spectra were recorded on a Waters Micromass ZQ single quadrupole equipped with a pneumatically assisted electrospray interface (LC-MS).

HPLC analyses were performed on a Gynkotek P580 HPG, gradient pump with a Gynkotek UVD 170S UV-Vis detector. Column: Chromolith Performance RP-18e, 4.6×100 mm, Mobile phase A: Acetonitrile, Mobile phase B: 0.1% TFA (aq), Flow: 3 ml/min, Injection volume: 20 μl, Detection: 254 and 275 nm.

Purifications were performed on a semi preparative HPLC, Shimadzu LC-8A, Shimadzu SPD-10A UV-vis. detector equipped with a Waters X-terra® Prep MS C₁₈ Column, 250 nmm×50 mm (10 μm) or on a Waters Prep LC 2000 with UV-detection, equipped with a Kromasil 10 μm C8 250 mm×20 mm column, or on a semi preparative HPLC, Shimadzu LC-8A, Shimadzu SPD-10A UV-vis.-detector equipped with a Waters Symmetry® 100 mm×19 mm C18 5 μm column.

Automated Preparative HPLC was done using a Waters Fraction Lynx system equipped with UV, ELS and MS detection and an Ace C8 5μ, 10 cm×21,2 id column. The mobile phase was A: 95% CH₃CN and B: 5% CH₃CN+95% 0.1 M NH₄OAc with a gradient from 100% B to 100% A in 10 minutes at 25 mL/min flow rate.

¹H NMR and ¹³C NMR spectra were obtained at 298 K on a Varian Unity Plus 400 mHz, or a Varian Inova 500 MHz or a Varian Unity Plus 600 MHz or a Bruker Avance 300 MHz or Varian Gemini 2000 300 MHz. Chemical shifts are given in ppm with the solvent residual peak as internal standard: CDCl₃ δ_{H} 7.26, δ_{C} 77.2; MeOH-d₄ δ_{H} 3.31, δ_{C} 49.0; DMSO-d₆ δ_{H} 2.50; δ_{C} 39⁻⁵ ppm.

Microwave heating was performed using single node heating in a Smith Creator from Personal Chemistry, Uppsala, Sweden.

Chemical names (IUPAC) were generated using the software ACD/Name version 7.00. Names/reference numbers of starting materials (CAS no), either commercially available or prepared according to literature procedures.

Pyrrol-3-aldehyde, 7126-39-8; 2-chloro-5-(trifluoromethyl)-pyridine, 52334-81-3; 2,5-dimethoxy-3-tetrahydrofurancarboxaldehyde, 50634-05-4; 4-aminobenzotrifluoride, 455-14-1; 5-trifluoromethyl-pyridine-2-ylamine, 74784-70-6; tert-butyl piperidin-4-ylcarbamate 73874-95-0; bis(4-fluorophenyl)methanone, 345-92-6; [1-(tert-butoxycarbonyl)piperidin-4-yl]acetic acid, 157688-46-5; (3,4-difluorobenzyl)amine, 72235-53-1; 1-(tert-butoxycarbonyl)piperidine-4-carboxylic acid, 84358-13-4; diphenylacetic acid, 117-34-0; 1-isocyanato-4-(trifluoromethoxy)benzene, 35037-73-1; 2,4-dichloro-1-isocyanatobenzene, 2612-57-9; 1-isocyanatonaphthalene, 86-84-0; 1-fluoro-3-(isocyanatomethyl)benzene, 102422-56-0; 4-fluoroaniline, 371-40-4; 1-bromo-4-fluorobenzene, 460-00-4; tert-buty 4-aminopiperidine-1-carboxylate, 87120-72-7; tert-butyl piperidin-4-ylcarbamate, 73874-95-0; ethyl piperidine-4-carboxylate, 1126-09-6; (4-fluorophenyl)amine, 371-40-4; 1-(tert-butoxycarbonyl)pyrrolidin-3-yl]acetic acid, 175526-97-3; methyl(phenyl)carbamic chloride, 4285-42-1; 1-(1,3-benzothiazol-2-yl)methanamine hydrochloride, 29198-41-2; 2-(3-fluorophenyl)pyrrolidine, 298690-72-9; 2-(1H-imidazol-1-yl)-1-phenylethanamine 24169-72-0; (3-fluorobenzyl)methylaamine, 90389-84-7; 4-azetidin-3-ylthiomorpholine 1,1-dioxide, 780732-40-3; 4-aminobutan-2-ol, 39884-48-5; (2S)-2-amino-2-phenylethanol, 7568-92-5; 2-pyrrolidin-2-yl-1,3-benzothiazole, 359804-21-0; 3-(pyrrolidin-2-ylmethyl)pyridine, 106366-28-3; 4-chloro-2-methoxybenzoic acid, 57479-70-6; [1-(tert-butoxycarbonyl)piperidin-4-yl]acetic acid, 157688-46-5; 2-piperidin-2-ylethanol, 1484-84-0; 3-[(4-fluorobenzyl)amino]propan-1-ol, 161798-73-8; 1-amino-3-phenoxypropan-2-ol hydrochloride, 86809-29-2; 1-(aminomethyl)cyclohexanol hydrochloride, 19968-85-5; methyl 6-methoxynicotinate, 26218-80-4; 2-methyl-1H-imidazole, 693-98-1; 2-bromo-1-phenylethanone, 70-11-1.

5-chloro-6-methoxynicotinic acid (cat. no. 111823) was purchased from Asymchem Laboratiories, Inc., Durham, N.C., USA.

### Preparation of Intermediates

### Example A

### 1-[4-(trifluoromethyl)phenyl]-1H-pyrrole-3-carbaldehyde

To a solution of 2,5-dimethoxy-3-tetrahydrofurancarboxaldehyde (8.0 g, 49.9 mmol) in acetic acid (120 mL) was added 4-aminobenzotrifluoride (8.05 g, 49.9 mmol) and the mixture was heated at reflux under an atmosphere of nitrogen until IPLC indicated that starting material was consumed. The reaction mixture was concentrated and the residue was dissolved in EtOAc (500 m-L) and washed with 2M NaOH (aq) (100 mL) and brine. The organic phase was dried (Na₂SO₄) and then evaporated to dryness. The residue was purified on SiO₂ eluted with DCM and finally DCM:MeOH (98:2) to give 8.56 g (72%) of the title compound (94% pure, HPLC purity).

¹H NMR (CDCl₃) δ 9.87 (s, 1H), 7.76 (m, 2H), 7.72 (m, 1H), 7.55 (m, 2H), 7.14 (m, 1H), 6.84 (m, 1H).

¹³C NMR (CDCl₃) δ 185.5, 142.2, 129.4 (q, J=33 Hz), 129.0, 127.4 (q, J=4 Hz), 126.8, 123.8 (q, J=272 Hz), 122.1, 121.1, 110.5.

MS (ESI) 240 (M+1H⁺).

### Example B

### 1-[5-(trifluoromethyl)pyridin-2-yl]-1H-pyrrole-3-carbaldehyde

Pyrrol-3-aldehyde (4.0 g, 42.1 mmol) in TEF (100 mL) was added to NaH (1.5 g, 63.2 mmol) in THF (30 mL) and the mixture was stirred at r.t. under an atmosphere of nitrogen until no more H₂ was generated. 2-chloro-5-(trifluoromethyl)-pyridine (8.4 g, 46.3 mmol) was added and the mixture was stirred at 50° C. under an atmosphere of nitrogen for 75 minutes. The solvent was removed by evaporation and water was added to the resulting solid. The aq. layer was washed with DCM and the organic layer was separated and dried over Na₂SO₄. The resulting brown residue was purified twice on a SiO₂ column eluting first with pure DCM and then with Heptane:EtOAc (3:1). The resulting yellow solid was washed with cold Et₂O to give 5.73 g (57%) of the title compound as a solid.

MS (ESI) 241 (M+H⁺).

### Example C

### 1-({1-[4-(trifluorginetyl)phenyl]-1H-pyrrol-3-yl}methyl)piperidin-4-amine dihydrochloride

### a) tert-butyl-[1-({1-[4-(trifluoromethyl)phenyl]-1H-pyrrol-3-yl}methyl)-piperidin-4-yl]-carbamate

1-[4-(trifluoromethyl)phenyl]-1H-pyrrole-3-carbaldehyde (4.054 g, 16.95 mmol) and tert-butyl piperidin-4-ylcarbamate, (3.564 g, 17.80 mmol) was suspended in DCM (35 mL). NaBH(OAc)₃ (7.184 g, 33.90 mmol) was added and stirred overnight at rt. The reaction mixture was quenched with sat. NH₄Cl aq. solution (30 mL), extracted with DCM (3×40 mL), washed with brine (30 mL), dried with Na₂SO₄ and purified with Biotage Horizon Pioneer® IIPFS using a silica cartridge and eluted with EtOAc:MeoH:TEA (gradient from 100:0:0 to 100:5:0.1) to give 6.12 g (85%) of the title compound as a solid.

¹HNMR (MeOD-d₄) δ 7.77 (d, 2H), 7.71 (d, 2H), 7.51 (s, 1H), 7.40 (t, 1H) 6.48 (m, 1H), 4.08 (s, 2H), 3.55-3.58 (m, 1H), 3.38 (d, 2H), 2.84 (t, 2H), 2.08 (m, 2H), 1.72 (m, 2H), 1.43 (s, 9H).

MS (ESI) 424.3 (M+1H⁺).

### b) 1-({1-[4-(trifluoromethyl)phenyl]-1H-pyrrol-3-yl}methyl)piperidin-4-amine dihydrochloride

tert-butyl-[1-({1-[4-(trifluoromethyl)phenyl]-1H-pyrrol-3-yl}methyl)piperidin-4-yl]carbamate (6.119 g, 14.45 mmol) was dissolved in HCl 4 M in 1,4-dioxane (35 mL) and stirred at rt. for 1.5 hours. Et₂O (10 mL) was added to the suspension which was stirred for 1.5 hours. The precipitate was filtered off and was washed with Et₂O (200 mL) and was then dried at reduced pressure over night to give 4.98 g (87%) of the title compound as a solid.

¹HNMR (MeOD-d₄) δ 7.77 (m, 4H), 7.63 (s, 1H), 7.40 (t, 1H), 6.56 (s, 1H), 4.28 (s, 2H), 3.65-3.69 (m, 2H), 3.49 (m, 1H), 3.16 (t, 2H), 2.30 (m, 2H), 1.99-2.10 (m, 2H).

MS (ESI) 325.2 (M+1H⁺).

### WORKING EXAMPLES

### Example 1

### 2,2-diphenyl-N-[1-({1-[4-(trifluoromethyl)phenyl]-1H-pyrrol-3-yl}methyl)piperidin-4-yl]acetamide

1-({1-[4-(trifluoromethyl)phenyl]-1H-pyrrol-3-yl}methyl)piperidin-4-armine dihydrochloride (0.100 g, 0.25 mmol), diphenylacetic acid (0.080 g, 0.38 mmol), potassium carbonate (0.139 g, 1.00 mmol) and EDC (0.073 g, 0.38 mmol) was dissolved in DCM:H₂O 1:1 (2 mL) and stirred at 18 hours at room temperature. The organic phase was concentrated and purified with Biotage Horizon Pioneer® HPFS using a silica cartridge with gradient elution, EtOAc and EtOAc:MeOH:TEA (100:2:0.2) to give the title compound in 0.085 g (65%).

¹H NMR (CD₃OD) δ 7.69 (d, 2H, J=9.3 Hz), 7.60 (d, 2H, J=9.3 Hz), 7.16-7.24 (m, 12H), 6.32 (m, 1H), 4.93 (s, 1H), 3.68 (m, 1H), 3.43 (s, 2H), 2.91 (d, 2H, J=11.1 Hz), 2.10 (t, 2H, J=11.7 Hz), 1.84 (d, 2H, J=11.7), 1.51 (m, 2H).

¹³C NMR (CDCl₃) δ 171.4, 143.2, 139.8, 129.1, 128.9, 127.8 (q, J=3.8 Hz), 127.4, 127.3 (q, J=33.1 Hz), 124.1 (q, J=271.8 Hz), 123.9, 119.1, 118.3, 59.4, 55.4, 52.2, 46.9, 32.2. MS ESI+) 518.4 (M+1H⁺), MS (ESI−) 516.2 (M−1H⁺).

### Example 2

### N-(3,4-difluorobenzyl)-2-[1-({1-[4-(trifluoromethyl)phenyl]-1H-pyrrol-3-yl}methyl)piperidin-4-yl]acetamide

### a) tert-butyl-4-{2-[(3,4-difluorobenzyl)amino]-2-oxoethyl}piperidine-1-carboxylate

[1-(tert-butoxycarbonyl)piperidin-4-yl]acetic acid (0.200 g, 0.82 mmol) and EDC (0.205 g, 1.0 mmol) was dissolved in DCM (6 mL). (3,4-Difluorobenzyl)amine (0.153 g, 1.0 mmol) was added the solution and stirred for 5 hours at room temperature. 10% Na₂CO₃(aq.) (4 mL) was added and the phases were separated on a phase separator, concentrated and purified with Biotage Horizon Pioneer® HPFS using a silica cartridge with gradient elution n-Heptane/EtOAc:MeOH:TEA (100:2:0.2) to give the title compound in 0.161 g (53%).

¹H NMR (CD₃OD) δ 7.30-7.12 (m, 2H), 7.21-6.98 (m, 1H), 4.32 (s, 2H), 4.04 (s, 1H), 4.01 (s, 1H), 2.74 (bs, 2H), 2.16 (d, 2H, J=6.9 Hz), 1.94 (m, 1H), 1.65 (d, 2H, J=12.1 Hz), 1.43 (s, 9H), 1.11 (m, 2H).

### b) N-(3,4-difluorobenzyl)-2-[1-({1-[4-(trifluoromethyl)phenyl]-1H-pyrrol-3-yl}methyl)piperidin-4-yl]acetamide

tert-butyl 4-{2-[(3,4-difluorobenzyl)amino]-2-oxoethyl}piperidine-1-carboxylate (0.161 g, 0.44 mmol) was dissolved in 4M HCl in 1,4-dioxane (10 mL) and stirred for 1.5 hours at room temperature. Added Et₂O (10 mL). The precipitate was filtered off, washed with Et₂O and dried. The precipitate, 1-[4-(trifluoromethyl)phenyl]-1H-pyrrole-3-carbaldehyde (0.115 g, 0.48 mmol), NaBH(OAc)₃ (0.185 g, 0.87 mmol) and DIPEA (0.056 g, 0.44 mmol) was dissolved in DCM (8 mL) and stirred for 5 hours at room temperature. Added sat. NH₄Cl(aq.) (5 mL) and separated on phase separator, concentrated and purified with Biotage Horizon Pioneer® HPFS using a silica cartridge with EtOAc:MeOH:TEA (100:2:0.2) to give the title compound in 0.125 g (58%).

¹H NMR (CDCl₃) δ 7.63 (d, 2H, J=9.2 Hz), 7.42 (d, 2H, J=9.2 Hz), 7.01-7.09 (m, 4H), 6.94 (m, 1H), 6.30 (m, 1H), 6.17 (t, 1H, J=5.8 Hz), 4.33 (d, 2H, J=6.0 Hz), 3.41 (s, 2H), 2.94 (d, 2H, J=11.0), 2.10 (d, 2H, J=7.7 Hz), 1.96 (m, 2H), 1.82 (m, 1H), 1.68 (d, 2H, J=11.6), 1.28 (m, 2H).

¹³C NMR (CDCl₃) δ 172.3, 151.4 (dd, J=13.6 Hz, J=68.2 Hz), 148.9 (dd, J=12.9 Hz, J=68.2 Hz), 143.2, 135.9 (dd, J=3.92 Hz), 127.3 (q, J=32.6 Hz), 127.1 (q, J=3.9 Hz), 124.3 (q, J=273.2 Hz), 123.7, 123.8 (dd, J=3.8 Hz), 129.6, 119.0, 118.4, 117.6 (d, J=17.6 Hz), 116.8 (d, J=17.6 Hz), 113.4, 55.7, 53.6, 43.9, 42.7, 33.5, 32.4.

MS (ESI+) 492.3 (M+1H⁺), MS (ESI−) 490.2 (M−1H⁺).

### Example 3

### N-(2-phenylethyl)-1-({1-[4-(trifluoromethyl)phenyl]-1H-pyrrol-3-yl}methyl)piperidine-4-carboxamide

### a) tert-butyl 4-{[(2-phenylethyl)amino]carbonyl}piperidine-1-carboxylate

1-(tert-butoxycarbonyl)piperidine-4-carboxylic acid (0.500 g, 2.18 mmol) and EDC (0.543 g, 2.84 mmol) was dissolved in DCM (10 mL). (2-Phenylethyl)amine (0.344 g, 2.84 mmol), DCM (20 mL) was added and the solution wvas stirred for 5 hours at room temperature. 10% Na₂CO₃(aq.)(20 mL) was added and the phases were separated on a phase separator, concentrated and purified with Biotage Horizon Pioneer® HPFS using a silica cartridge with gradient elution n-Heptane/EtOAc:MeOH:TEA (100:2:0.2) to give the title compound in 0.244 g (37%).

¹H NMR (CD₃OD) δ 7.22 (m, 5H), 4.82 (bs, 1H), 4.05 (m, 2H), 3.39 (bs 2H), 2.77 (bs, 4H), 2.29 (bs, 1H) 1.64 (bs, 2H), 1.44 (m, 10H).

### b) N-(2-phenylethyl)-1-({1-[4-(trifluoromethyl)phenyl]-1H-pyrrol-3-yl}methyl)piperidine-4-carboxamide

tert-butyl 4-{[(2-phenylethyl)amino]carbonyl}piperidine-1-carboxylate (0.244 g, 0.73 mmol) was dissolved in 4M HCl in 1,4-dioxane (10 mL) and stirred for 1.5 hours at room temperature and Et₂O 10 mL) was added. The precipitate was filtered off, washed with diethyl ether and dried. The precipitate, 1-[4-(trifluoromethyl)phenyl]-1H-pyrrole-3-carbaldehyde (0.193 g, 0.81 mmol), NaBH(OAc)₃ (0.311 g, 1.47 mmol) and DIPEA (0.095 g, 0.73 mmol) was dissolved in DCM (8 mL) and stirred for 5 hours at room temperature. Saturated NH₄Cl (aq.) (5 mL) was added and the phases were separated on a phase separator, concentrated and purified with Biotage Horizon Pioneer® HPFS using a silica cartridge with EtOAc:MeOH:TEA (100:2:0.2) to give the title compound in 0.228 g (68%).

¹H NMR (CDCl₃) δ 7.63 (d, 2H, J=8.8 Hz), 7.42 (d, 2H, J=8.8 Hz), 7.21 (m, 5H), 7.03 (m, 2H), 6.30 (s, 1H), 5.78 (m, 1H), 3.49 (q, 2H, J=6.20), 3.42 (s, 2H) 2.99 (d, 2H, J=12.4) 2.79 (t, 2H, J=6.9), 1.90-2.10 (m, 3H), 1.67-1.82 (m, 4H).

¹³H NMR (CDCl₃) δ 175.3, 143.2, 139.2, 124.3 (q, J=271 Hz), 129.0, 128.8, 127.3 (q, J=32.4 Hz), 127.1 (q, J=3.7 Hz), 126.7, 123.7, 119.6, 119.1, 118.3, 113.2, 55.6, 53.2, 43.6, 40.7, 35.9, 29.2.

### Example 4

### N-[bis(4-fluorophenyl)methyl]-2-[1-({1-[5-(trifluoromethyl)pyridin-2-yl]-1H-pyrrol-3-yl}methyl)piperidin-4-yl]acetamide

### a) [bis(4-fluorophenyl)methyl]anine

bis-(4-fluorophenyl)methanone (3.0 g, 13.7 mmol) was added to methanol (30 mL). and ammonium acetate (7.4 g, 96.2 mmol) was added. The mixture was stirred for 0.5 hours. NaBH₃CNI (0.95 g, 15.1 mmol) was added and the mixture was stirred over night. Further NaBH₃CN (0.5 g, 7.95 mmol) was added and the mixture was refluxed over night. Evaporated, added 1% Na₂CO₃ (aq) solution (40 mL), extracted with ethylacetate (3×60 mL). The organic phases was washed with brine (40 mL), dried (Na₂SO₄) and purified with Biotage Horizon Pioneer®E HPFS using a silica cartridge with gradient elution EtOAc/n-Heptane to give the title compound in 1.155 g (38%).

¹H NMR (CDCl₃) δ 7.24 (m, 4H), 7.00 (m, 4H), 6.16 (s, 2H), 5.26 (s, 1H).

### b) tert-butyl-4-(2-{[bis(4-fluorophenyl)methyl]amino}-2-oxoethyl)piperidine-1-carboxylate

[1-(tert-butoxycarbonyl)piperidin-4-yl]acetic acid (0.500 g, 2.06 mmol), [bis(4-fluorophenyl)methyl]amine (0.496 g, 2.26 mmol) and EDC (0.433 g, 2.26 mmol) was added to DCM (20 mL) and stirred for 6 hours at room temperature. The mixture was concentrated and purified with Biotage Horizon Pioneer® HPFS using a silica cartridge with gradient elution EtOAc:MeOH:TEA (100:2:0.2)/n-Heptane to give the title compound in 0.455 g (50%).

¹H NMR (CDCl₃) δ 7.13 (m, 4H), 6.95 (m, 4H), 6.8 (d, 1H), 6.18 (d, 1H), 3.96 (d, 2H), 2.61 (bs, 2H), 2.10 (m, 2H), 1.93 (m, 1H), 1.58 (d, 2H), 1.40 (s, 9H), 1.03 (m, 2H).

### c) N-[bis(4-fluorophenyl)methyl]-2-piperidin-4-ylacetamide hydrochloride

tert-butyl-4-(2-{[bis(4-fluorophenyl)methyl]amino}-2-oxoethyl)piperidine-1-carboxylate (0.455 g, 1.02 mmol) was dissolved in a 4 M HCl solution in 1,4-dioxane and was stirred for 2 hours at room temperature, whereafter Et₂O (25 mL) was added. The precipitate was filtered of and washed with Et₂O (30 mL) and dried in vacuo to give the title compound in 0.336 g (86%).

¹H NMR (CD₃OD) δ 7.25 (m, 4H), 7.06 (m, 4H), 6.18 (m, 1H), 3.35 (d, 2H), 2.97 (m, 2H), 2.30 (d, 2H), 2.08 (m, 1H), 1.90 (d, 2H), 1.45 (bq, 2H).

MS (ESI+) 345.2 (M+1H⁺), MS (ESI−) 343.1 (M-1H⁺).

### d) N-[bis(4-fluorophenyl)methyl]-2-[1-({1-[5-(trifluoromethyl)pyridin-2-yl]-1H-pyrrol-3-yl}methyl)piperidin-4-yl]acetamide

N-[bis(4-fluorophenyl)methyl]-2-piperidin-4-ylacetamide hydrochloride (0.193 g, 0.51 mmol), 1-[5-(trifluoromethyl)pyridin-2-yl]-1H-pyrrole-3-carbaldehyde (0.134 g, 0.56 mmol), NaBH(OAc)₃ (0.118 g, 0.56 mmol) and DIPEA (0.065 g, 0.51 mmol) were added to DCM (5 mL) and stirred at 18 hours at room temperature. Additional NaBH(OAc)₃, (0.100 g, 0.27 mmol) and DCM (10 mL) was added and the mixture was stirred for 6 hours. Saturated NH₄Cl (aq) (15 mL) was added. The organic phase was separated, concentrated and was the purified with Biotage Horizon Pioneer® HPFS using a silica cartridge with gradient elution EtOAc:MeOH:TEA (100:5:0.5)/n-Heptane to give the title compound in 0.175 g (60%).

¹H NMR (CDCl₃) δ 8.62 (bs, 1H), 7.90 (m, 1H), 7.47 (m, 1H), 7.41 (m, 1H), 7.32 (d, 1H), 7.13 (m, 4H), 6.97 (m, 4H), 6.32 (m, 1H), 6.28 (d, 1H), 6.17 (d, 1H), 3.40 (s, 2H), 2.92 (m, 2H), 2.12 (d, 2H), 1,94 (m, 2H), 1.80 (m, 1H), 1.65 (bd, 2H), 1.28 (m, 2H).

¹³CNMR (CDCl₃): δ 171.4, 162.1 (d, J=242), 153.4, 146.4 (q, J=4.2), 137.4 (d, J=3.1), 136.0 (q, J=3.3), 129.2 (d, J=7.6), 124.7, 123.8 (q, J=272), 122.7 (q, J=33), 118.5, 117.6, 115.8 (d, J=22), 114.3, 110.5, 55.8, 55.7, 53.6, 43.9, 33.6, 32.4 MS (ESI+) 569.3 (M+1H⁺), MS (ESI−) 567.2 (M−1H⁺).

### Example 5

### N-[4-(brifluoromethoxy)phenyl]-N′-[1-({1-[4-(trifluoromethyl)phenyl]-1H-pyrrol-3-yl}methyl)piperidin-4-yl]urea

1-({1-[4-(trifluoromethyl)phenyl]-1H-pyrrol-3-yl}methyl)piperidin-4-amine dihydrochloride (0.050 g, 0.126 mmol), 1-isocyanato-4-(trifluoromethoxy)benzene (0.038 g, 0.19 mmol) and DIPEA (0.040 g, 0.31 mmol) were dissolved in dry TIF (4 mL) and stirred for 18 hours at room temperature. The organic phase was concentrated and purified with prep. HPLC to give the title compound in 0.034 g (51%).

¹H NMR (CD₃OD) δ 7.73 (d, 2H, J=9.6 Hz), 7.65 (d, 2H, J=Hz), 7.42 (d, 2H, J=8.5), 7.33 (s, 1H), 7.30 (s, 1H), 7.12 (d, 2H, J=9.6 Hz), 6.39 (s, 1H), 3.65 (m, 3H), 3.09 (d, 2H, J=11.9), 2.45 (t, 2H, J=1.1), 2.00 (m, 2H), 1.6 (m, 2H).

MS (ESI+) 527.4 (M+1H⁺), MS (ESI−) 525.1 (M−1H⁺).

### Example 6

### N-(2,4-dichlorophenyl)-N′-[1-({1-[4-(trifluoromethyl)phenyl]-1H-pyrrol-3-yl}methyl)piperidin-4-yl]urea

1-({1-[4-(trifluoromethyl)phenyl]-1H-pyrrol-3-yl}methyl)piperidin-4-amine dihydrochloride (0.100 g, 0.25 mmol), 2,4-dichloro-1-isocyanatobenzene (0.063 g, 0.33 mmol) and DIPEA (0.075 g, 0.69 mmol) were dissolved in dry THF (4 mL) and stirred 18 hours at room temperature. The organic phase was concentrated and purified with Biotage Horizon Pioneer® HPFS using a silica cartridge with EtOAc:MeOH:TEA (100:5:0.1) to give the title compound in 0.087 g (67%).

¹H NMR (CD₃OD) δ 8.04 (d, 1H, J=9.9 Hz), 7.73 (d, 2H, J=8.6 Hz), 7.66 (d, 2H, J=8.6 Hz), 7.39 (m, 1H), 7.28 (m, 2H), 7.22 (m, 1H), 6.36 (m, 1H), 3.65 (m, 1H), 3.5 (s, 2H), 2.93 (m, 2H), 2.24 (t, 2H, J=100.5 Hz), 1.97 (m, 2H), 1.53 (m, 2H).

MS (ESI+) 512 (M+1H⁺), MS (ESI−).

### Example 7

### N-1-naphthyl-N′-[1-({1-[4-(trifluoromethyl)phenyl]-1H-pyrrol-3-yl}methyl)piperidin-4-yl]urea

1-({1-[4-(trifluoromethyl)phenyl]-1H-pyrrol-3-yl}methyl)piperidin-4-amine dihydrochloride (0.050 g, 0.13 mmol), 1-isocyanatonaphthalene (0.031 g, 0.19 mmol), and DIPEA (0.040 g, 0.31 mmol) were dissolved in dry THF (4 mL) and stirred 18 hours at room temperature. The organic phase was concentrated and purified with Biotage Horizon Pioneer®(HPFS using a silica cartridge with EtOAc:MeOH:TEA (100:5:0.1) to give the title compound in 0.030 g (46%).

¹H NMR (CD₃OD) δ 7.97 (d, 1H, J=8.7 Hz), 7.85 (d, 1H, J=8.7), 7.62-7.75 (m, 6H), 7.39-7.53 (m, 3H), 7.30 (m, 2H), 6.38 (m, 1H), 3.68 (m, 1H), 3.59 (m, 2H), 3.03 (d, 2H, J=10.8 Hz), 2.34 (t, 2H, J=10.8 Hz), 2.02 (d, 2H, J=13.1 Hz), 1.59 (m, 2H).

### Example 8

### N-(3-fluorobenzyl)-N′-[1-({1-[4-(trifluoromethyl)phenyl]-1H-pyrrol-3-yl}methyl)piperidin-4-yl]urea

1-({1-[4-(trifluoromethyl)phenyl]-1H-pyrrol-3-yl}methyl)piperidin-4-amine dihydrochloride (0.163 g, 0.4.1 mmol), 1-fluord-3 (isocyanatomethyl)benzene (0.092 g, 0.61 mmol) and DIPEA (0.130 g, 1.00 rirlnol) were dissolved in THF (5 mL) and stirred 18 hours at room temperature. The organic phase was concentrated and purified with Biotage Horizon Pioneer® HPFS using a silica cartridge with EtOAc:MeOH:TEA (100:5:0.1) to give the title compound in 0.118 g (61%).

¹H NMR (CD₃OD) δ 7.71 (d, 2H), 7.64 (d, 2H), 7.26-7.31 (m, 3H), 6.92-7.07 (m, 3H), 6.35 (m, 1H) 4.29 (s, 2H), 3.52 (m, 1H), 3.48 (s, 2H), 2.93 (d, 2H) 2.18 (t, 2H), 1.89 (m, 2H), 1.48 (m, 2H).

MS (ESI+) 475.4 (M+1H⁺), MS (ESI−) 473.1 (M −1H⁺).

### Example 9

### N,N-bis(4-fluorophenyl)-N′-[1-({1-[5-(trifluoromethyl)pyridin-2-yl]-1H-pyrrol-3-yl}methyl)piperidin-4-yl]urea

### a) N,N-bis-(4-fluoro-phenyl)-acetamide

Acetic anhydride (2.76 g, 27 mmol) was added dropwise to 4-fluoroaniline (3.0 g, 27 mmol) under an atmosphere of nitrogen. The mixture solidified during the addition. 1-Bromo-4-fluorobenzene (4.78 g, 27 mmol) was added to the mixture. Potassium carbonate (5.3 g, 38 mmol) and copper iodide (500 mg) was added and the mixture was heated to 240° C. and the mixture was stirred for 4 h. The mixture was diluted first with xylene and then, after cooling to room temperature, with DCM. The organic layers were combined and the solvent was removed. Purification on silica gel eluting with DCM:MeOH (99:1→9:1) gave 3.0 g (46% yield) of the title compound.

### b) bis-(4-fluoro-phenyl)-amine

N,N-bis-(4-fluoro-phenyl)-acetamide (2.23 g, 9.2 mmol) in MeOH (30 mL) and HCl (10% aq, 30 mL) was refluxed at 100° C. over night. LC-MS indicated presence of the title compound in the mixture. The mixture was made basic by addition of aq. NaOH (15%). The methanol was removed by evaporation and the aq. layer was extracted with CHCl₃. The organic layer was separated and dried over Na₂SO₄. The solvent was removed by evaporation to give 1.70 g (90%) of the title compound as an oil.

### c) 4-[3,3-bis-(4-fluoro-phenyl)-ureido]-piperidine-1-carboxylic acid tert-butyl ester

To a solution of triphosgene (5.1 g, 17.2 mmol) in THF (60 mL), tert-butyl 4-aminopiperidine-1-carboxylate (1.72 g, 8.58 mmol) in THF (10 mL) and triethylamine (1.73 g, 17.2 mmol) was added drop wise over 35 min. at −5° C. the mixture was stirred for 0.5 h and then refluxed for 1 h. The thick white solution was then filtered and the filtrate was concentrated to ca 10 mL. 50 mL THF was added to the solution and the evaporation procedure was repeated 3 times. The solution was then added drop wise to a solution of bis-(4-fluoro-phenyl)-amine (1.2 g, 5.84 mmol) in THF (30 mL) under an atmosphere of nitrogen. The mixture was stirred at room temperature over night and then refluxed at 90° C. for 4 h. The solvent was removed by evaporation and the residue was washed with aq. NaOH (15%) and DCM. The organic layer was separated and the solvent was removed. Purification on silica gel eluting first with Heptane:EtOAc (4:1) and then with pure MeOH gave small amount of title compound (LC-MS analysis) which was taken to the next step without any further purification.

### d) 1,1-bis-(4-fluoro-phenyl)-3-piperidin-4-yl-urea

TFA (3 mL) was added to a solution of the collected 4-[3,3-Bis-(4-fluoro-phenyl)-ureido]-piperidine-1-carboxylic acid tert-butyl ester in DCM (10 mL) and the mixture was stirred until LC-MS indicated the completion of the reaction. Aq. 2 N NaOH was added and the mixture was stirred. The organic layer was separated and the solvent was removed. Purification on silica gel eluting with DCM:MeOH (9:1<7:3 containing 0.1% ammonia (25% aq. solution)) gave 136 mg of a brown residue which was dissolved in EtOAc and washed with sat. aq. K₂CO₃. The organic layer was separated and evaporated to dryness to give 126 mg (6.5% overall yield) of the title compound.

### e) N,N-bis(4-fluorophenyl)-N′-[1-({1-[5-(trifluoromethyl)pyridin-2-yl]-1H-pyrrol-3-yl}methyl)piperidin-4-yl]urea

N,N-bis(4-fluorophenyl)-N′-piperidin-4-ylurea (126 mg, 0.38 mmol) and 1-[5-(trifluoromethyl)pyridin-2-yl]-1H-pyrrole-3-carbaldehyde (1.2 eq) were dissolved in DCM (7.5 ml) in a 16 ml vial and stirred for 10 minutes. MP-BH(OAc)₃ (2.5 eq) was added and the vial loosely sealed with a cap and stirred at rt for 2 h. The reaction was filtered washing with MeOH (2 ml) and the filtrate evaporated in vacuo to yield a yellow oil. Flash chromatography on the Biotage 9 g column using gradient EtOAc:MeOH:TEA (100:5:0.5) 10-100% over 540 mL against EtOAc gave the product as a foam (157 mg, 74%).

¹HNMR (CDCl₃) δ 8.6 (s, 1H), 7.90 (d, 1H), 7.45 (d, 2H), 7.35 (d, 1H), 7.2 (t, 4H), 7.0 (t, 4H) 6.3 (s, 1H), 4.40 (d, 1H), 3.7 (m, 1H), 3.40 (s, 2H), 2.80 (d, 2H), 2.20 (t, 2H), 1.90 (d, 2H), 1.40 (m, 2H).

¹³CNMR (CDCl₃): δ 160.9 (d, J=247), 155.6, 153.4, 146.3 (q, J=3.3), 138.8 (d, J=3.3), 136.0 (q, J=3.3), 129.1 (d, J=8.3), 123.8 (q, J=271), 123.7, 122.8 (q, J=35), 118.7, 117.8, 116.5 (d, J=22.3), 114.1, 110.5, 55.0, 51.9, 47.9, 32.3.

MS (ESI+) 556.4 (M+H⁺)

### Example 10

### N-(diphenylmethyl)-N′-[1-({1-[4-(trifluoromethyl)phenyl]-1H-pyrrol-3-yl}methyl)piperidin-4-yl]urea

### a) tert-butyl-[1-({1-[5-(trifluoromethyl)pyridin-2-yl]-1H-pyrrol-3-yl}methyl)piperidin-4-yl]carbamate

1-[5-(trifluoromethyl)pyridin-2-yl]-1H-pyrrole-3-carbaldehyde (1.500 g, 6.24 mmol), tert-butyl piperidin-4-ylcarbamate (1.313 g, 6.55 mmol) and sodium triacetoxyborohydride (2.647 g, 12.50 mmol) were dissolved in DCM (35 mL) and stirred over night at room temperature. Saturated aq. NH₄Cl was added to the reaction mixture and the organic phase was extracted with DCM (3*40 mL), dried, evaporated and purified with Biotage Horizon Pioneer® HPFS using a silica cartridge with EtOAc:MeOH:TEA (100:2:0.2) to give the title compound in 1.752 g as a solid (66%).

¹H NMR (CD₃OD) δ 8.71 (s, 1H), 8.14 (d, 1H), 7.63-7.72 (m, 3H), 6.40 (m, 1H), 3.49 (s, 2H), 3.34-3.39 (m, 1H), 2.98 (s, 1H), 2.96 (s, 1H), 2.17 (t, 2H), 1.90 (s, 1H), 1.87 (s, 1H), 1.50-1.57 (m, 2H), 1.46 (s, 9H).

### b) 1-({1-[5-(trifluoromethyl)pyridin-2-yl]-1H-pyrrol-3-yl}methyl)piperidin-4-amine trihydrochloride

tert-butyl-[1-({1-[5-(trifluoromethyl)pyridin-2-yl]-1H-pyrrol-3-yl}methyl)piperidin-4-yl]carbamate (1.752 g, 4.13 mmol) was dissolved in 4M HCl in 1,4-dioxane (50 mL and stirred for 4 hours. Diethyl ether (50 mL) was added and the resulting precipitate filtered and washed with diethyl ether (100 mL) and dried in vacuo to give the title compound in 1.576 g as a solid (88%).

¹H NMR (CD₃OD) δ 8.75 (s, 1H), 8.19-8.22 (m, 1H), 8.00 (s, 1H), 7.77-7.82 (m, 2H), 6.58 (m, 1H), 4.29 (s, 2H), 3.67 (s, 2H), 3.49 (m, 1H), 3.16 (t, 2H), 2.28 (m, 2H), 2.03 (q, 2H).

### c) N-(diphenylmethyl)-N′-[1-({1-[4-(trifluoromethyl)phenyl]-1H-pyrrol-3-yl}methyl)piperidin-4-yl]urea

1-({1-[4-(trifluoromethyl)phenyl]-1H-pyrrol-3-yl}methyl)piperidin-4-amine dihydrochloride (0.090 g, 0.23 mmol), 1,1′-(isocyanatomethylene)dibenzene (0.057 g, 0.27 mmol) and DIPEA (0.088 g, 0.68 mmol) were dissolved in THF (7 mL) and stirred at room temperature for 24 h. The organic phase was concentrated in vacuo and purified with Biotage Horizon Pioneer® HPFS using a silica cartridge with a gradient elution of EtOAc:MeOH:TEA (100:10:1) in EtOAc to give the title compound as a solid 0.112 g (93%).

¹H NMR (CD₃OD) δ 7.69 (d, 2H), δ 7.53 (d, 2H), δ 7.2-7.35 (m, 10H), δ 7.14 (m, 2H), δ 6.35 (s, 1H), δ 6.00 (s, 1H), δ 3.56 (t, 1H), δ 3.47 (s, 2H), δ 2.91 (d, 2H), δ 2.16 (t, 2H), δ 1.92 (d, 2H), δ 1.44 (q, 2H).

¹³C NMR (CD₃OD) δ 158.2, 143.2, 143.0, 128.5, 127.3, 127.1, δ 127.0 (q, J=33), δ 126.9 (q, J=4), δ 124.3 (q, J=270), δ 122.0, 119.6, 119.3, 118.9, 113.2, 57.7, 55.1, 52.1, 46.8, 32.2.

MS (ESI+) 533.2 (M+1H⁺), MS (ESI−) 591.2 (M−1H).

### Example 11

### N-[bis(4-fluorophenyl)methyl]-2-[1-({1-[5-(trifluoromethyl)pyridin-2-yl]-1H-pyrrol-3-yl}methyl)pyrrolidin-3-yl]acetamide

### a) tert-butyl 3-(2-{[bis(4-fluorophenyl)methyl]amino}-2-oxoethyl)pyrrolidine-1-carboxylate

To [1-(tert-butoxycarbonyl)pyrrolidin-3-yl]acetic acid (100 mg, 0.436 mmol) dissolved in DCM (7 ml) was added sequentially HOAt (0.436 mmol) and EDC.HCl (0.436 mmol) and the mixture was stirred for 5 h. [Bis(4-fluorophenyl)methyl]amine (105 mg, 0.480 mmol) was then added and the reaction stirred at abient temperature, for 18 hours. The solvent was reduced to about 1 ml and loaded onto 9 g biotage flash silica column and eluted with EtOAc and Heptane 20%-70% over 540 ml, to provide the title compound as an oil (146 mg, 78% yield).

MS (ESI+) 375.2 (M-^{t}Bu+H⁺), 431.2 (M+1H⁺); MS (ESI−) 429.1 (M−1H⁺).

### b) N-[bis(4-fluorophenyl)methyl]-2-[1-({1-[5-(trifluoromethyl)pyridin-2-yl]-1H-pyrrol-3-yl}methyl)pyrrolidin-3-yl]acetamide

To tert-butyl 3-(2-{[bis(4-fluorophenyl)methyl]amino}-2-oxoethyl)pyrrolidine-1-carboxylate (146 mg, 0.339 mmol) was added 4M HCl in Dioxane (10 ml) and the mixture stirred for 2 hours. The solvents were removed in vacuo, co-evaporating with dioxane (2×5 ml). The residue was taken up in DCM (7 ml) to which was added DIPEA (0.68 mmol) and 1-[5-(trifluoromethyl)pyridin-2-yl]-1H-pyrrole-3-carbaldehyde (90 mg, 0.373 mmol) and the mixture stirred for 10 minutes. MP-BH(OAc)₃ (500 mg, 3 eq) was then added and the reaction gently stirred for 3 hours. The reaction was filtered, washing with MeOH/DCM (1:1, 2 ml), and the filtrate reduced in vacuo to about 3 ml and then loaded onto a 40 g Biotage flash silica column and eluted with a gradient of EtOAc/MeOH/TEA 100:5:0.5 and heptane 10%-100% over 24×27 ml, to yeild the product as a foam (186 mg, 55%).

¹H NMR (CDCl₃) δ 8.6 (bs, 1H), 7.9 (d, 1H), 7.6 (d, 1H), 7.4 (s, 1H), 7.35 (s, 1H), 7.25 (d, 1H), 7.1 (m, 4H), 6.9 (m, 4H), 6.2 (m, 2H), 3.4 (m, 2H), 2.3-2.7 (m, 7H), 2.0 (m, 1H), 1.45 (m, 1H).

¹³CNMR (CDCl₃): δ 171.2, 162.1 (2d, J=245), 153.3, 146.4 (q, J=4.2), 137.6 (2d, J=3.1), 136.0 (q, J=3.0), 129.2 (2d, J=7.6), 125.0, 123.8 (q, J=271), 122.8 (q, J=32), 118.5, 117.2, 115.6 (2d, J=21.5), 113.8, 110.5, 59.6, 55.7, 54.1, 52.3, 42.5, 33.8, 30.2.

MS (ESI+) 555.2 (M+1H⁺); MS (ESI−) 553.1 (M−1H⁺).

### Example 12

### N-(4-fluorophenyl)-1-({1-[5-(trifluoromethyl)pyridin-2-yl]-1H-pyrrol-3-yl}methyl)piperidine-4-carboxamide acetate a) ethyl 1-({1-[5-(trifluoromethyl)pyridin-2-yl]-1H-pyrrol-3-yl}methyl)piperidine-4-carboxylate

MP-BH(OAc)₃ (2.765 g, 5.72 mmol) was added to a stirred solution of ethyl piperidine-4-carboxylate (0.300 g, 1.91 mmol) and 1-[5-(trifluoromethyl)pyridin-2-yl]-1H-pyrrole-3-carbaldehyde (0.502 g, 2.10 mmol) in DCM (20 mL) and stirred overnight. The reaction mixture was filtrated, concentrated and purified with Biotage Horizon Pioneer® HPFS using a silica cartridge with gradient elution n-Heptan/EtOAc:MeOH:TEA (100:5:0.5) to give the title compound in 0.648 g (89%).

¹H NMR (CD₃OD) δ 8.62 (m, 1H), 8.05 (m, 1H), 7.52-7.62 (m, 3H), 6.32 (m, 1H), 4.09 (q, 2H), 3.40 (s, 2H), 2.89 (d, 2H), 2.27 (m, 1H), 2.06 (t, 2H), 1.82-1.89 (m, 2H), 1.64-1.76 (m, 2H), 1.20 (t, 3H).

### b) N-(4-fluorophenyl)-1-({1-[5-(trifluoromethyl)pyridin-2-yl]-1H-pyrrol-3-yl}methyl)piperidine-4-carboxamide acetate

Lithium hydroxide (0.102 g, 4.25 mmol) was dissolved in water (5 mL) and added to a stirred solution of ethyl 1-({1-[5-(trifluoromethyl)pyridin-2-yl]-1H-pyrrol-3-yl}methyl)piperidine-4-carboxylate (0.648 g, 1.70 mmol) in tetrahydrofurane (10 mL), and stirred over night at room temperature. 4M HCl in 1,4-dioxane (10 mL) was added to the reaction mixture, and the mixture was evaporated. Acetonitrile:Water (1:1, 10 mL) was added to the reaction mixture and freeze-dried.

(4-fluorophenyl)amine (0.017 g, 0.16 mmol), HATU (0.059 g, 0.16 mmol) and DIPEA (0.091 g, 0.71 mmol) were added to the freeze-dried product (0.14 mmol) dissolved in DMF (5 mL) and stirred for 18 hours at room temperature. The organic phase was purified by preparative HPLC to give the title compound in 0.053 g (74%).

¹H NMR (CD₃OD) δ 8.70 (m, 1H), 8.14 (d, 1H), 7.79 (s, 1H), 7.68-7.74 (m, 2H), 7.50-7.55 (m, 2H), 7.01 (t, 2H), 6.45 (m, 1H), 3.90 (s, 2H), 3.36 (m, 2H), 2.60-2.69 (m, 2H), 2.48-2.57 (m, 1H), 1.96-2.02 (m, 4H), 1.93 (s, 3H).

MS (ESI+) 446.8 (M+1H⁺), MS (ESI−) 444.9 (M−1H).

### Example 13

### N-methyl-N-phenyl-N′-[1-({1-[5-(trifluoromethyl)pyridin-2-yl]-1H-pyrrol-3-yl}methyl)piperidin-4-yl]urea

1-({1-[5-(trifluoromethyl)pyridin-2-yl]-1H-pyrrol-3-yl}methyl)piperidin-4-amine dihydrochloride (0.060 g, 0.138 mmol), methyl(phenyl)carbamic chloride (0.028 g, 0.165 mmol) and DIPEA (144 ml, 0.825 mmol) were dissolved in anhydrous THF (5 ml) and stirred at room temperature for 18 h. The organic phase was then concentrated in vacuo and purified with Biotage Horizon Pioneer® HPFS using a silica cartridge with a gradient elution of EtOAc/MeOH/TEA (100:3.0.3) in EtOAc to give the title compound as an oil 0.012 g (16%).

¹H NMR (CDCl₃) δ 8.63 (s, 1H), δ 7.91 (d, 1H), δ 7.46 (t, 1H), δ 7.42-7.36 (m, 3H) δ 7.32 (d, 1H), δ 7.27 (t, 1H), δ 7.21 (dd, 2H), δ 6.30 (dd, 1H), δ 4.16 (d, 1H), δ 3.66 (m, 1H), δ 3.38 (s, 1H), δ 3.24 (s, 3H), δ 2.77 (d, 2H), δ 2.09 (t, 2H), δ 1.87 (d, 2H), δ 1.28 (q, 2H).

MS (ESI+) 458.2 (M+1H⁺).

### Example 14

### N-(1,3-benzothiazol-2-ylmethyl)-2-[1-({1-[4-(trifluoromethyl)phenyl]-1H-pyrrol-3-yl}methyl)piperidin-4-yl]acetamide

### a) Ethyl[1-({1-[4-(trifluoromethyl)phenyl]-1H-pyrrol-3-yl}methyl)piperidin-4-yl]acetate

Ethyl piperidin-4-ylacetate (1.40 g, 8.18 mmol) dissolved in DCM (10 ml) was added to a solution of 1-[4-(trifluoromethyl)phenyl]-1H-pyrrole-3-carbaldehyde (2.35 g, 9.81 mmol) and sodium triacetoxyborohydride (5.20 g, 24.53 mmol) in DCM (20 ml). The reaction was allowed to stir at room temperature for 20 h and then quenched by the addition of water (20 ml). The organic phase was separated through a phase separator, concentrated in vacuo and purified with Biotage Horizon Pioneer® HPFS using a silica cartridge with a gradient elution of EtOAc/MeOH/TEA (100:10:1) in EtOAc to give the title compound as a solid 2.39 g (74%).

¹H NMR (CDCl₃) δ 7.65 (d, 2H), δ 7.44 (d, 2H), 7.0-7.8 (m, 2H). δ 6.30 (dd, 1H), δ 4.09 (q, 2H), δ 3.54 (s, 2H), δ 3.05 (d, 2H), δ 2.21 (d, 2H), δ 2.09 (t, 21), δ 1.85-1.66 (m, 3H), δ 1.39 (dq, 2H), δ 1.22 (t, 2H).

### b) [1-({1-[4-(trifluoromethyl)phenyl]-1H-pyrrol-3-yl}methyl)piperidin-4-yl]acetic acid-chlorolithium (1:2) hydrochloride

Lithium hydroxide (0.29 g, 12.12 mmol) dissolved in water (7 ml) was added to a solution of ethyl[1-({1-[4-(trifluoromethyl)phenyl]-1H-pyrrol-3-yl}methyl)piperidin-4-yl]acetate (2.39 g, 6.06 mmol) in THF (25 ml). The mixture was stirred at room temperature for 16 h and the solvents evaporated in vacuo. The residue was dissolved in 4 M HCl in dioxane (25 ml) and stirred at room temperature for 1 h before it was freeze-dried, yielding 3.208 g of a solid. The reaction was assumed to have 100% conversion.

¹H NMR (CD₃OD) δ 7.76 (d, 2H), δ 7.71 (d, 2H), δ 7.57 (s, 1H), δ 7.39 (t, 1H), δ 6.50 (dd, 1H), δ 4.21 (s, 2H), δ 3.55 (d, 2H), δ 2.98 (t, 2H), δ 2.28 (d, 2H), δ 2.01 (d, 3H), δ 1.54 (m, 2H).

MS (ESI+) 367.1 (M+1H⁺), MS (ESI−) 365.1 (M−1H⁺).

### c) N-(1,3-benzothiazol-2-ylmethyl)-2-[1-({1-[4-(trifluoromethyl)phenyl]-1H-pyrrol-3-yl}methyl)piperidin-4-yl]acetamide

4 ml of a stock solution containing [1-({1-[4-(trifluoromethyl)phenyl]-1H-pyrrol-3-yl}methyl)piperidin-4-yl]acetic acid-chlorolithium (1:2) hydrochloride (1.33 g, 2.73 mmol), DIPEA (1.41 g, 10.9 mmol) and HATU (1.24 g, 3.27 mmol) in DMF (100 ml) was added to a reaction vial containing 1-(1,3-benzothiazol-2-yl)methanamine hydrochloride (0.026 g, 0.13 mmol). The reaction was allowed to stir at room temperature for 16 h and was then evaporated in a vacuum centrifuge at 40° C. for 5 h. The remaining oil was dissolved in DCM (3 ml) and shaken with 1% NaHCO₃ (4 ml). The organic phase was separated through a phase separator and evaporated in a vacuum centrifuge at 20° C. for 3 h. The remaining oil was purified by Automated Preparative HPLC to give the title product 0.030 g (54%).

¹H NMR ((CD₃)₂SO) δ 8.84 (t, 1H), δ 8.05 (d, 1H), δ 7.92 (d, 1H), δ 7.79 (m, 4H), δ 7.52-7.37 (m, 4H), δ 6.28 (s, 1H), δ 4.65 (d, 2H), δ 3.50-3.35 (m, 2H), δ 2.94 (s, 2H), δ 2.13 8d, 2H), δ 2.15-1.89 (s, 2H), 61.82-1.64 (m, 1H), 61.69 (d, 2H), 61.32-1.16 (m, 2H).

MS (ESI+) 513.1 (M+1H⁺), MS (ESI−) 511.2 (M−1H).

### Example 15-48

Using the method described for the preparation of the compound of Example 14, the compounds of Example 15-48 were prepared by reaction of [1-({1-[4-(trifluoromethyl)phenyl]-1H-pyrrol-3-yl}methyl)piperidin-4-yl]acetic acid-chlorolithium (1:2) hydrochloride with commercially available amines. The isolated yields of the products were in the range 17-75% with purity in excess of 95% (assessed by HPLC-UV and ¹H NMR)

| | | MS (ESI+) |
|---|---|---|
| Example | Compound Name | (M + 1H+) |
| 15 | N-(2-furylmethyl)-2-[1-({1-[4-(trifluoromethyl)phenyl]-1H-pyrrol-3- | 446.1 |
| | yl}methyl)piperidin-4-yl]acetamide | |
| 16 | N-(2-pyridin-2-ylethyl)-2-[1-({1-[4-(trifluoromethyl)phenyl]-1H-pyrrol-3- | 471.1 |
| | yl}methyl)piperidin-4-yl]acetamide | |
| 17 | N-(2,4-dichlorobenzyl)-2-[1-({1-[4-(trifluoromethyl)phenyl]-1H-pyrrol-3- | 524 |
| | yl}methyl)piperidin-4-yl]acetamide | |
| 18 | N-(1,2-diphenylethyl)-2-[1-({1-[4-(trifluoromethyl)phenyl]-1H-pyrrol-3- | 546.2 |
| | yl}methyl)piperidin-4-yl]acetamide | |
| 19 | N-(1,3-benzodioxol-5-ylmethyl)-2-[1-({1-[4-(trifluoromethyl)phenyl]-1H- | 500.1 |
| | pyrrol-3-yl}methyl)piperidin-4-yl]acetamide | |
| 20 | N-ethyl-N-(2-pyridin-2-ylethyl)-2-[1-({1-[4-(trifluoromethyl)phenyl]-1H- | 499.2 |
| | pyrrol-3-yl}methyl)piperidin-4-yl]acetamide | |
| 21 | N-(2,3-dihydro-1,4-benzodioxin-2-ylmethyl)-2-[1-({1-[4- | 514.1 |
| | (trifluoromethyl)phenyl]-1H-pyrrol-3-yl}methyl)piperidin-4-yl]acetamide | |
| 22 | N-[3-(1H-imidazol-1-yl)propyl]-2-[1-({1-[4-(trifluoromethyl)phenyl]-1H- | 474.1 |
| | pyrrol-3-yl}methyl)piperidin-4-yl]acetamide | |
| 23 | N-(2,4-dichlorobenzyl)-2-[1-({1-[4-(trifluoromethyl)phenyl]-1H-pyrrol-3- | 470.1 |
| | yl}methyl)piperidin-4-yl]acetamide | |
| 24 | N-(4-fluorophenyl)-2-[1-({1-[4-(trifluoromethyl)phenyl]-1H-pyrrol-3- | 460.1 |
| | yl}methyl)piperidin-4-yl]acetamide | |
| 25 | N-[phenyl(pyridin-2-yl)methyl]-2-[1-({1-[4-(trifluoromethyl)phenyl]-1H- | 533.2 |
| | pyrrol-3-yl}methyl)piperidin-4-yl]acetamide | |
| 26 | N-[3-(difluoromethoxy)benzyl]-2-[1-({1-[4-(trifluoromethyl)phenyl]-1H- | 522.1 |
| | pyrrol-3-yl}methyl)piperidin-4-yl]acetamide | |
| 27 | 1-(3-methoxyphenyl)-4-{[1-({1-[4-(trifluoromethyl)phenyl]-1H-pyrrol-3- | 541.2 |
| | yl}methyl)piperidin-4-yl]acetyl}piperazine | |
| 28 | 1′-{[1-({1-[4-(trifluoromethyl)phenyl]-1H-pyrrol-3-yl}methyl)piperidin-4- | 534.2 |
| | yl]acetyl}spiro[indene-1,4′-piperidine] | |
| 29 | N-(3,3-diphenylpropyl)-2-[1-({1-[4-(trifluoromethyl)phenyl]-1H-pyrrol-3- | 560.2 |
| | yl}methyl)piperidin-4-yl]acetamide | |
| 30 | N-(1-phenylpropyl)-2-[1-({1-[4-(trifluoromethyl)phenyl]-1H-pyrrol-3- | 484.1 |
| | yl}methyl)piperidin-4-yl]acetamide | |
| 31 | N-(4-fluorophenyl)-N-methyl-2-[1-({1-[4-(trifluoromethyl)phenyl]-1H- | 474 |
| | pyrrol-3-yl}methyl)piperidin-4-yl]acetamide | |
| 32 | N-[(1R,2S)-2-phenylcyclopropyl]-2-[1-({1-[4-(trifluoromethyl)phenyl]- | 482.1 |
| | 1H-pyrrol-3-yl}methyl)piperidin-4-yl]acetamide | |
| 33 | N-(3-methylbutyl)-2-[1-({1-[4-(trifluoromethyl)phenyl]-1H-pyrrol-3- | 436 |
| | yl}methyl)piperidin-4-yl]acetamide | |
| 34 | N-[2-(3,4-dimethoxyphenyl)ethyl]-N-methyl-2-[1-({1-[4- | 544.2 |
| | (trifluoromethyl)phenyl]-1H-pyrrol-3-yl}methyl)piperidin-4-yl]acetamide | |
| 35 | N-[2-(3,4-dimethoxyphenyl)ethyl]-2-[1-({1-[4-(trifluoromethyl)phenyl]- | 530.2 |
| | 1H-pyrrol-3-yl}methyl)piperidin-4-yl]acetamide | |
| 36 | N-[4-(1,2,3-thiadiazol-4-yl)benzyl]-2-[1-({1-[4-(trifluoromethyl)phenyl]- | 540.1 |
| | 1H-pyrrol-3-yl}methyl)piperidin-4-yl]acetamide | |
| 37 | N-[1-(2,3-dihydro-1,4-benzodioxin-5-yl)ethyl]-2-[1-({1-[4- | 528.1 |
| | (trifluoromethyl)phenyl]-1H-pyrrol-3-yl}methyl)piperidin-4-yl]acetamide | |
| 38 | N,N-diethyl-1-{[1-({1-[4-(trifluoromethyl)phenyl]-1H-pyrrol-3- | 533.3 |
| | yl}methyl)piperidin-4-yl]acetyl}piperidine-3-carboxamide | |
| 39 | N-1-adamantyl-2-[1-({1-[4-(trifluoromethyl)phenyl]-1H-pyrrol-3- | 500.2 |
| | yl}methyl)piperidin-4-yl]acetamide | |
| 40 | N-[2-(4-methoxyphenoxy)ethyl]-2-[1-({1-[4-(trifluoromethyl)phenyl]-1H- | 516.1 |
| | pyrrol-3-yl}methyl)piperidin-4-yl]acetamide | |
| 41 | N-{(1S)-1-[(benzyloxy)methyl]propyl}-2-[1-({1-[4- | 528.2 |
| | (trifluoromethyl)phenyl]-1H-pyrrol-3-yl}methyl)piperidin-4-yl]acetamide | |
| 42 | N-[(1R)-1-(3-methoxyphenyl)ethyl]-2-[1-({1-[4-(trifluoromethyl)phenyl]- | 500.1 |
| | 1H-pyrrol-3-yl}methyl)piperidin-4-yl]acetamide | |
| 43 | N-{[3-(4-methoxyphenyl)isoxazol-5-yl]methyl}-2-[1-({1-[4- | 553.1 |
| | (trifluoromethyl)phenyl]-1H-pyrrol-3-yl}methyl)piperidin-4-yl]acetamide | |
| 44 | 4-(4-chlorophenyl)-1-{[1-({1-[4-(trifluoromethyl)phenyl]-1H-pyrrol-3- | 542.1 |
| | yl}methyl)piperidin-4-yl]acetyl}-1,2,3,6-tetrahydropyridine | |
| 45 | N-[(1S,2S)-2-(benzyloxy)cyclopentyl]-2-[1-({1-[4- | 540.2 |
| | (trifluoromethyl)phenyl]-1H-pyrrol-3-yl}methyl)piperidin-4-yl]acetamide | |
| 46 | N-(1-methyl-1-phenylethyl)-2-[1-({1-[4-(trifluoromethyl)phenyl]-1H- | 484.2 |
| | pyrrol-3-yl}methyl)piperidin-4-yl]acetamide | |
| 47 | N-[(1-methyl-1H-pyrrol-2-yl)methyl]-2-[1-({1-[4- | 459.1 |
| | (trifluoromethyl)phenyl]-1H-pyrrol-3-yl}methyl)piperidin-4-yl]acetamide | |
| 48 | 4-(2-oxo-2-pyrrolidin-1-ylethyl)-1-({1-[4-(trifluoromethyl)phenyl]-1H- | 420.1 |
| | pyrrol-3-yl}methyl)piperidine | |

### Example 49

### N-(2-pyridin-2-ylethyl)-1-({1-[4-(trifluoromethyl)phenyl]-1H-pyrrol-3-yl}methyl)piperidine-4-carboxamide

### a) Ethyl 1-({1-[4-(trifluoromethyl)phenyl]-1-pyrrol-3-yl}methyl)piperidine-4-carboxylate

Ethyl piperidine-4-carboxylate (1.35 g, 8.59 mmol) dissolved in DCM (5 ml) was added to a solution of 1-[4-(trifluoromethyl)phenyl]-1H-pyrrole-3-carbaldehyde (2.56 g, 10.69 mmol) and sodium triacetoxyborohydride (5.46 g, 25.8 mmol) in DCM (20 ml). The reaction was allowed to stir at room temperature for 6 h and was then quenched by addition of water (20 ml). The organic layer was separated through a phase separator, concentrated in vacuo and purified with Biotage Horizon Pioneer® HPFS using a silica cartridge with a gradient elution of EtOAc/MeOH/TEA (100:10:2) in EtOAc to give the title compound as is a solid 2.64 g (81%).

¹H NMR (CDCl₃) δ 7.65 (d, 2H), δ 7.44 (d, 2H), δ 7.08-7.02 (m, 2H), δ 6.31 (dd, 1H), δ 4.11 (q, 2H), δ 3.56 (m, 2H), δ 2.99 (d, 2H), δ 2.36-2.14 (m, 3H), 1.98-1.77 (m, 4H), δ 1.22 (t, 3H).

### b) 1-([{1-[4-(trifluoromethyl)phenyl]-1H-pyrrol-3-yl}methyl)piperidine-4-carboxylic acid-chlorolithium (1:2) hydrochloride

Lithium hydroxide (0.33 g, 13.90 mmol) dissolved in water (7 ml) was added to a solution of ethyl 1-({1-[4-(trifluoromethyl)phenyl]-1H-pyrrol-3-yl}methyl)piperidine-4-carboxylate (2.64 g, 6.95 mmol) in THF (20 ml). The mixture was allowed to stir at room temperature for 3 days and was then evaporated in vacuo. 4 M HCl in dioxane (25 ml) was added to the remaining oil, stirred at room temperature for 1 h, concentrated and freeze-dried, yielding the title compound as a solid 0.90 g (27%).

1H NMR (CD₃OD) δ 7.77-7.61 (m, 5H), δ 7.37 (s, 1H), δ 6.60 (s, 1H), δ 4.27 (s, 2H), δ 3.55 (d, 2H), δ 3.16 (t, 2H), δ 2.66 (m, 1H), δ 2.27-1.95 (m, 4H).

### c) N-(2-pyridin-2-ylethyl)-1-({1-[4-(trifluoromethyl)phenyl]-1H-pyrrol-3-yl}methyl)piperidine-4-carboxamide

4 ml of a stock solution containing 1-({1-[4-(trifluoromethyl)phenyl]-1H-pyrrol-3-yl}methyl)piperidine-4-carboxylic acid-chlorolithium (1:2) hydrochloride (0.90 g, 1.90 mmol), DIPEA (0.98 g, 7.60 mmol) and HATU (0.87 g, 2.28 mmol) in DMF (100 ml) was added to a reaction vial containing 2-pyridin-2-ylethanamine (0.011 g, 0.09 mmol). The reaction was allowed to stir at room temperature for 16 h and was then evaporated in a vacuum centrifuge at 50° C. for 4.5 h. The remaining oil was dissolved in DCM (4 ml) and shaken with 1% NaHCO₃ (aq) (4 ml). The organic layer was separated through a phase separator, evaporated in a vacuum centrifuge at 20° C. for 3 h and purified by Automated Preparative HPLC to give the title product, 0.019 g (55%).

¹H NMR ((CD₃)₂SO) δ 8.48 (d, 1H), δ 7.78 (m, 5H), 7.68 (dt, 1H), 7.45 (t, 1H), 7.38 (s, 1H), 7.23-7.17 (m, 2H), 6.24 (t, 1H), 3.37 (q, 3H), 3.32 (s, 2H), 2.80-2.91 (m, 4H), 2.01 (m, 1H), 1.85 (dt, 2H), 1.61-1.50 (m, 3H).

MS (ESI+) 457.0 (M+1H⁺), MS (ESI−) 515.1 (M−1H⁺).

### Example 50-67

Using the method described for the preparation of the compound of Example 49, the compounds of Example 50-67 were prepared by reaction of 1-({1-[4-(trifluoromethyl)phenyl]-1H-pyrrol-3-yl}methyl)piperidine-4-carboxylic acid-chloro-lithium (1:2) hydrochloride with commercially available amines. The isolated yields were in the range 27-55% with purity in excess of 94-100% (assessed by HPLC-UV and ¹H NMR)

| | | MS (ESI+) |
|---|---|---|
| Example | Compound Name | (M + 1H+) |
| 50 | N-(2,4-dichlorobenzyl)-1-({1-[4-(trifluoromethyl)phenyl]-1H-pyrrol-3- | 511.4 |
| | yl}methyl)piperidine-4-carboxamide | |
| 51 | N-(1,2-diphenylethyl)-1-({1-[4-(trifluoromethyl)phenyl]-1H-pyrrol-3- | 532.1 |
| | yl}methyl)piperidine-4-carboxamide | |
| 52 | N-(1,3-benzodioxol-5-ylmethyl)-1-({1-[4-(trifluoromethyl)phenyl]-1H- | 486 |
| | pyrrol-3-yl}methyl)piperidine-4-carboxamide | |
| 53 | N-[2-(3,4-dimethoxyphenyl)ethyl]-1-({1-[4-(trifluoromethyl)phenyl]-1H- | 516.1 |
| | pyrrol-3-yl}methyl)piperidine-4-carboxamide | |
| 54 | N-[4-(1,2,3-thiadiazol-4-yl)benzyl]-1-({1-[4-(trifluoromethyl)phenyl]-1H- | 526 |
| | pyrrol-3-yl}methyl)piperidine-4-carboxamide | |
| 55 | N-(2,3-dihydro-1,4-benzodioxin-2-ylmethyl)-1-({1-[4-(trifluoromethyl)phenyl]- | 500 |
| | 1H-pyrrol-3-yl}methyl)piperidine-4-carboxamide | |
| 56 | N-[1-(2,3-dihydro-1,4-benzodioxin-5-yl)ethyl]-1-({1-[4-(trifluoromethyl)phenyl]- | 514.1 |
| | 1H-pyrrol-3-yl}methyl)piperidine-4-carboxamide | |
| 57 | N-[phenyl(pyridin-2-yl)methyl]-1-({1-[4-(trifluoromethyl)phenyl]-1H- | 519.1 |
| | pyrrol-3-yl}methyl)piperidine-4-carboxamide | |
| 58 | N-[3-(difluoromethoxy)benzyl]-1-({1-[4-(trifluoromethyl)phenyl]-1H- | 508 |
| | pyrrol-3-yl}methyl)piperidine-4-carboxamide | |
| 59 | N-[2-(4-methoxyphenoxy)ethyl]-1-({1-[4-(trifluoromethyl)phenyl]-1H- | 502 |
| | pyrrol-3-yl}methyl)piperidine-4-carboxamide | |
| 60 | N-{(1S)-1-[(benzyloxy)methyl]propyl}-1-({1-[4-(trifluoromethyl)phenyl]- | 514.1 |
| | 1H-pyrrol-3-yl}methyl)piperidine-4-carboxamide | |
| 61 | N-{[3-(4-methoxyphenyl)isoxazol-5-yl]methyl}-1-({1-[4- | 539.1 |
| | (trifluoromethyl)phenyl]-1H-pyrrol-3-yl}methyl)piperidine-4-carboxamide | |
| 62 | 1-(3-methoxyphenyl)-4-{[1-({1-[4-(trifluoromethyl)phenyl]-1H-pyrrol-3- | 527.1 |
| | yl}methyl)piperidin-4-yl]carbonyl}piperazine | |
| 63 | 4-(4-chlorophenyl)-1-{[1-({1-[4-(trifluoromethyl)phenyl]-1H-pyrrol-3- | 528.1 |
| | yl}methyl)piperidin-4-yl]carbonyl}-1,2,3,6-tetrahydropyridine | |
| 64 | N-[(1S,2S)-2-(benzyloxy)cyclopentyl]-1-({1-[4-(trifluoromethyl)phenyl]- | 526.1 |
| | 1H-pyrrol-3-yl}methyl)piperidine-4-carboxamide | |
| 65 | N-(3,3-diphenylpropyl)-1-({1-[4-(trifluoromethyl)phenyl]-1H-pyrrol-3- | 546.1 |
| | yl}methyl)piperidine-4-carboxamide | |
| 66 | N-(1-phenylpropyl)-1-({1-[4-(trifluoromethyl)phenyl]-1H-pyrrol-3- | 470.1 |
| | yl}methyl)piperidine-4-carboxamide | |
| 67 | N-(1,3-benzothiazol-2-ylmethyl)-1-({1-[4-(trifluoromethyl)phenyl]-1H- | 499 |
| | pyrrol-3-yl}methyl)piperidine-4-carboxamide | |

### Example 68

### N-[(5-chloro-6-methoxypyridin-3-yl)(4-fluorophenyl)methyl]-2-[1-({1-[4-(trifluoromethyl)phenyl]-1H-pyrrol-3-yl}methyl)piperidin-4-yl]acetamide

### a) 5-chloro-N,6-dimethoxy-N-methylnicotinamide

5-chloro-6-methoxynicotinic acid (0.900 g, 4.80 mmol) was dissolved in thionyl chloride (8 mL) and the solution was refluxed at 85° C. for 4 hours. The reaction was concentrated in vacuo, redissolved in DCM (6 mL) and N,O-dimethylhydroxylamine hydrochloride (0.562 g, 5.76 mmol) was added. The stirred mixture was cooled (0° C.) and TEA (2 mL) in DCM (2 mL) was added. The reaction mixture was allowed to warm to room temperature, was stirred for 18 h, was diluted with DCM (30 mL) and was washed with sat. NaHCO₃(aq.)/H₂O (9:1, 2X²⁰ mL). The combined organic phase was dried over a phase separator and was evaporated in vacuo to give the title compound (1.0 g, 90%).

¹H NMR (CDCl₃) δ 8.52 (s, 1H), 8.06 (s, 1H), 4.05 (s, 3H), 3.56 (s, 3H), 3.48 (s, 3H).

MS (ESI+) 231.1 (M+1H⁺).

### b) (5-chloro-6-methoxypyridin-3-yl)(4-fluorophenyl)methanone

5-chloro-N,6-dimethoxy-N-methylnicotinamide (0.600 g, 2.60 mmol), dissolved in dry THF (4 mL), was added dropwise (30 min) to a stirred and cooled (−78° C.) solution of n-BuLi (1.6 M in hexane, 0.333 g) and 1-bromo-4-fluorobenzene (0.910 g, 5.20 mmol) in dry THF (10 mL). The reaction mixture was stirred at −78° C. for 2 hours followed by 1 hour at 0° C. THF (10 mL) was added to the reaction mixture and the mixture was washed with 3M HCl (aq.) (10 mL). The water phase was extracted with diethyl ether. The THF solution was washed with sat. NaHCO₃ (aq.)/H₂O (9:1, 10 mL) and the combined organic phases was dried with MgSO₄, filtered and purified by preparative HPLC to give the title compound (0.230 g, 33%).

¹H NMR (CDCl₃) δ 8.38 (s, 1H), 8.04 (s, 1H), 7.76-7.72 (m, 2H), 7.13-7.09 (m, 2H), 4.025 (s, 3H).

### c) (5-chloro-6-methoxypyridin-3-yl)(4-fluorophenyl)methanone oxime

(5-chloro-6-methoxypyridin-3-yl)(4-fluorophenyl)methanone (0.225 g, 0.847 mmol) and hydroxylamine hydrochloride (1.150 g, 16.55 mmol) was dissolved in EtOH (99.5%, 10 mL) and heated with microwave at 120° C. for 5 minutes. Concentrated in vacuo and to which was added sat. NaHCO₃ (aq.)./H₂O (9:1, 20 mL) and extracted with DCM, the combined organic phases were dried over MgSO₄, filtered and concentrated in vacuo to give the title compound 0.244 g (103%) as an oil.

¹H NMR (CDCl₃) (mixture of E and Z isomers) δ 9.80 (bs, 1H), 8.11 (s, ½H), 7.99 (s, ½H), 7.79 (s, 1H), 7.38 (m, 2H), 7.10 (m, 1H), 7.00 (m, 1H), 4.03 (s, 1½H), 3.99 (m, 1½H).

MS (ESI+) 281.1 (M+1H⁺), MS (ESI−) 278.9 (M−1H⁺).

### d) [(5-chloro-6-methoxypyridin-3-yl)(4-fluorophenyl)methyl]amine

A mixture of (5-chloro-6-methoxypyridin-3-yl)(4-fluorophenyl)methanone oxime (0.244 g, 0.87 mmol) and ammonium acetate (0.114 g, 1.48 mmol) in ethanol (3 mL), water (2 mL) and NH₃ (26% aq, 2.5 mL) was heated to 80° C. Zn powder (0.256 g, 3.91 mmol) was added portionwise to the reaction mixture over 1 hour. After 5 hours of stirring Zn powder (0.256 g, 3.91 mmol) and ammonium acetate (0.114 g, 1.48 mmol) were added to the reaction mixture and stirred for additional 18 hours at 80° C. Sat. NaHCO₃ (aq.)/H₂O (1:1, mL) was added to the reaction mixture and extracted with DCM, dried over MgSO₄ and concentrated in vacuo to give the title compound (0.187 g, 81%).

¹H NMR (CDCl₃) δ 7.99 (s, 1H), 7.62 (s, 1H), 7.29 (m, 2H), 6.97 (m, 2H), 5.12 (s, 1H), 3.94 (s, 3H), 1.78 (bs, 2H).

### e) tert-butyl 4-(2-{[(5-chloro-6-methoxypyridin-3-yl)(4-fluorophenyl)methyl]amino}-2-oxoethyl)piperidine-1-carboxylate

[(5-chloro-6-methoxypyridin-3-yl)(4-fluorophenyl)methyl]amine (0.187 g, 0.701 mmol) was added dropwise to a stirred solution of [1-(tert-butoxycarbonyl)piperidin-4-yl]acetic acid (0.243 g, 0.84 mmol), EDC (0.161 g, 0.84 mmol) and HOAt (0.115 g, 0.84 mmol) in DCM (5 mL), and stirred for 5 hours at room temperature. Sat. NaHCO₃ (aq.)/H₂O (9:1, mL) was added and extracted with DCM, dried over MgSO₄, filtered and concentrated in vacuo and purified with Biotage Horizon Pioneer® HPFC using a silica cartridge with elution EtOAc/n-Heptane (45:55) to give the title compound (0.307 g, 89%).

MS (ESI+) 492.1 (M+1H⁺), MS (ESI−) 490.0 (M−1H⁺).

### f) N-[(5-chloro-6-methoxypyridin-3-yl)(4-fluorophenyl)methyl]-2-[{1-[4-(trifluoromethyl)phenyl]-1H-pyrrol-3-yl}methyl)piperidin-4-yl]acetamide

tert-butyl 4-(2-{[(5-chloro-6-methoxypyridin-3-yl)(4-fluorophenyl)methyl]amino}-2-oxoethyl)piperidine-1-carboxylate (0.154 g, 0.31 mmol) was dissolved in 4 M HCl (aq) in dioxane (10 mL) and stirred for 1 hour, concentrated in vacuo and redissolved in DCM (10 mL) and DIPEA (0.121 g, 0.94 mmol) followed by addition of 1-[4-(trifluoromethyl)phenyl]-1H-pyrrole-3-carbaldehyde (0.090 g, 0.38 mmol) and NIP-triacetoxyborohydride (0.544 g, 1.13 mmol of H⁻). The mixture was stirred over night at room temperature, filtered, concentrated in vacuo and purified on preparative HPLC to give the title compound 0.110 g (57%) as a solid.

¹H NMR (CDCl₃) δ 7.90 (s, 1H), 7.63 (d, 2H, J=8.5 Hz), 7.43 (m, 3H), 7.14 (m, 2H), 6.97-7.05 (m, 4H), 6.29 (s, 1H), 6.14 (d, 1H, J=8.47 Hz), 6.00 (d, 1H, J=8.47 Hz), 3.97 (s, 3H), 3.41 (s, 2H), 2.93 (d, 2H, J=10.6 Hz), 2.13 (d, 2H, J=7.2 Hz), 1.95 (t, 2H, J=10.6 Hz), 1.81 (m, 1H), 1.66 (m, 2H), 1.29 (m, 2H).

MS (ESI+) 615.2 (M+1H⁺)

### Example 69

### N-[(5-chloro-6-oxo-1,6-dihydropyridin-3-yl)(4-fluorophenyl)methyl]-2-[1-({1-[4-(trifluoromethyl)phenyl]-1H-pyrrol-3-yl}methyl)piperidin-4-yl]acetamide acetate salt

N-[(5-chloro-6-methoxypyridin-3-yl)(4-fluorophenyl)methyl]-2-[1-({1-[4-(trifluoromethyl)phenyl]-1H-pyrrol-3-yl}methyl)piperidin-4-yl]acetamide (0.100 g, 0.163 mmol, from Example 68) and pyridine hydrochloride (0.225 g, 1.95 mmol) was heated at 145° C. for 5 minutes. The reaction mixture was allowed to cool to room temperature, then dissolved in H₂O/acetonitrile (1:1) and purified on prep-HPLC to give the title compound in 0.039 g (36%) as the acetate salt.

¹H NMR (CD₃OD) δ 7.75 (d, 2H, J=8.5 Hz), 7.68 (d, 2H, J=8.5 Hz), 7.60 (m, 1H), 7.44 (s, 1H), 7.34 (m, 1H), 7.28 (m, 2H), 7.15 (m, 1H), 7.09 (t, 2H, J=8.5 Hz), 6.44 (bs, 1H), 5.98 (s, 1H), 3.99 (s, 2H), 3.36 (d, 2H, J=11.4 Hz), 2.70 (t, 2H, J=12.5 Hz), 2.25 (d, 2H, J=7.2 Hz), 1.97 (m, 1H), 1.90 (s, 4H, AcOH), 1.84 (m, 2H), 1.54-1.42 (m, 2H).

MS (ESI+) 601.2 (M+1H⁺)

### Example 70

### N-(4-chloro-2-methoxybenzyl)-({1-[4-(trifluoremethyl)phenyl]-1H-pyrrol-3-yl}methyl)piperidine-4-carboxamide

### a) (4-chloro-2-methoxyphenyl)methanol

4-chloro-2-methoxybenzoic acid (2.00 g, 10.72 mmol) and TEA (1.94 ml, 13.9 mmol) were dissolved in THF (25 ml) and cooled to −20° C. Isobutyl chloridocarbonate (1.90 g, 13.9 mmol) was added and the reaction was stirred for 2 h during which time a white precipitate was formed. The precipitate was filtered off, washed with THF and the flask was again cooled to −20° C. Sodium borohydride (1.22 g, 32.2 mmol) was added along with a few drops of water resulting in vigorous gas evolution. The rest of the water (14 ml) was added when the gas evolution had decreased. The cooling bath was removed and the reaction was stirred for 16 h. Conc. HCl was added dropwise until the gas formation had ceased. THF was then evaporated in vacuo, the aqueous solution basified to pH 10 with NaHCO₃ (s), diluted with water and extracted twice with DCM. The combined organics were dried through a phase separator and concentrated in vacuo. Purification was done with Biotage Horizon Pioneer® HPFC using a silica cartridge with a gradient elution of 5-40% EtOAc in heptane yielding the title compound as a solid 1.16 g (63%).

¹H NMR (CDCl₃) δ 7.17 (d, 1H), 6.89 (d, 1H), 6.82 (s, 1H), 4.57 (s, 2H), 3.79 (s, 3H), 2.80 (s, OH).

### b) 4-chloro-1-(chloromethyl)-2-methoxybenzene

(4-chloro-2-methoxyphenyl)methanol (1.16 g, 6.72 mmol) and TEA (1.87 ml, 13.4 mmol) were dissolved in DCM and in an icebath under an N₂ atmosphere. Methanesulfonyl chloride (679 μl, 8.74 mmol) was added over a period of 30 min and the reaction stirred at 0° C. for 2 h. DCM and 1 M HCl (aq.) were added, the phases separated and the aqueous phase extracted with DCM. The combined organics were dried through a phase separator and evaporated in vacuo. Purification was done with Biotage Horizon Pioneer® HPFS using a silica cartridge with a gradient elution of 0-30% EtOAc in heptane yielding the title compound as a white solid 622 mg (48%).

¹H NMR (CDCl₃) δ 7.25 (d, 1H), 6.91 (dd, 1H), 6.86 (d, 1H), 4.54 (s, 2H), 3.83 (s, 3H).

¹³C NMR (CDCl₃) δ 158.1, 135.6, 131.5, 124.7, 120.9, 111.8, 56.0, 41.1.

### c) 1-(azidomethyl)-4-chloro-2-methoxybenzene

4-chloro-1-(chloromethyl)-2-methoxybenzene (622 mg, 3.26 mmol) was dissolved in DMF and NaN₃ (423 mg, 6.51 mmol) was added followed by a few drops of water. The reaction was stirred at room temperature for 16 h. It was then poured on to water and extracted with ether (3×). The combined organic layers were washed with water dried through a phase separator and evaporated in vacuo and was used in the following step without further purification.

¹H NMR (CDCl₃) δ7.15 (d, 1H), 6.92 (dd, 1H), 6.88 (ds, 1H), 4.29 (s, 2H), 3.83 (s, 3H).

### d) 1-(4-chloro-2-methoxyphenyl)methanamine

1-(azidomethyl)-4-chloro-2-methoxybenzene (560 mg, 2.83 mmol) was dissolved in THF. (10 ml) to which was added subsequently triphenylphosphine (1.04 g, 3.97 mmol) and water (143 μl, 7.93 mmol) and the reaction was then stirred at rt for 3 days. The reaction mixture was then poured over 1 M HCl and separated with EtOAc. The organic phase was washed with 1 M HCl. The combined aqueous phases were basified to pH 10 with sat. Na₂CO₃ (aq) and extracted with DCM. The combined organic phases were dried through a phase separator and evaporated in vacuo to yield 443 mg (97%) which was used in the next step without further purification.

¹H NMR (CDCl₃) δ 7.12 (d, 1H), 6.87 (dd, 1H), 6.82 (ds, 1H), 3.82 (s, 3H), 3.75 (s, 2H), 1.48 (s, 2H).

### e) tert-butyl 4-{[(4-chloro-2-methoxybenzyl)amino]carbonyl}piperidine-1-carboxylate

1-(tert-butoxycarbonyl)piperidine-4-carboxylic acid (220 mg, 0.961 mmol), HOAt (130 mg, 0.961 mmol) and EDC (181 mg, 0.961 mmol) were dissolved in DCM (8 ml) and stirred for 10 min before addition of 1-(4-chloro-2-methoxyphenyl)methanamine (150 mg, 0.874 mmol) dissolved in DCM (2 ml). The reaction was stirred at rt for 16 h and was then separated between DCM and 0.1 M KHSO₄ (aq). The aqueous phase was extracted with DCM and the combined organic phases were washed twice with 5% Na₂CO₃ (aq), dried through a phase separator and evaporated in vacuo. Purification was done with Biotage Horizon Pioneer® HPFC using a silica cartridge with a gradient elution of 5-50% EtOAc/MeOH/TEA 100:3:0.3 in EtOAc yielding the title compound as a white solid 279 mg (83%).

¹H NMR (CDCl₃) δ 7.08 (d, 1H), 6.82 (dd, 1H), 6.78 (ds, 1H), 6.12 (t, 1H), 4.30 (d, 2H), 4.05 (m, 2H), 3.78 (s, 3H), 2.65 (t, 2H), 2.17 (dt, 1H), 1.71 (dd, 2H), 1.55 (dq, 2H), 1.39 (s, 9H).

### f) N-(4-chloro-2-methoxybenzyl)-1-({1-[4-(trifluoromethyl)phenyl]-1H-pyrrol-3-yl}methyl)piperidine-4-carboxamide

tert-butyl 4-{[(4-chloro-2-methoxybenzyl)amino]carbonyl}piperidine-1-carboxylate (279 mg, 0.727 mmol) was stirred with 4 M HCl in dioxane overnight. The solvent was coevaporated (3×) in vacuo with MeOH. The remaining salt was dissolved in DCM and DIPEA (254 μl, 1.457 mmol). 1-[4-(trifluoromethyl)phenyl]-1H-pyrrole-3-carbaldehyde (192 mg, 0.802 mmol) and NaBH(OAc)₃ (463 mg, 2.186 mmol) were added and the mixture was stirred at rt for 4 h. DCM and 5% Na₂CO₃ (aq) were then added and the phases were separated. The aqueous phase was extracted twice with DCM, the combined organic phases dried through a phase separator and evaporated in vacuo. Purification was done with Biotage Horizon Pioneer® HPFC using a silica cartridge with a gradient elution of 30-100% EtOAc/MeOH/TEA 100:10:1 in EtOAc yielding the title compound as a white solid 283 mg (76%).

¹H NMR (CDCl₃) δ 7.62 (d, 2H), 7.42 (d, 2H), 7.12 (d, 1H), 7.03 (t, 1H), 7.00 (t, 1H), 6.84 (dd, 1H), 6.80 (ds, 1H), 6.28 (dd, 1H), 5.97 (t, 1H), 4.34 (d, 2H), 3.80 (s, 3H), 3.40 (s, 2H), δ 2.98 (dt, 2H), δ 2.06 (m, 1H), δ 1.94 (dt, 2H), 1.84-1.65 (m, 4H).

¹³C NMR (CDCl₃) δ 174.9, 158.2, 143.2, 134.2, 130.6, δ 127.3 (q, J=33.2 Hz), δ 127.1 (q, J=3.7 Hz), δ 125.3, δ 124.3 (q, J=271.6 Hz), δ 123.7, 120.8, 119.6, 119.1, 118.3, 113.2, 111.3, 55.8, 55.6, 53.2, 43.7, 38.8, 29.2.

MS (ESI+) 506.2 (M+1H⁺)

### Example 71

### N-(4-chloro-2-hydroxybenzyl)-1-({1-[4-(trifluoromethyl)phenyl]-1H-pyrrol-3-yl}methyl)piperidine-4-carboxamide, acetate salt

N-(4-chloro-2-methoxybenzyl)-1-({1-[4-(trifluoromethyl)phenyl]-1H-pyrrol-3-yl}methyl)piperidine-4-carboxamide (138 mg, 0.273 mmol, from Fx-amrtple 70) sas dissolved in DCM (10 mL) and cooled in an ice bath under a N₂ atmosphere. Boron tribromide (1 M in DCM, 2.00 mmol) was slowly added and the reaction was then stirred for 2 h. The solvent was removed and 5% NaHCO₃ (aq) (2 ml) was added. The solvent was evaporated in vacuo. Purification was done by preparative HPLC and yielded the title compound as a white solid 41 mg (27%).

¹H NMR (CDCl₃) δ 7.82 (b, 1H), δ 7.64 (d, 2H), δ 7.42 (d, 2H), δ 7.08 (s, 1H), δ 7.05 (t, 1H), 87.02-6.94 (m, 2H), δ 6.83 (d, 1H), δ 6.71 (dd, 1H), δ 6.31 (m, 1H), δ 4.25 (d, 2H), δ 3.61 (s, 2H), δ 3.11 (dt, 2H), δ 2.31-2.17 (m, 3H), δ 2.00 (s, 3H), δ 1.94-1.80 (m, 4H).

MS (ESI+) 492.2 (M+1H⁺)

### Example 72

### 2-(3-fluorophenyl)-N-[1-({1-[4-(trifluoromethyl)phenyl]-1H-pyrrol-3-yl}methyl)piperidin-4-yl]pyrrolidine-1-carboxamide

### a) 4-nitrophenyl[1-({1-[4-(trifluoromethyl)phenyl]-1H-pyrrol-3-yl}methyl)piperidin-4-yl]carbamate

5% aqueous Na₂CO₃ (130 mL) was added to a suspension of 1-({1-[4-(trifluoromethyl)phenyl]-1H-pyrrol-3-yl}methyl)piperidin-4-amine dihydrochloride (2.62 g, 6.61 mmol, from Example C) in DCM (130 ml). The mixture was stirred for 15 min and then the organic phase was separated through a phase separator. Bis(p-nitrophenyl)carbonate (2.01 g, 6.61 mmol) was added and the resulting mixture stirred for 1 h at room temperature. 5% aqueous Na₂CO₃ (130 mL) was added and the mixture stirred for 15 min and then the organic phase was separated through a phase separator, diluted with DCM to a 154 mL stock solution.

### b) 2-(3-fluorophenyl)-N-[1-({1-[4-(trifluoromethyl)phenyl]-1H-pyrrol-3-yl}methyl)piperidin-4-yl]pyrrolidine-1-carboxamide

1 mL of a 0.3M stock solution of DIPEA in DCM and 3.5 mL of a 0.043M stock solution of 4-nitrophenyl[1-({1-[4-(trifluoromethyl)phenyl]-1H-pyrrol-3-yl]}methyl)piperidin-4-yl]carbamate were added to 2-(3-fluorophenyl)pyrrolidine (50 mg, 0.30 mmol). The to resulting mixture was stirred over night. 5% aqueous Na₂CO₃ (5 mL) was added and after 15 min the organic phase was separated through a phase separator and evaporated in a vacuum centrifuge. The remaining oil was purified by Automated Preparative HPLC to give the title compound 0.048 g (62%).

¹H NMR ((CD₃)₂SO) δ 7.74 (s, 1H), 7.40 (t, 1H), 7.33-7.26 (m, 2H), 6.99-6.93 (m, 2H), 6.88 (d, 1H), 6.20-6.17 (m, 1H), 5.64 (d, 1H), 4.88 (dd, 1H), 3.54-3.47 (m, 1H), 3.41-3.25 (m, 4H obscured by H₂O-peak), 2.76-2.65 (m, 2H), 2.22-2.12 (m, 1H), 1.90-1.20 (m, 9H).

MS (ESI+) 515.2 (M+1H⁺).

### Example 73-79

Using the method described for the preparation of the compound of Example 72, the compounds of Example 73-79 were prepared by reaction of 4-nitrophenyl[1-({1-[4-(trifluoromethyl)phenyl]-1H-pyrrol-3-yl}methyl)piperidin-4-yl]carbamate with different amines. The isolated yields of the products were in the range 34-91% with purity in excess of 92% (assessed by HPLC-UV and ¹H NMR)

| | | MS |
|---|---|---|
| | | (ESI+) |
| Example | Compound Name | (M + 1H⁺) |
| 73 | N-[2-(1H-imidazol-1-yl)-1-phenylethyl]-N′-[1-({1-[4- | 537.3 |
| | (trifluoromethyl)phenyl]-1H-pyrrol-3-yl}methyl)piperidin-4-yl]urea | |
| 74 | N-(3-fluorobenzyl)-N-methyl-N′-[1-({1-[4-(trifluoromethyl)phenyl]- | 489.2 |
| | 1H-pyrrol-3-yl}methyl)piperidin-4-yl]urea | |
| 75 | 3-(1,1-dioxidothiomorpholin-4-yl)-N-[1-({1-[4- | 540.2 |
| | (trifluoromethyl)phenyl]-1H-pyrrol-3-yl}methyl)piperidin-4- | |
| 76 | N-(3-hydroxybutyl)-N′-[1-({1-[4-(trifluoromethyl)phenyl]-1H-pyrrol-3- | 439.2 |
| | yl}methyl)piperidin-4-yl]urea | |
| 77 | N-[(1S)-2-hydroxy-1-phenylethyl]-N′-[1-({1-[4- | 487.2 |
| | (trifluoromethyl)phenyl]-1H-pyrrol-3-yl}methyl)piperidin-4-yl]urea | |
| 78 | 2-(1,3-benzothiazol-2-yl)-N-[1-({1-[4-(trifluoromethyl)phenyl]-1H- | 554.2 |
| | pyrrol-3-yl}methyl)piperidin-4-yl]pyrrolidine-1-carboxamide | |
| 79 | 2-(pyridin-3-ylmethyl)-N-[1-({1-[4-(trifluoromethyl)phenyl]-1H- | 512.3 |
| | pyrrol-3-yl}methyl)piperidin-4-yl]pyrrolidine-1-carboxamide | |

### Example 80

### (+)-2-(3-fluorophenyl)-N-[1-({1-[4-(trifluoromethyl)phenyl]-1H-pyrrol-3-yl}methyl)piperidin-4-yl]pyrrolidine-1-carboxamide

2-(3-Fluorophenyl)-N-[1-({1-[4-(trifluoromethyl)phenyl]-1H-pyrrol-3-yl}methyl)piperidin-4-yl]pyrrolidine-1-carboxamide (305 mg, 0.593 nm-iol), the title compound of Example 72, was chromatographed on a Chiralpak AD 250×20 mm column, particle size 10 μm, mobile phase MeOHWTFA 99.9/0.1, flow 15 mL/min, detection 254 nm at room temperature. The injected amount was 19 mg per run. The first peak was collected, evaporated and freeze dried from dioxane to give 126 mg (82% of the theoretical yield), ee 99%.

MS (ESI) 515 (M+1H⁺).

### Example 81

### (−)-2-(3-fluorophenyl)-N-[1-({1-[4-(trifluoromethyl)phenyl]-1H-pyrrol-3-yl}methyl)piperidin-4-yl]pyrrolidine-1-carboxamide

See Example 80. The second peak was collected, evaporated and freeze dried from dioxane to give 136 mg (89%), ee 99%.

MS (ESI) 515 (M+1H⁺).

### Example 82

### (+)-N-[2-(1H-imidazol-1-yl)-1-phenylethyl]-N′-[1-({1-[4-(trifluoromethyl)phenyl]-1H-pyrrol-3-yl}methyl)piperidin-4-yl]urea

N-[2-(1H-imidazol-1-yl)-1-phenylethyl]-N′-[1-({1-[4-(trifluoromethyl)phenyl]-1H-pyrrol-3-yl}methyl)piperidin-4-yl]urea (0.327 mg, 0.609 mmol), the title compound of Example 73, was chromatographed as described in Example 80. The injected amount was 35 mg per run. The first peak was collected, evaporated and freeze dried from dioxane to give 153 mg (94%), ee>99%.

MS (ESI) 537 (M+1H).

### Example 83

### (−)-N-[2-(1H-imidazol-1-yl)-1-phenylethyl]-N′-[1-({1-[4-(trifluoromethyl)phenyl]-1H-pyrrol-3-yl}methyl)piperidin-4-yl]urea

See Example 82. The second peak was collected, evaporated and freeze dried from dioxane to give 158 mg, ee>99%. The chemical purity was not satisfactory and the material was further purified on prep HPLC (Chromasil C8 50×300 mm) using CH₃CN/0.1 M NH₄OAc 10/90->100/0. The acetonitrile was evaporated and the aqueous phase was made alkaline with 2M NaOH and extracted with EtOAc three times. The combined organic layer was washed with water, dried over Na₂SO₄ and evaporated. Yield: 114 mg (70%) of pure product.

MS (ESI) 537 (M+1H⁺).

### Example 84-87

Using the method described for the preparation of the compound of Example 72, the compounds of Example 84-87 were prepared by reaction of 4-nitrophenyl[1-({1-[4-(trifluoromethyl)phenyl]-1H-pyrrol-3-yl}methyl)piperidin-4-yl]carbamate with different amines. The isolated yields of the products were in the range 23-34% with purity in excess of 97% (assessed by HPLC-UV and ¹H NMR).

| | | MS |
|---|---|---|
| | | (ESI+) |
| Example | Compound Name | (M + 1H⁺) |
| 84 | 2-(2-hydroxyethyl)-N-[1-({1-[4-(trifluoromethyl)phenyl]-1H-pyrrol-3- | 479.2 |
| | yl}methyl)piperidin-4-yl]piperidine-1-carboxamide | |
| 85 | N-(4-fluorobenzyl)-N-(3-hydroxypropyl)-N′-[1-({1-[4- | 533.2 |
| | (trifluoromethyl)phenyl]-1H-pyrrol-3-yl}methyl)piperidin-4-yl]urea | |
| 86 | N-(2-hydroxy-3-phenoxypropyl)-N′-[1-({1-[4- | 517.2 |
| | (trifluoromethyl)phenyl]-1H-pyrrol-3-yl}methyl)piperidin-4-yl]urea | |
| 87 | N-[(1-hydroxycyclohexyl)methyl]-N′-[1-({1-[4- | 479.2 |
| | (trifluoromethyl)phenyl]-1H-pyrrol-3-yl}methyl)piperidin-4-yl]urea | |

### Example 88

### N-[(4-fluorophenyl)(6-methoxypyridin-3-yl)methyl]-2-[1-({1-[4-(trifluoromethyl)phenyl]-1H-pyrrol-3-yl}methyl)piperidin-4-yl]acetamide

### a) N,6-dimethoxy-N-methylnicotinamide

Methyl 6-methoxynicotinate (1.500 g, 8.97 mmol) and N,O-dimethylhydroxylamine hydrochloride (2.68 g, 26.02 mmol) were stirred in THF (20 mL) and cooled to −40° C. under argon. Isopropyl magnesium chloride (13 mL, 2M THF solution) was added during 15 minutes and the reaction mixture was stirred for 20 minutes. The reaction was quenched with 20% aq. AcOH, and the reaction mixture was extracted with diethyl ether. The water phase was basified with sat. aq. NaHCO₃ and extracted with DCM three times. The combined organic phases were dried over MgSO₄, filtered and concentrated in vacuo and purified by Biotage Horizon Pioneer® BPFC using a silica cartridge with gradient elution from 5 to 30% EtOAc in n-heptane to give the title compound (1.636 g, 93%).

MS (ESI+) 197.1 (M+1H).

¹H NMR (CDCl₃) δ 8.64 (d, 1H, J=2.3 Hz), 7.98 (dd, 1H, J=2.5 Hz, J=8.8 Hz), 6.77 (d, 1H, J=8.8 Hz), 3.97 (s, 3H), 3.57 (s, 3H), 3.36 (s, 3H).

### b) (4-fluorophenyl)(6-methoxypyridin-3-yl)methanone

N,6-dimethoxy-N-methylnicotinamide (0.500 g, 2.55 mmol) and 1-bromo-4-fluorobenzene (0.445 g, 2.55 mmol) were stirred in dry THF (15 mL) and cooled to −78° C. under argon. n-BuLi (0.326 g, 5.09 mmol, 1.6M THF solution) was added drop wise to the reaction mixture and after 20 minutes of stirring was 1 M aq. HCl (10 mL) added followed by addition of EtOAc (40 mL). The organic phase was washed with water, brine and then dried over MgSO₄, filtered and concentrated in vacuo and purified with by Biotage Horizon Pioneer® HPFC using a silica cartridge with gradient elution from 0 to 15% EtOAc in n-heptane to give the title compound as an clear oil (0.225 g, 38%).

¹H NMR (CDCl₃) δ 8.56 (d, 1H, J=2.3 Hz), 8.04 (dd, 1H, J=2.7 Hz, J=8.9 Hz), 7.84 (m, 2H), 7.15 (m, 2H) 6.82 (d, 1H, J=8.9 Hz), 4.00 (s, 3H).

### c) (E)-(4-fluorophenyl)(6-methoxypyridin-3-yl)methanone oxime

(4-fluorophenyl)(6-methoxypyridin-3-yl)methanone (0.225 g, 0.973 mmol), hydroxylamine hydrochloride (0.270 g, 3.89 mmol) and DIPEA (0.68 mL, 3.89 mmol) was dissolved in EtOH (99.5%, 5 mL) and heated in a microwave oven at 120° C. for 2×30 minutes. Additional hydroxylamine hydrochloride (0.250 g) was added and the reaction mixture was heated at 120° C. for 30 minutes. The reaction mixture was concentrated in vacuo and sat. NaHCO₃ (aq.)/H₂O (9:1, 20 mL) was added and the mixture was extracted with DCM. The combined organic phases were dried over MgSO₄, filtered and concentrated in vacuo to give the title compound (0.240 g, 100%).

MS (ESI+) 247.1 (M+1H⁺), MS (ESI−) 244.9 (M−1H⁺).

### d) [(4-fluorophenyl)(6-methoxypyridin-3-yl)methyl]amine

A mixture of (E)-(4-fluorophenyl)(6-methoxypyridin-3-yl)methanone oxime (0.238 g, 0.97 mmol) and ammonium acetate (0.127 g, 1.64 mmol) in absolute ethanol (6 mL), water (4 mL) and NH₃ (26% aq, 5 mL) was heated to 80° C. Zn powder (0.284 g, 4.35 mmol) was added portionwise to the reaction mixture over 1 hour and then stirred for 4 hour at 80° C. Added sat. NaHCO₃ (aq.)/H₂O (1:1, 20 mL) to the reaction mixture and extracted with DCM three times. The combined organic phases was dried over a phase separator and concentrated in vacuo to give the title compound as a yellow oil (0.197 g, 88%).

MS (ESI+) 234.1 (M+1H⁺).

### e) tert-butyl 4-(2-{[(4-fluorophenyl)(6-methoxypyridin-3-yl)methyl]amino}-2-oxoethyl)piperidine-1-carboxylate

The title compound was synthesised in 0.6 mmol scale using the same procedure as in Example 68, step e, by the use of HOBt instead of HOAt giving the title compound (0.238 g, 86%).

¹H NMR (CDCl₃) δ 8.02 (d, 1H, J=2.5 Hz), 7.36 (dd, 1H, J=2.6 Hz, J=8.9 Hz), 7.16-7.20 (m, 2H), 7.00-7.06 (m, 2H), 6.71 (d, 1H, J=8.3 Hz), 6.20 (d, 1H, J=8.3 Hz), 5.99 (d, 1H, J=7.9 Hz), 4.06 (bs, 2H), 3.92 (s, 3H), 2.96 (m, 2H), 2.16 (d, 2H, J=7.1 Hz), 2.00 (m, 1H), 1.60-1.74 (m, 2H), 1.45 (s, 9H), 1.06-1.18 (m, 2H).

### f) N-[(4-fluorophenyl)(6-methoxypyridin-3-yl)methyl]-2-[1-({1-[4-(trifluoromethyl)phenyl]-1H-pyrrol-3-yl}methyl)piperidin-4-yl]acetamide

The title compound was synthesised in 0.520-mmol scale using the same procedure as in Example 68, step f, but purified with Biotage Horizon Pioneer® HPFC using a silica cartridge and elution by EtOAc:MeOH:TEA (100:5:0.5) to give the title compound as a clear oil (0.220 g, 73%).

¹H NMR (CDCl₃) δ 7.98 (d, 1H, J=2.6 Hz), 7.63 (d, 2H, J=8.5 Hz), 7.43 (d, 2H, J=8.5 Hz), 7.33 (dd, 1H, J=2.7 Hz, J=9.0 Hz), 7.11-7.18 (m, 2H), 6.94-7.06 (m, 3H), 6.65 (d, 1H, J=8.5 Hz), 6.53 (d, 1H, J=8.5 Hz), 6.30 (m, 1H), 6.15 (d, 1H, J=8.1 Hz), 3.87 (s, 3H), 3.40 (s, 2H), 2.92 (m, 2H), 2.76 (s, 1H), 2.11 (d, 2H, J=7.4 Hz), 1.81-1.98 (m, 2H), 1.74-1.84 (m, 1H), 1.60-1.68 (m, 2H), 1.21-1.34 (m, 2H).

MS (ESI+) 581.2 (M+1H⁺), MS (ESI−) 578.9 (M−1H⁺).

### Example 89

### N-[(4-fluorophenyl)(6-oxo-1,6-dihydropyridin-3-yl)methyl]-2-[1-({1-[4-(trifluoromethyl)phenyl]-1H-pyrrol-3-yl}methyl)piperidin-4-yl]acetamide

The title compound was synthesised in 0.344-mmol scale using the same procedure as in Example 69 giving the title compound as an 50% AcOH salt (0.153 g, 78%).

¹H NMR (CD₃OD) δ 7.76 (d, 2H, J=8.3 Hz), 7.69 (d, 2H, J=8.3 Hz), 7.43-7.48 (m, 2H), 7.35 (m, 1H), 7.27-7.32 (m, 2H), 7.19 (m, 1H), 7.07-7.12 (m, 2H), 6.52 (d, 1H, J=9.8 Hz), 6.44 (m, 1H), 6.01 (s, 1H), 3.95 (s, 2H), 3.29-3.37 (m, 2H), 2.64 (m, 2H), 2.27 (d, 2H, J=7.2 Hz), 1.97 (m, 1H), 1.92 (s, 1.5H, AcOH), 1.79-1.88 (m, 2H), 1.43-1.55 (m, 2H).

MS (ESI+) 567.2 (M+1H), MS (ESI−) 578.9 (M −1H⁺).

### Example 90

### N-[2-(2-methyl-1H-imidazol-1-yl)-1-phenylethyl]-N′-[1-({1-[4-(trifluoromethyl)phenyl]-1H-pyrrol-3-yl}methyl)piperidin-4-yl]urea

### a) 2-(2-methyl-1H-imidazol-1-yl)-1-phenylethanone

2-methyl-1H-imidazole (1.07 g, 13.06 mmol) and K₂CO₃ (s) anhydrous (2.78 g, 20.09 mmol) was stirred in acetone (10 ml) for 5 min before addition of 2-bromo-1-phenylethanone (2.00 g, 10.05 mmol). The mixture was stirred at rt for 5 min during which time a milky solution and gas evolution were formed. The mixture was then heated at 140° C. for 15 min in a microwave. The solvent was evaporated. Separated between EtOAc (250 ml) and 5% Na₂CO₃ (aq) (250 ml), the aqueous phase was washed with EtOAc (4×250 ml), the combined organics dried through a phase separator and evaporated in vacuo. Purification was done with Biotage Horizon Pioneer® HPFS using a silica cartridge with a gradient elution of 20-100% EtOAc/MeOH/TEA 100:3:0.3 in EtOAc yielding the title compound as a white solid 1.11 g (48%).

¹H NMR (CDCl₃) δ 7.92 (d, 2H), δ 7.61 (d, 1H), δ 7.48 (d, 2H), δ 6.91 (d, 1H), δ 6.75 (d, 1H), δ 5.25 (s, 2H), δ 2.22 (s, 3H).

### b) 2-(2-methyl-1H-imidazol-1-yl)-1-phenylethanamine

2-(2-methyl-1H-imidazol-1-yl)-1-phenylethanone (400.0 mg, 2.00 mmol), NH₄OAc (3.08 g, 39.95 mmol) and Pol-BH₃CN (1.46 g, 5.99 mmol, 4.1 mmol/g) were dissolved in dry MeOH (10 ml). The reaction was heated in a microwave oven at 150° C. for 15 min. The resin was filtered off and washed with MeOH. The filtrate was collected and evaporated in vacuo and was then partitioned between DCM (150 ml) and 5% Na₂CO₃ (aq) (150 ml). The aqueous phase was extracted with DCM (2×150 ml), the combined organic phases dried through a phase separator and evaporated in vacuo yielding the title compound as a colourless oil, 316 mg (79%).

¹H NMR (CDCl₃) δ 7.34-7.20 (m, 5H), 6.85 (d, 1H), 6.78 (d, 1H), 4.23 (s, 1H), 3.92 (d, 2H), 2.13 (s, 3H), 2.20-1.70 (b, 2H).

### c) N-[2-(2-methyl-1H-imidazol-1-yl)-1-phenylethyl]-N′-[1-({1-[4-(trifluoromethyl)phenyl]-1H-pyrrol-3-yl}methyl)piperidin-4-yl]urea

The title compound was prepared by reaction of 4-nitrophenyl[1-({1-[4-(trifluoromethyl)phenyl]-1H-pyrrol-3-yl}methyl)piperidin-4-yl]carbamate with 2-(2-methyl-1H-imidazol-1-yl)-1-phenylethanamine according to the method described for the preparation of Example 72. Purification was done by preparative HPLC. Separated between DCM (50 ml) and 5% Na₂CO₃ (aq) (50 ml) and extracted the aqueous phase with DCM (2×50 ml). The combined organics were dried through a phase separator and evaporated in vacuo yielding the title compound as a white solid 80 mg (46%).

¹H NMR (CDCl₃) δ 7.60 (d, 2H), 7.38 (d, 2H), 7.21-7.16 (iyi, 3H), 7.02-6.95 (m, 4H), 6.63 (s, 1H), 6.38 (s, 1H), 6.26-6.20 (s, 2H), 5.95 (s, 1H), 5.03 (q, 1H), 4.12 (dd, 1H), 4.04 (dd, 1H), 3.68-3.48 (m, 1H), 3.36 (s, 2H), 2.82 (t, 2H), 2.01 (q, 2H), 1.94 (s, 3H), 1.81 (dd, 2H), 1.32 (m, 2H).

¹³C NMR (CDCl₃) δ 157.8, 145.4, 143.1, 139.5, 129.0, 128.2, 127.4 (q, J=32.8 Hz), 127.1 (q, J=3.6 Hz), 126.8, 126.4, 124.2 (q, J=271.1 Hz), 123.3, 120.3, 119.7, 119.2, 118.5, 113.2, 55.3, 55.0, 52.4, 51.7, 47:2, 33.0, 12.7.

MS (ESI+) 551.3 (M+10H), MS (ESI−) 549.0 (M−1H⁺).

### Pharmacological Properties

### MCH1 Receptor Radioligand Binding.

Assays were performed on membranes prepared from CHO-K1 cells expressing the human Melanin concentrating hormone receptor 1 (hMCHr1, 5.45 pmol/mg protein; Euroscreen). Assays were performed in a 96-well plate format in a final reaction volume of 200 μl per well. Each well contained 6 μg of membrane proteins diluted in binding buffer (50 mM Tris, 3 mM MgCl₂, 0.05% bovine serum albumin and the radioligand ¹²⁵I-MCH (IM344 Amersham) was added to give 10 000 cpm (counts per minute) per well. Each well contained 2 μl of the appropriate concentration of competitive antagonist prepared in DMSO and left to stand at 30° C. for 60 minutes. Non-specific binding was determined as that remaining following incubation with 1 M MCH (Melanin concentrating hormone, H-1482 Bachem). The reaction was terminated by transfer of the reaction to GF/A filters using a Micro96 Harvester (Skatron Instruments, Norway). Filters were washed with assay buffer. Radioligand retained on the filters was quantified using a 1450 Microbeta TRILUX (Wallac, Finland).

Non-specific binding was subtracted from all values determined. Maximum binding was that determined in the absence of any competitor following subtraction of the value determined for non-specific binding. Binding of compounds at various concentrations was plotted according to the equation

y=A+((B−A)1+((C/x)̂D)))

and IC₅₀ estimated where

A is the bottom plateau of the curve i.e. the final minimum y value

B is the top of the plateau of the curve i.e. the final maximum y value

C is the x value at the middle of the curve. This represents the log EC₅₀ value when A+B=100

D is, the slope factor. x is the original known x values. y is the original known y values.

The compounds exemplified herein had an IC₅₀ of less than 1 μM in the above-mentioned human MCHr1 binding assay. Preferred compounds had an activity of less than 0.6 μM; For instance, the following IC₅₀ values were obtained for the compounds of the following examples:

Example 2, 0.012 μM

Example 3, 0.014 μM

Example 6, 0.072 μM

### Mchr1 Functional Assay

Membranes expressing recombinant hMCHr1 (5.45 μmol/mg protein; Euroscreen) were prepared in assay buffer (50 mM HEPES, 100 mM NaCl, 5 mM MgCl₂, 1 mM EDTA, 200 μM DTT, 20 μM GDP (Sigma) containing 0.1 μg/ml BSA, pH7.4) before assay. The assays were performed using membranes at 6 μg/well in an assay volume of 200 μL and the appropriate concentrations of compounds prepared in DMSO. The reaction was started by addition of 0.056 nM [³⁵S]GTPγS (Specific activity >1000 Ci/mmol; Amersham) and an ED₈₀ concentration of MCH (determined for each membrane and each MCH batch). Non-specific binding was determined using 20 μM non-radiolabelled GTPγS. Plates were incubated for 45 min at 30° C. Free and bound GTPγS were separated by filtration binding using GF/B filter mats presoaked in wash buffer (50 mM Tris, 5 mM MgCl₂, 50 mM NaCl, pH 7.4) using a Micro96 cell harvester (Skatron Instruments) and the filters then dried at 50° C. before counting using a 1450 Microbeta TRILUX (Wallac).

Data are means ±SD for experiments performed in triplicate. IC₅₀ values of antagonists were determined using non-linear regression analysis of concentration response curves using Activity Base. For instance, the following IC₅₀ values were obtained for the compounds of the following examples:

Example 1, 0.042 μM

Example 2, 0.112 μM

### Diet Induced Obesity Model in Mouse

The utility of the compounds of the present invention in the treatment of obesity and related conditions is demonstrated by a decrease in body weight in cafeteria diet-induced obese mice. Female C57B1/6J mice were given ad libitum access to calorie-dense ‘cafeteria’ diet (soft chocolate/cocoa-type pastry, chocolate, fatty cheese and nougat) and standard lab chow for 8-10 weeks until a body weight of 45-50 grams was achieved. Compounds to be tested were then administered systemically (iv, ip, sc or po) once daily for a minimum of 5 days, and the body weights of the mice monitored on a daily basis. During this period ad libitum access to calorie-dense ‘cafeteria’ diet and standard lab chow was maintained. Simultaneous assessment of adiposity was carried by means of DEXA imaging at baseline and termination of the study. Blood sampling was also carried out to assay changes in obesity-related plasma markers. Compounds of the invention gave a significant decrease in body weight, with the major effect being via a reduction in fat-mass.

### HERG Activity

HERG testing was performed using a modified version of the method described by Kiss L, Bennett P B, Uebele V N, Koblan K S, Kane S A, Neagle B, Schroeder K. “High throughput ion-channel pharmacology: planar-array-based voltage clamp” Assay Drug Dev Technol. 1, 127-35. (2003). For example, the compounds of Examples 76 and 83 had IC₅₀ values exceeding 5 μM in the abovementioned assay.

Compounds of the invention have the advantage that they may be more potent, more selective (e.g. vs. ion channels such as HERG and/or vs. GPCR's related to MCHr1) more efficacious in vivo, be less toxic, be longer acting, produce fewer side effects, be more easily absorbed, be less metabolised and/or have a better pharmacokinetic profile than, or have other useful pharmacological or physicochemical properties over, compounds known in the prior art.

1. A compound of formula I 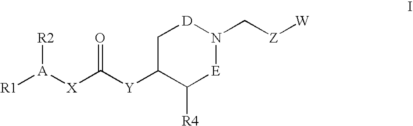
A represents N, a C₁₋₄ alkyl group, a C₂₋₄ alkenyl group, C₃₋₈ cycloalkyl, adamantyl, azetidinyl, pyrrolidinyl, piperidinyl, piperazinyl, morpholinyl, 1,3 oxazidinyl, tetrahydropyridinyl, or spiro[indene-1,4′-piperidinyl]; wherein said C₁₋₄ alkyl group or C₂₋₄ alkenyl group is optionally substituted by one or more fluoro;
X represents a bond or NR³,
wherein A and X do not both represent nitrogen;
wherein when A is azetidinyl, 1,3 oxazidinyl, pyrrolidinyl, piperidinyl, piperazinyl, morpholinyl, tetrahydropyridinyl, or spiro[indene-1,4′-piperidinyl]; the nitrogen atom in A is directly attached to C(O),
R¹ and R² independently represent H, C₁₋₆ alkyl, a C₂₋₆ alkenyl group, C₃₋₁₀ cycloalkyl, CONR^{a}R^{b} in which R^{a} and R^{b} independently represent H, a C₁₋₄ alkyl group or R^{a} and R^{b}, together with the nitrogen to which they are attached, form a 4 to 8 membered heterocyclic ring; phenyl or naphthyl; or a heterocyclic group selected from pyrrolyl, imidazolyl, furyl, thienyl, thiazolyl, isothiazolyl, thiadiazolyl, pyrazolyl, oxazolyl, isoxazolyl, pyridyl, pyrazinyl, pyrimidinyl, pyridazinyl, quinolinyl, isoquinolyl, quinazolyl, indolyl, benzofuranyl, benzo[b]thienyl, benzimidazolyl, benzothiazolyl, 1,4-benzodioxinyl, 1,3-benzodioxolyl, piperidinyl, morpholinyl, 1,4-oxazepanyl, or 4,4-dioxothiomorpholinyl;
wherein R¹ or R² are optionally substituted by one or more of the following:
cyano
halo
hydroxy
oxo a C₁₋₄ alkyl group optionally substituted by one or more fluoro;
a C₁₋₄ alkoxy group optionally substituted by one or more fluoro;
a group NCOR^{a}R^{b} or CONR^{a}R^{b} in which R^{a} and R^{b} independently represent a C₁₋₃ alkyl group;
a group SO₂C₁₋₄alkyl, optionally substituted by one or more fluoro;
an aryl or heteroaryl group selected from thiadiazolyl, pyrazolyl, phenyl, phenoxy, 2-pyridyl or 3-pyridyl wherein said aryl or heteroaryl group may optionally be further substituted by one or more of the following:
cyano,
halo,
hydroxy,
a C₁₋₄ alkyl group optionally substituted by one or more fluoro;
a C₁₋₄ alkoxy group optionally substituted by one or more fluoro;
a group NCOR^{a}R^{b} or CONR^{a}R^{b} in which R^{a} and R^{b} independently represent a C₁₋₃ alkyl group;
a group SO₂C₁₋₄alkyl, optionally substituted by one or more fluoro;
R¹ and/or R² is optionally linked to A via oxygen or via a C₁₋₄ alkyl group, wherein one of the carbon atoms in said C₁₋₄ alkyl group optionally is replaced with an oxygen atom,
Y represents NR³, C(R⁵, R⁶) or a bond, wherein at least one of A, X or Y is N, NR³ or a nitrogen-containing heterocyclic ring,
R³, R⁵ and R⁶ independently represent H or a C₁₋₄ alkyl group,
D represents (CH₂)ₙ, wherein n is 0 or 1 and E represents (CH₂)ₘ, wherein m is 0 or 1,
R⁴ represents H or, when m and n are both 1, R₄ represents H or F,
Z represents 2,5-thienyl, 2,5-furyl, or pyrrolyl, optionally substituted by one or more of the following: cyano, halo, a C₁₋₄ alkyl group optionally substituted by one or more fluoro, a C₁₋₄ alkoxy group optionally substituted by one or more fluoro,
W represents phenyl, 2-pyridyl or 3-pyridyl each of which is optionally substituted by one or more of the following: cyano, halo, a C₁₋₄ alkyl group optionally substituted by one or more fluoro, a C₁₋₄ alkoxy group optionally substituted by one or more fluoro, a trifluoromethylsulfonyl or a 2,2′-difluoro-oxolanyl group (fused with two adjacent aromatic carbon atoms in W),
as well as a tautomer, an optical isomer and a racemate thereof as well as a pharmaceutically acceptable salt thereof,
with the proviso that when Y represents NR³ then A-X does not represent OCH₂, CH₂CH₂ or CH═CH, wherein each of the carbon atom may optionally be substituted by 1 to 2 methyl groups and/or 1 to 2 fluoro.

2. A compound of formula Ia 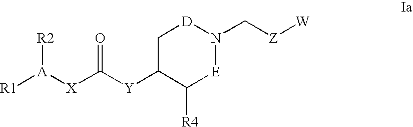
A represents N, a C₁₋₄ alkyl group, a C₂₋₄ alkenyl group, C₃₋₈ cycloalkyl, adamantyl, pyrrolidinyl, piperidinyl, piperazinyl, morpholinyl, tetrahydropyridinyl, or spiro[indene-1,4′-piperidinyl]; wherein said C₁₋₄ alkyl group or C₂₋₄ alkenyl group is optionally substituted by one or more fluoro;
X represents a bond or NR³,
wherein A and X do not both represent nitrogen;
wherein when A is pyrrolidinyl, piperidinyl, piperazinyl, morpholinyl, tetrahydropyridinyl, or spiro[indene-1,4′-piperidinyl]; the nitrogen atom in A is directly attached to C(O),
R¹ and R² independently represent H, C₁₋₆ alkyl, a C₂₋₆ alkenyl group, C₃₋₈ cycloalkyl, CONR^{a}R^{b} in which R^{a} and R^{b} independently represent H, a C₁₋₄ alkyl group or R^{a} and R^{b}, together with the nitrogen to which they are attached, form a 4 to 8 membered heterocyclic ring; phenyl or naphthyl; or a heterocyclic group selected from pyrrolyl, imidazolyl, furyl, thienyl, thiazolyl, isothiazolyl, thiadiazolyl, pyrazolyl, oxazolyl, isoxazolyl, pyridyl, pyrazinyl, pyrimidinyl, pyridazinyl, quinolinyl, isoquinolyl, quinazolyl, indolyl, benzofuranyl, benzo[b]thienyl, benzimidazolyl, benzothiazolyl, 1,4-benzodioxinyl or 1,3-benzodioxolyl;
wherein R¹ or R² are optionally substituted by one or more of the following:
cyano
halo
hydroxy a C₁₋₄ alkyl group optionally substituted by one or more fluoro;
a C₁₋₄ alkoxy group optionally substituted by one or more fluoro;
a group NCOR^{a}R^{b} or CONR^{a}R^{b} in which R^{a} and R^{b} independently represent a C₁₋₃ alkyl group;
a group SO₂C₁₋₄alkyl, optionally substituted by one or more fluoro;
an aryl or heteroaryl group selected from thiadiazolyl, pyrazolyl, phenyl, phenoxy, 2-pyridyl or 3-pyridyl wherein said aryl or heteroaryl group may optionally be further substituted by one or more of the following:
cyano,
halo,
hydroxy,
a C₁₋₄ alkyl group optionally substituted by one or more fluoro;
a C₁₋₄ alkoxy group optionally substituted by one or more fluoro;
a group NCOR^{a}R^{b} or CONR^{a}R^{b} in which R^{a} and R^{b} independently represent a C₁₋₃ alkyl group;
a group SO₂C₁₋₄alkyl, optionally substituted by one or more fluoro;
R¹ and/or R² is optionally linked to A via oxygen or via a C₁₋₄ alkyl group, wherein one of the carbon atoms in said C₁₋₄ alkyl group optionally is replaced with an oxygen atom,
Y represents NR³, C(R⁵, R⁶) or a bond,
wherein at least one of A, X or Y is N, NR³ or a nitrogen-containing heterocyclic ring,
R³, R⁵ and R⁶ independently represent H or a C₁₋₄ alkyl group,
D represents (CH₂)ₙ, wherein n is 0 or 1 and E represents (CH₂)ₘ, wherein m is 0 or 1,
R⁴ represents H or, when m and n are both 1, R₄ represents H or F,
Z represents 2,5-thienyl, 2,5-furyl, or pyrrolyl, optionally substituted by one or more of the following: cyano, halo, a C₁₋₄ alkyl group optionally substituted by one or more fluoro, a C₁₋₄ alkoxy group optionally substituted by one or more fluoro,
W represents phenyl, 2-pyridyl or 3-pyridyl each of which is optionally substituted by one or more of the following: cyano, halo, a C₁₋₄ alkyl group optionally substituted by one or more fluoro, a C₁₋₄ alkoxy group optionally substituted by one or more fluoro, a trifluoromethylsulfonyl or a 2,2′-difluoro-oxolanyl group (fused with two adjacent aromatic carbon atoms in W),
as well as a tautomer, an optical isomer and a racemate thereof as well as a pharmaceutically acceptable salt thereof,
with the proviso that when Y represents NR³ then A-X does not represent OCH₂, CH₂CH₂ or CH═CH, wherein each of the carbon atom may optionally be substituted by 1 to 2 methyl groups and/or 1 to 2 fluoro.

3. A compound according to claim 1, in which all compounds covered by claim 1 in WO 01/14333 are excluded.

4. A compound according to claim 1, in which Y is CH₂.

5. A compound according to claim 1, in which Z is 1,3-1H pyrrolyl (in which the heteroatom is connected to W).

6. A compound according to claim 1, in which W is phenyl or 2- or 3-pyridyl substituted by trifluoromethyl.

7. A compound according to claim 1, in which A is NH, X is a bond and Y is CH₂.

8. A compound according to claim 1, in which A is C₁₋₄ alkyl, X is NH and Y is CH₂.

9. A compound according to claim 1, in which A is NH, X is a bond and Y is a bond.

10. A compound according to claim 1, in which A is C₁₋₄ alkyl, X is NH and Y is a bond.

11. A compound according to claim 1, in which D represents (CH₂)ₙ, wherein n is 1 and E represents (CH₂)ₘ, wherein m is 1.

12. A compound according to claim 1, in which D represents (CH₂)ₙ, wherein n is 1 and E represents (CH₂)ₘ, wherein m is 0, or vice versa.

13. A compound according to claim 1, in which A represents pyrrolidinyl, piperidinyl, piperazinyl, or morpholinyl.

14. A compound according to claim 1, in which A represents piperidinyl.

15. One or more of the following compounds:
2,2-diphenyl-N-[1-({1-[4-(trifluoromethyl)phenyl]-1H-pyrrol-3-yl}methyl)piperidin-4-yl]acetamide,
N-(3,4-difluorobenzyl)-2-[1-({1-[4-(trifluoromethyl)phenyl]-1H-pyrrol-3-yl}methyl)piperidin-4-yl]acetamide,
N-(2-phenylethyl)-1-({1-[4-(trifluoromethyl)phenyl]-1H-pyrrol-3-yl}methyl)piperidine-4-carboxamide,
N-[bis(4-fluorophenyl)methyl]-2-[1-({1-[5-(trifluoromethyl)pyridin-2-yl]-1H-pyrrol-3-yl}methyl)piperidin-4-yl]acetamide,
N,N-bis(4-fluorophenyl)-N′-[1-({1-[5-(trifluoromethyl)pyridin-2-yl]-1H-pyrrol-3-yl}methyl)piperidin-4-yl]urea,
N-[bis(4-fluorophenyl)methyl]-2-[1-({1-[5-(trifluoromethyl)pyridin-2-yl]-1H-pyrrol-3-yl}methyl)pyrrolidin-3-yl]acetamide,
N-(4-fluorophenyl)-1-({1-[5-(trifluoromethyl)pyridin-2-yl]-1H-pyrrol-3-yl}methyl)piperidine-4-carboxamide acetate,
N-(1,3-benzothiazol-2-ylmethyl)-2-[1-({1-[4-(trifluoromethyl)phenyl]-1H-pyrrol-3-yl}methyl)piperidin-4-yl]acetamide,
N-(2-furylmethyl)-2-[1-({1-[4-(trifluoromethyl)phenyl]-1H-pyrrol-3-yl}methyl)piperidin-4-yl]acetamide,
N-(2-pyridin-2-ylethyl)-2-[1-({1-[4-(trifluoromethyl)phenyl]-1H-pyrrol-3-yl}methyl)piperidin-4-yl]acetamide,
N-(2,4-dichlorobenzyl)-2-[1-({1-[4-(trifluoromethyl)phenyl]-1H-pyrrol-3-yl}methyl)piperidin-4-yl]acetamide,
N-(1,2-diphenylethyl)-2-[1-({1-[4-(trifluoromethyl)phenyl]-1H-pyrrol-3-yl}methyl)piperidin-4-yl]acetamide
N-(1,3-benzodioxol-5-ylmethyl)-2-[1-({1-[4-(trifluoromethyl)phenyl]-1H-pyrrol-3-yl}methyl)piperidin-4-yl]acetamide,
N-ethyl-N-(2-pyridin-2-ylethyl)-2-[1-({1-[4-(trifluoromethyl)phenyl]-1H-pyrrol-3-yl}methyl)piperidin-4-yl]acetamide,
N-(2,3-dihydro-1,4-benzodioxin-2-ylmethyl)-2-[1-({1-[4-(trifluoromethyl)phenyl]-1H-pyrrol-3-yl}methyl)piperidin-4-yl]acetamide,
N-[3-(1H-imidazol-1-yl)propyl]-2-[1-({1-[4-(trifluoromethyl)phenyl]-1H-pyrrol-3-yl}methyl)piperidin-4-yl]acetamide,
N-(2,4-dichlorobenzyl)-2-[1-({1-[4-(trifluoromethyl)phenyl]-1H-pyrrol-3-yl}methyl)piperidin-4-yl]acetamide,
N-(4-fluorophenyl)-2-[1-({1-[4-(trifluoromethyl)phenyl]-1H-pyrrol-3-yl}methyl)piperidin-4-yl]acetamide,
N-[phenyl(pyridin-2-yl)methyl]-2-[1-({1-[4-(trifluoromethyl)phenyl]-1H-pyrrol-3-yl}methyl)piperidin-4-yl]acetamide,
N-[3-(difluoromethoxy)benzyl]-2-[1-({1-[4-(trifluoromethyl)phenyl]-1H-pyrrol-3-yl}methyl)piperidin-4-yl]acetamide,
1-(3-methoxyphenyl)-4-{[1-({1-[4-(trifluoromethyl)phenyl]-1H-pyrrol-3-yl}methyl)piperidin-4-yl]acetyl}piperazine,
1′-{[1-({1-[4-(trifluoromethyl)phenyl]-1H-pyrrol-3-yl}methyl)piperidin-4-yl]acetyl}spiro[indene-1,4′-piperidine],
N-(3,3-diphenylpropyl)-2-[1-({1-[4-(trifluoromethyl)phenyl]-1H-pyrrol-3-yl}methyl)piperidin-4-yl]acetamide,
N-(1-phenylpropyl)-2-[1-({1-[4-(trifluoromethyl)phenyl]-1H-pyrrol-3-yl}methyl)piperidin-4-yl]acetamide
N-(4-fluorophenyl)-N-methyl-2-[1-({1-[4-(trifluoromethyl)phenyl]-1H-pyrrol-3-yl}methyl)piperidin-4-yl]acetamide
N-[(1R,2S)-2-phenylcyclopropyl]-2-[1-({1-[4-(trifluoromethyl)phenyl]-1H-pyrrol-3-yl}methyl)piperidin-4-yl]acetamide,
N-(3-methylbutyl)-2-[1-({1-[4-(trifluoromethyl)phenyl]-1H-pyrrol-3-yl}methyl)piperidin-4-yl]acetamide,
N-[2-(3,4-dimethoxyphenyl)ethyl]-N-methyl-2-[1-({1-[4-(trifluoromethyl)phenyl]-1H-pyrrol-3-yl}methyl)piperidin-4-yl]acetamide
N-[2-(3,4-dimethoxyphenyl)ethyl]-2-[1-({1-[4-(trifluoromethyl)phenyl]-1H-pyrrol-3-yl}methyl)piperidin-4-yl]acetamide,
N-[4-(1,2,3-thiadiazol-4-yl)benzyl]-2-[1-({1-[4-(trifluoromethyl)phenyl]-1H-pyrrol-3-yl}methyl)piperidin-4-yl]acetamide,
N-[1-(2,3-dihydro-1,4-benzodioxin-5-yl)ethyl]-2-[1-({1-[4-(trifluoromethyl)phenyl]-1H-pyrrol-3-yl}methyl)piperidin-4-yl]acetamide,
N,N-diethyl-1-{[1-({1-[4-(trifluoromethyl)phenyl]-1H-pyrrol-3-yl}methyl)piperidin-4-yl]acetyl}piperidine-3-carboxamide,
N-1-adamantyl-2-[1-({1-[4-(trifluoromethyl)phenyl]-1H-pyrrol-3-yl}methyl)piperidin-4-yl]acetamide,
N-[2-(4-methoxyphenoxy)ethyl]-2-[1-({1-[4-(trifluoromethyl)phenyl]-1H-pyrrol-3-yl}methyl)piperidin-4-yl]acetamide
N-{(1S)-1-[(benzyloxy)methyl]propyl}-2-[1-({1-[4-(trifluoromethyl)phenyl]-1H-pyrrol-3-yl}methyl)piperidin-4-yl]acetamide
N-[(1R)-1-(3-methoxyphenyl)ethyl]-2-[1-({1-[4-(trifluoromethyl)phenyl]-1H-pyrrol-3-yl}methyl)piperidin-4-yl]acetamide
N-{[3-(4-methoxyphenyl)isoxazol-5-yl]methyl}-2-[1-({1-[4-(trifluoromethyl)phenyl]-1H-pyrrol-3-yl}methyl)piperidin-4-yl]acetamide
4-(4-chlorophenyl)-1-{[1-({1-[4-(trifluoromethyl)phenyl]-1H-pyrrol-3-yl}methyl)piperidin-4-yl]acetyl}-1,2,3,6-tetrahydropyridine
N-[(1S,2S)-2-(benzyloxy)cyclopentyl]-2-[1-({1-[4-(trifluoromethyl)phenyl]-1H-pyrrol-3-yl}methyl)piperidin-4-yl]acetamide
N-(1-methyl-1-phenylethyl)-2-[1-({1-[4-(trifluoromethyl)phenyl]-1H-pyrrol-3-yl}methyl)piperidin-4-yl]acetamide
N-[(1-methyl-1H-pyrrol-2-yl)methyl]-2-[1-({1-[4-(trifluoromethyl)phenyl]-1H-pyrrol-3-yl}methyl)piperidin-4-yl]acetamide
4-(2-oxo-2-pyrrolidin-1-ylethyl)-1-({1-[4-(trifluoromethyl)phenyl]-1H-pyrrol-3-yl}methyl)piperidine,
N-(2-pyridin-2-ylethyl)-1-({1-[4-(trifluoromethyl)phenyl]-1H-pyrrol-3-yl}methyl)piperidine-4-carboxamide,
N-(2,4-dichlorobenzyl)-1-({1-[4-(trifluoromethyl)phenyl]-1H-pyrrol-3-yl}methyl)piperidine-4-carboxamide,
N-(1,2-diphenylethyl)-1-({1-[4-(trifluoromethyl)phenyl]-1H-pyrrol-3-yl}methyl)piperidine-4-carboxamide,
N-(1,3-benzodioxol-5-ylmethyl)-1-({1-[4-(trifluoromethyl)phenyl]-1H-pyrrol-3-yl}methyl)piperidine-4-carboxamide,
N-[2-(3,4-dimethoxyphenyl)ethyl]-1-({1-[4-(trifluoromethyl)phenyl]-1H-pyrrol-3-yl}methyl)piperidine-4-carboxamide,
N-[4-(1,2,3-thiadiazol-4-yl)benzyl]-1-({1-[4-(trifluoromethyl)phenyl]-1H-pyrrol-3-yl}methyl)piperidine-4-carboxamide,
N-(2,3-dihydro-1,4-benzodioxin-2-ylmethyl)-1-({1-[4-(trifluoromethyl)phenyl]-1H-pyrrol-3-yl}methyl)piperidine-4-carboxamide,
N-[1-(2,3-dihydro-1,4-benzodioxin-5-yl)ethyl]-1-({1-[4-(trifluoromethyl)phenyl]-1H-pyrrol-3-yl}methyl)piperidine-4-carboxamide,
N-[phenyl(pyridin-2-yl)methyl]-1-({1-[4-(trifluoromethyl)phenyl]-1H-pyrrol-3-yl}methyl)piperidine-4-carboxamide,
N-[3-(difluoromethoxy)benzyl]-1-({1-[4-(trifluoromethyl)phenyl]-1H-pyrrol-3-yl}methyl)piperidine-4-carboxamide,
N-[2-(4-methoxyphenoxy)ethyl]-1-({1-[4-(trifluoromethyl)phenyl]-1H-pyrrol-3-yl}methyl)piperidine-4-carboxamide,
N-{(1S)-1-[(benzyloxy)methyl]propyl}-({1-[4-(trifluoromethyl)phenyl]-1H-pyrrol-3-yl}methyl)piperidine-4-carboxamide,
N-{[3-(4-methoxyphenyl)isoxazol-5-yl]methyl}-({1-[4-(trifluoromethyl)phenyl]-1H-pyrrol-3-yl}methyl)piperidine-4-carboxamide,
1-(3-methoxyphenyl)-4-{[1-({1-[4-(trifluoromethyl)phenyl]-1H-pyrrol-3-yl}methyl)piperidin-4-yl]carbonyl}piperazine,
4-(4-chlorophenyl)-1-{[1-({1-[4-(trifluoromethyl)phenyl]-1H-pyrrol-3-yl}methyl)piperidin-4-yl]carbonyl}-1,2,3,6-tetrahydropyridine,
N-[(1S,2S)-2-(benzyloxy)cyclopentyl]-1-({1-[4-(trifluoromethyl)phenyl]-1H-pyrrol-3-yl}methyl)piperidine-4-carboxamide,
N-(3,3-diphenylpropyl)-1-({1-[4-(trifluoromethyl)phenyl]-1H-pyrrol-3-yl}methyl)piperidine-4-carboxamide,
N-(1-phenylpropyl)-1-({1-[4-(trifluoromethyl)phenyl]-1H-pyrrol-3-yl}methyl)piperidine-4-carboxamide,
N-(1,3-benzothiazol-2-ylmethyl)-1-({1-[4-(trifluoromethyl)phenyl]-1H-pyrrol-3-yl}methyl)piperidine-4-carboxamide,
N-[(5-chloro-6-methoxypyridin-3-yl)(4-fluorophenyl)methyl]-2-[1-({1-[4-(trifluoromethyl)phenyl]-1H-pyrrol-3-yl}methyl)piperidin-4-yl]acetamide,
N-[(5-chloro-6-oxo-1,6-dihydropyridin-3-yl)(4-fluorophenyl)methyl]-2-[1-({1-[4-(trifluoromethyl)phenyl]-1H-pyrrol-3-yl}methyl)piperidin-4-yl]acetamide acetate salt,
N-(4-chloro-2-methoxybenzyl)-1-({1-[4-(trifluoromethyl)phenyl]-1H-pyrrol-3-yl}methyl)piperidine-4-carboxamide,
N-(4-chloro-2-hydroxybenzyl)-1-({1-[4-(trifluoromethyl)phenyl]-1H-pyrrol-3-yl}methyl)piperidine-4-carboxamide, acetate salt, 2-(3-fluorophenyl)-N-[1-({1-[4-(trifluoromethyl)phenyl]-1H-pyrrol-3-yl}methyl)piperidin-4-yl]pyrrolidine-1-carboxamide,
N-[2-(1H-imidazol-1-yl)-1-phenylethyl]-N′-[1-({1-[4-(trifluoromethyl)phenyl]-1H-pyrrol-3-yl}methyl)piperidin-4-yl]urea,
N-(3-fluorobenzyl)-N-methyl-N′-[1-({1-[4-(trifluoromethyl)phenyl]-1H-pyrrol-3-yl}methyl)piperidin-4-yl]urea,
3-(1,1-dioxidothiomorpholin-4-yl)-N-[1-({1-[4-(trifluoromethyl)phenyl]-1H-pyrrol-3-yl}methyl)piperidin-4-yl]azetidine-1-carboxamide,
N-(3-hydroxybutyl)-N′-[1-({1-[4-(trifluoromethyl)phenyl]-1H-pyrrol-3-yl}methyl)piperidin-4-yl]urea,
N-[(1S)-2-hydroxy-1-phenylethyl]-N′-[1-({1-[4-(trifluoromethyl)phenyl]-1H-pyrrol-3-yl}methyl)piperidin-4-yl]urea,
2-(1,3-benzothiazol-2-yl)-N-[1-({1-[4-(trifluoromethyl)phenyl]-1H-pyrrol-3-yl}methyl)piperidin-4-yl]pyrrolidine-1-carboxamide,
2-(pyridin-3-ylmethyl)-N-[1-({1-[4-(trifluoromethyl)phenyl]-1H-pyrrol-3-yl}methyl)piperidin-4-yl]pyrrolidine-1-carboxamide,
(+)-2-(3-fluorophenyl)-N-[1-({1-[4-(trifluoromethyl)phenyl]-1H-pyrrol-3-yl}methyl)piperidin-4-yl]pyrrolidine-1-carboxamide,
(−)-2-(3-fluorophenyl)-N-[1-({1-[4-(trifluoromethyl)phenyl]-1H-pyrrol-3-yl}methyl)piperidin-4-yl]pyrrolidine-1-carboxamide,
(+)-N-[2-(1H-imidazol-1-yl)-1-phenylethyl]-N′-[1-({1-[4-(trifluoromethyl)phenyl]-1H-pyrrol-3-yl}methyl)piperidin-4-yl]urea,
(−)-N-[2-(1H-imidazol-1-yl)-1-phenylethyl]-N′-[1-({1-[4-(trifluoromethyl)phenyl]-1H-pyrrol-3-yl}methyl)piperidin-4-yl]urea,
2-(2-hydroxyethyl)-N-[1-({1-[4-(trifluoromethyl)phenyl]-1H-pyrrol-3-yl}methyl)piperidin-4-yl]piperidine-1-carboxamide;
N-(4-fluorobenzyl)-N-(3-hydroxypropyl)-N′-[1-({1-[4-(trifluoromethyl)phenyl]-1H-pyrrol-3-yl}methyl)piperidin-4-yl]urea;
N-(2-hydroxy-3-phenoxypropyl)-N′-[1-({1-[4-(trifluoromethyl)phenyl]-1H-pyrrol-3-yl}methyl)piperidin-4-yl]urea;
N-[(1-hydroxycyclohexyl)methyl]-N′-[1-({1-[4-(trifluoromethyl)phenyl]-1H-pyrrol-3-yl}methyl)piperidin-4-yl]urea;
N-[(4-fluorophenyl)(6-methoxypyridin-3-yl)methyl]-2-[1-({1-[4-(trifluoromethyl)phenyl]-1H-pyrrol-3-yl}methyl)piperidin-4-yl]acetamide;
N-[(4-fluorophenyl)(6-oxo-1,6-dihydropyridin-3-yl)methyl]-2-[1-({1-[4-(trifluoromethyl)phenyl]-1H-pyrrol-3-yl}methyl)piperidin-4-yl]acetamide; and
N-[2-(2-methyl-1H-imidazol-1-yl)-1-phenylethyl]-N′-[1-({1-[4-(trifluoromethyl)phenyl]-1H-pyrrol-3-yl}methyl)piperidin-4-yl]urea;

16. N-[4-(trifluoromethoxy)phenyl]-N′-[1-({1-[4-(trifluoromethyl)phenyl]-1H-pyrrol-3-yl}methyl)piperidin-4-yl]urea or a pharmaceutically acceptable salt thereof.

17. N-(2,4-dichlorophenyl)-N′-[1-({1-[4-(trifluoromethyl)phenyl]-1H-pyrrol-3-yl}methyl)piperidin-4-yl]urea pharmaceutically acceptable salt thereof.

18. N-1-naphthyl-N′-[1-({1-[4-(trifluoromethyl)phenyl]-1H-pyrrol-3-yl}methyl)piperidin-4-yl]urea or a pharmaceutically acceptable salt thereof.

19. N-(3-fluorobenzyl)-N′-[1-({1-[4-(trifluoromethyl)phenyl]-1H-pyrrol-3-yl}methyl)piperidin-4-yl]urea, or a pharmaceutically acceptable salt thereof.

20. N-(diphenylmethyl)-N′-[1-({1-[4-(trifluoromethyl)phenyl]-1H-pyrrol-3-yl}methyl)piperidin-4-yl]urea, or a pharmaceutically acceptable salt thereof.

21. N-methyl-N-phenyl-N′-[1-({1-[5-(trifluoromethyl)pyridin-2-yl]-1H-pyrrol-3-yl}methyl)piperidin-4-yl]urea or a pharmaceutically acceptable salt thereof.

22. A compound according to claim 1, in which A is C₁ alkyl, X is NH and Y is NH.

23. (canceled)

24. A pharmaceutical formulation comprising a compound claim 1 and a pharmaceutically acceptable adjuvant, diluent or carrier.

25-26. (canceled)

27. A process for the preparation of a compound of claim 3 comprising reacting a compound of formula II with a compound of formula III 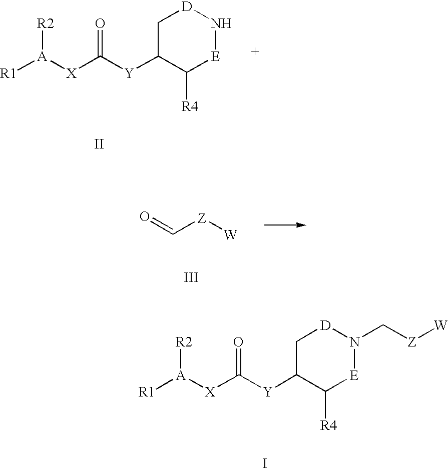
in which A, X, Y, D, E, Z, W, R¹, R² and R⁴ are as previously defined.

28. A process for the preparation of a compound of claim 3 comprising reacting a compound of formula IV with a compound of formula V 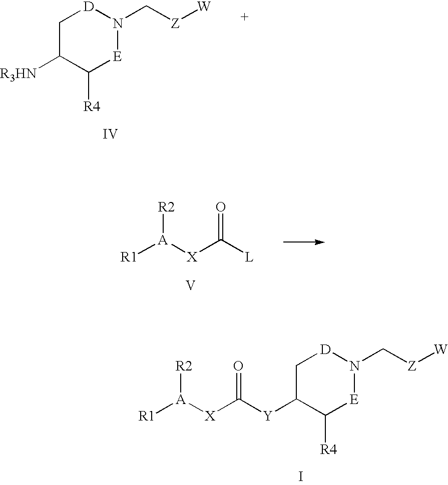
in which A, X, L, Y, D, E, Z, W, R¹, R² and R⁴ are as previously defined.

29. A process for the preparation of a compound of claim 3 comprising reacting a compound of formula VI with a compound of formula IV 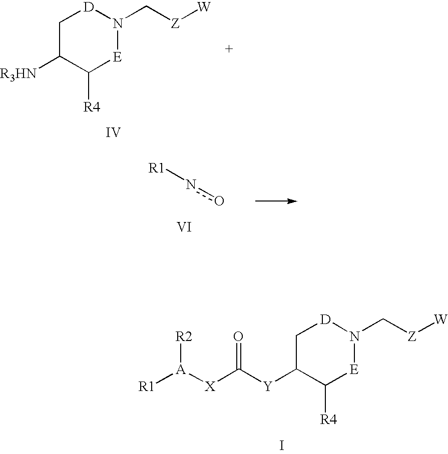
in which A, X, Y, D, E, Z, W, R¹, R² and R⁴ are as previously defined.

30. A process for the preparation of a compound of claim 3 comprising reacting a compound of formula VI with a compound of formula IV 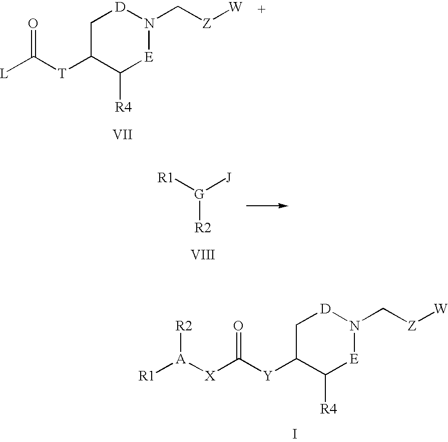
in which A, X, Y, D, E, Z, W, R¹, R², R⁴, L, T, G and J are as previously defined.

31. A method of treating obesity, a psychiatric disorder, anxiety, an anxio-depressive disorder, depression, bipolar disorder, ADHD, a cognitive disorder, memory disorders a memory disorder, schizophrenia, epilepsy, or a related condition, or a neurological disorder or a pain related disorder, comprising administering a pharmacologically effective amount of a compound as claimed in claim 1 to a patient in need thereof.

32. A method of treating obesity, type II diabetes, metabolic syndrome and or prevention of type II diabetes comprising administering a pharmacologically effective amount of a compound as claimed in claim 1 to a patient in need thereof.

33. A pharmaceutical formulation comprising a compound of claim 2 and a pharmaceutically acceptable adjuvant, diluent or carrier.
